(19) United States
(12) Patent Application Publication
(10) Pub. No.: US 2026/0022097 A1
(43) Pub. Date: Jan. 22, 2026

(54) SELECTIVE OPIOID RECEPTOR AGONISTS AND ANTAGONISTS

(71) Applicant: THE UNITED STATES OF AMERICA, AS REPRESENTED BY THE SECRETARY, DEPARTMENT OF HEALTH & HUMAN SERVICES, Bethesda, MD (US)

(72) Inventors: Kenner Cralle RICE, Bethesda, MD (US); Arthur E. JACOBSON, Rockville, MD (US); Agnieszka SULIMA, Poolesville, MD (US); Dana Rae CHAMBERS, Bethlehem, CT (US); Hudson Gordon ROTH, Rockville, MD (US)

(21) Appl. No.: 19/099,189

(22) PCT Filed: Jul. 26, 2023
(86) PCT No.: PCT/US2023/071022

§ 371 Date: Jan. 28, 2025

Related U.S. Application Data
(60) Provisional application No. 63/393,035, filed on Jul. 28, 2022.

(51) Int. Cl. C07D 221/22; C07D 401/04; C07D 401/06

(52) U.S. Cl. CPC C07D 221/22; C07D 401/04; C07D 401/06

(57) ABSTRACT

Compounds having opioid characteristics are provided that can be used for various therapeutic methods including the treatment of pain and opioid use disorders, and compounds having opioid antagonist properties that can be used to treat opioid overdoses. These compounds include agonists and antagonists having selective activity toward more one or more opioid receptors. Such compounds can be full or partial agonists, or can be antagonists lacking agonist properties toward delta and kappa opioid receptors. The partial agonists can diminish side effects associated with traditional opioid medications, and the antagonists can overcome respiratory depression caused by potent narcotics. Structurally these compounds include constrained piperidines with alkenyl or cyanoalkyl groups, as well as other substituents providing desired properties.

### CROSS-REFERENCE TO RELATED APPLICATIONS

This is an International Application under the Patent Cooperation Treaty, claiming priority to U.S. Provisional Patent Application No. 63/393,035, filed Jul. 28, 2022, the contents of which are incorporated herein by reference in their entirety.

### BACKGROUND

### Field

The present disclosure relates to alkenyl and cyanoalkyl piperidine compounds having activity towards one or more opioid receptors, pharmaceutical compositions containing these compounds, and methods of their use.

### Description of Related Art

Opioid-like analgesics (opiates) are widely used to treat acute and chronic pain in patients, but their use can lead to opioid use disorder (OUD). These therapeutics target at least one of three primary types of endogenous opioid receptors—μ (mu) (MOR), δ (delta) (DOR), and κ (kappa) (KOR) receptors—as well as possible interaction with nociception (NOR) and zeta (ZOR) receptors. Many opiates are mu-opioid agonists that are used for treatment of severe pain due to activation of mu-opioid receptors in the brain and central nervous system (CNS). However, opioid receptors are not limited to CNS, and can be found in other tissues throughout the body. Activation of these “peripheral” receptors by opioid drugs can cause various side effects. For example, opioids can cause nausea and vomiting, as well as inhibition of normal propulsive gastrointestinal function in human and animals resulting in constipation.

Traditional opioid-like analgesics, such as morphine, are full agonists with high efficacy as determined by a functional assay such as the forskolin-induced CAMP Accumulation Assay or the stimulation of [³⁵S]GTPgS Binding at MOR (mu), DOR (delta) and KOR (kappa) opioid receptors. Full efficacy agonists like morphine have been shown to have undesirable effects, such as respiratory depression, constipation, tolerance, and dependence. Compounds that act as partial agonists with less than full efficacy would appear to retain the desired analgesic effects of opioids, while producing only minor or no adverse manifestations. Traditional opioid-like antagonists have been shown to be lifesaving when used to treat opioid overdoses. Illicit opioids have recently become commonplace that are much more potent than heroin. To overcome the respiratory depression, leading to death from overdose of these extremely potent opioids, new more potent antagonists are needed. Thus, there remains a need for analgesics without undesirable side effects, as well as agents for treating those who have developed OUD as a chronic aliment, and new antagonists are needed to help those experiencing an acute overdose of a potent narcotic. Solutions to these technical problems are provided herein.

### BRIEF SUMMARY

A compound having Formula (I), its enantiomer, a racemate thereof, or any combination thereof is provided by the present disclosure.

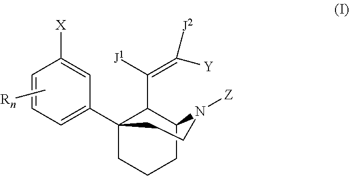

In Formula (I), X is —OR¹, —NR¹R², —CO₂R¹, —CONR¹R², —(CR¹R²)ₘ₁OH, an amide, a nitrogen-containing heteroaryl, or Ar—NHCHO; wherein each R¹ is H, OH, an ether, an ester, a carboxyl, a substituted or unsubstituted C₁-C₃₀ alkyl, a substituted or unsubstituted C₃-C₃₀ cycloalkyl, or a substituted or unsubstituted C₆-C₃₀ aryl; each R² is H, OH, an ether, an ester, a substituted or unsubstituted C₁-C₃₀ alkyl, a substituted or unsubstituted C₃-C₃₀ cycloalkyl, a substituted or unsubstituted C₂-C₃₀ alkanoyl, a substituted or unsubstituted C₄-C₃₀ cycloalkanoyl, or a substituted or unsubstituted C₆-C₃₀ aryl; Ar is a substituted or unsubstituted C₆-C₃₀ aryl; and m1 is an integer of 1 to 10. J¹ and J² are each independently, H or a halogen, or J¹ and Y dependently constitute a bond resulting in a triple bond between their respective carbons. Y is H, methyl, or ethyl, or J¹ and Y dependently constitute a bond resulting in a triple bond between their respective carbons;
and Z is H, -L-W, or —(CR³R⁴)ₘ₂W. L is a substituted or unsubstituted C₂-C₁₀ alkenylene or a substituted or unsubstituted C₂-C₁₀ alkynylene; R³ and R⁴ are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, wherein any two selected from R³ and R⁴ are optionally bonded together to form a ring. W is H, a substituted or unsubstituted C₁-C₃₀ alkyl, a substituted or unsubstituted C₃-C₃₀ cycloalkyl, a substituted or unsubstituted spiro C₃-C₃₀ cycloalkyl. a substituted or unsubstituted C₆-C₃₀ aryl, or a substituted or unsubstituted C₁-C₃₀ heteroaryl; and m2 is an integer of 1 to 10. R is H, a halogen, or a substituted or unsubstituted C₁-C₃₀ alkyl; and n is an integer of 1 to 4. A compound of Formula (I), its enantiomer, a racemate thereof, or any combination thereof is provided wherein J¹ and J² are cis-oriented to each other and trans-oriented to Y and the piperidine. Alternatively, a compound of Formula (I), its enantiomer, a racemate thereof, or any combination thereof is provided wherein J¹ and Y are cis-oriented to each other and Y and the piperidine are trans-oriented to each other.

A compound having Formula (VI), its enantiomer, a racemate thereof, or any combination thereof is provided by the present disclosure.

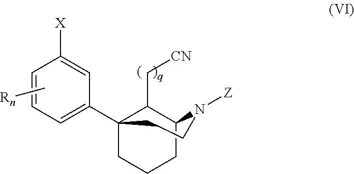

In Formula (VI), X is —OR¹, —NR¹R², —CO₂R¹, —CONR¹R², —(CR¹R²)ₘ₁OH, an amide, a nitrogen-containing heteroaryl, or Ar—NHCHO; wherein each R¹ is H, OH, an ether, an ester, a carboxyl, a substituted or unsubstituted C₁-C₃₀ alkyl, a substituted or unsubstituted C₃-C₃₀ cycloalkyl, or a substituted or unsubstituted C₆-C₃₀ aryl; each R² is H, OH, an ether, an ester, a substituted or unsubstituted C₁-C₃₀ alkyl, a substituted or unsubstituted C₃-C₃₀ cycloalkyl, a substituted or unsubstituted C₂-C₃₀ alkanoyl, a substituted or unsubstituted C₄-C₃₀ cycloalkanoyl, or a substituted or unsubstituted C₆-C₃₀ aryl; Ar is a substituted or unsubstituted C₆-C₃₀ aryl; and m1 is an integer of 1 to 10; q is 0 to 4 carbons; and Z is H, -L-W, or —(CR³R⁴)ₘ₂W. L is a substituted or unsubstituted C₂-C₁₀ alkenylene or a substituted or unsubstituted C₂-C₁₀ alkynylene. R³ and R⁴ are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, wherein any two selected from R³ and R⁴ are optionally bonded together to form a ring. W is H, a substituted or unsubstituted C₁-C₃₀ alkyl, a substituted or unsubstituted C₃-C₃₀ cycloalkyl, a substituted or unsubstituted spiro C₃-C₃₀ cycloalkyl. a substituted or unsubstituted C₆-C₃₀ aryl, or a substituted or unsubstituted C₁-C₃₀ heteroaryl; and m2 is an integer of 1 to 10. R is H, a halogen, or a substituted or unsubstituted C₁-C₃₀ alkyl; and n is an integer of 1 to 4.

A compound of Formula (I) or Formula (VI), its enantiomer, a racemate thereof, or any combination thereof, comprising a full agonist, a partial agonist, or an antagonist, or any combination thereof of a mu-opioid receptor is provided. A compound of Formula (I) or Formula (VI), its enantiomer, a racemate thereof, or any combination thereof, that also lacks agonist activity fully or partially against a kappa-opioid receptor, or a delta-opioid receptor, or both is provided. A pharmaceutical composition is provided that can comprise a therapeutically effective amount of a compound of Formula (I) or Formula (VI), its enantiomer, a racemate thereof, or any combination thereof, together with a pharmaceutically acceptable carrier.

A method of prevention or treatment of pain is provided that can comprise administering to a patient in need thereof a composition comprising a therapeutically effective amount of a compound of Formula (I) or Formula (VI), its enantiomer, a racemate thereof, or any combination thereof optionally in combination with one or more additional active ingredients. A method of treating or preventing an opioid use disorder (OUD) is provided that can comprise administering to a patient in need thereof a composition comprising a therapeutically effective amount of a compound of Formula (I) or Formula (VI), its enantiomer, a racemate thereof, or any combination thereof optionally in combination with one or more additional active ingredients. A method of treating or preventing an opioid overdose is also provided that comprise administering to a patient in need thereof a composition comprising a therapeutically effective amount of a compound of Formula (I) or Formula (VI), its enantiomer, a racemate thereof, or any combination thereof optionally in combination with one or more additional active ingredients.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1A depicts a scheme for synthesizing Compound 4 in accordance with the present disclosure.

FIG. 1B depicts a scheme for synthesizing Compounds 5 and 6 in accordance with the present disclosure.

FIG. 1C depicts a scheme for synthesizing Compounds 8 and 9 in accordance with the present disclosure.

FIG. 2 depicts a scheme for synthesizing Compounds 11 and 12 in accordance with the present disclosure.

FIG. 3A depicts a scheme for synthesizing Compounds 13 and 14 in accordance with the present disclosure.

FIG. 3B depicts an X-ray crystal structure of Compound 14 in accordance with the present disclosure.

FIG. 4 depicts a scheme for synthesizing Compounds 18 and 19 in accordance with the present disclosure.

FIG. 5 depicts a scheme for synthesizing Compounds 22 and 23 in accordance with the present disclosure.

FIG. 6 depicts a scheme for synthesizing Compounds 26 and 27 in accordance with the present disclosure.

FIG. 7 depicts a scheme for synthesizing Compounds 29 and 30 in accordance with the present disclosure.

FIG. 8 depicts a scheme for synthesizing Compounds 33 and 34 in accordance with the present disclosure.

FIG. 9 depicts a scheme for synthesizing Compound 39 in accordance with the present disclosure.

FIG. 10 depicts a scheme for synthesizing Compound 41 in accordance with the present disclosure.

FIG. 11 depicts a scheme for synthesizing Compound 47 in accordance with the present disclosure.

### DETAILED DESCRIPTION

Compounds are described using standard nomenclature. Unless defined otherwise, all technical and scientific terms used herein have the same meaning as is commonly understood by one of skill in the art to which this disclosure belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (for example, “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, for example, in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein can include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I, and ¹³¹I. Formulae, subformulae thereof, and compounds thereof include all pharmaceutically acceptable salts of the same.

The term “substituted” means that any one or more hydrogens on the designated atom or group is replaced with a selection from the indicated group, provided that the designated atom's normal valence is not exceeded. Combinations of substituents and/or variables are permissible, for example, if such combinations result in stable compounds or useful synthetic intermediates. A stable compound or stable structure can be a compound that is sufficiently robust to survive isolation from a reaction mixture, and subsequent formulation into an effective therapeutic agent. A substituent or combination of substituents described with respect to one formula, subformula, or compound, can also be used in any other formula, subformula, or compound where consistent with valence, polarity, size, structure, and other parameters, unless otherwise indicated. Any substituent or combination of substituents described herein with respect to a particular atom or atoms can also be excluded as an option for replacing one or more hydrogens at the particular atom, atoms, or subset thereof.

A dash (“-”) that is not between two letters or symbols is used to indicate a point of attachment for a substituent.

“Alkyl” refers to a group derived from a straight or branched chain saturated aliphatic hydrocarbon having the specified number of carbon atoms and having a valence of one, optionally substituted with one or more substituents where indicated, provided that the valence of the alkyl group is not exceeded.

“Cycloalkyl” refers to a group that comprises one or more saturated and/or partially saturated rings in which all ring members are carbon, the group having the specified number of carbon atoms. Cycloalkyl groups do not include an aromatic ring or a heterocyclic ring.

“Alkanoyl” refers to a group having formula “alkyl-C(═O)—”, wherein “alkyl” is the same as defined above.

“Cycloalkanoyl” refers to a group having formula “cycloalkyl-C(═O)—”, wherein “cycloalkyl” is the same as defined above.

“Aryl” refers to a cyclic group in which all ring members are carbon and all rings are aromatic, the group having the specified number of carbon atoms, and having a valence of one, optionally substituted with one or more substituents where indicated, provided that the valence of the aryl group is not exceeded. More than one ring can be present, and any additional rings can be fused, pendant, spirocyclic, or a combination thereof.

“Heteroaryl” means a monovalent carbocyclic ring group that includes one or more aromatic rings, in which at least one ring member (for example, one, two or three ring members) is a heteroatom selected from nitrogen (N), oxygen (O), sulfur(S), and phosphorus (P), the group having the specified number of carbon atoms.

“Halogen” means fluoro, chloro, bromo, or iodo, and are defined herein to include all isotopes of the same, including heavy isotopes and radioactive isotopes. Examples of useful halo isotopes include ¹⁸F, ⁷⁶Br, and ¹³¹I. Additional isotopes will be readily appreciated by one of skill in the art.

“Substituted” means that the compound or group is substituted with at least one (for example, 1, 2, 3, or 4) substituent independently selected from a halogen (—F, Cl, —Br, —I), a hydroxyl (—OH), a C₁-C₉ alkoxy, a C₁-C₉ haloalkoxy, an oxo (═O), a nitro (—NO₂), a cyano (—CN), an amino (—NR², wherein each R is independently hydrogen or C₁-C₁₀ alkyl), an azido (—N₃), an amidino (—C(═NH)NH₂), a hydrazino (—NHNH₂), a hydrazono (—C(═NNH₂)—), a carbonyl (—C(═O)—), a carbamoyl group (—C(O)NH₂), a sulfonyl (—S(═O)₂—), a thiol (—SH), a thiocyano (—SCN), a tosyl (CH₃C₆H₄SO₂—), a carboxylic acid (—C(═O) OH), a carboxylic C₁-C₆ alkyl ester (—C(═O) OR wherein R is C₁-C₁₀ alkyl), a carboxylic acid salt (—C(═O)OM) wherein M is an organic or inorganic anion, a sulfonic acid (—SO₃H₂), a sulfonic mono- or dibasic salt (—SO₃MH or —SO₃M₂ wherein M is an organic or inorganic anion), a phosphoric acid (—PO₃H₂), a phosphoric acid mono- or dibasic salt (—PO₃MH or —PO₃M₂ wherein M is an organic or inorganic anion), a C₁-C₁₂ alkyl, a C₃-C₁₂ cycloalkyl, a C₂-C₁₂ alkenyl, a C₅-C₁₂ cycloalkenyl, a C₂-C₁₂ alkynyl, a C₆-C₁₂ aryl, a C₇-C₁₃ arylalkylene, a C₄-C₁₂ heterocycloalkyl, and a C₃-C₁₂ heteroaryl instead of hydrogen, provided that the substituted atom's normal valence is not exceeded.

“Pharmaceutical composition” means a composition comprising at least one active agent, such as a compound or salt of Formula (I), and at least one other substance, such as a carrier. Pharmaceutical compositions can meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, for example, excipient, diluent, or vehicle, that is useful in preparing a pharmaceutical composition that is generally safe, non-toxic and neither biologically nor otherwise undesirable, and can include a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. For example, the patient can be a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. Treatment of the disease can be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

Compounds of formulae can contain one or more asymmetric elements such as stereogenic centers, stereogenic axes and the like, for example, asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent can be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, for example, melting points, boiling points, spectral properties, and reactivities. Mixtures of diastereomers can separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which can occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture can occur where there has been no stereoselection or stereospecificity in a chemical reaction or process. Combinations of two enantiomeric species other than 50:50 racemic mixtures are also provided by the present disclosure, for example, 1:10,000, 1:1,000, 1:100, 1:10, 1:9, 1:7.5, 1:5, 1:3, 1:2.5, 1:2, or 1:1.5, or any opposite ratio, or any intervening ratio.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions can be carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids, for example, hydrochloric, hydrobromic, sulfuric, sulfamic, phosphoric, nitric and the like; and the salts prepared from organic acids, for example, acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂) n-COOH where n is 0-4, and the like. Lists of additional suitable salts can be found, for example, in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

A compound having Formula (I), its enantiomer, a racemate thereof, or any combination thereof is provided by the present disclosure.

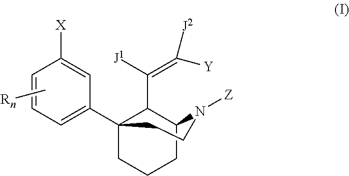

In Formula (I), X is —OR¹, —NR¹R², —CO₂R¹, —CONR¹R², —(CR¹R²)ₘ₁OH, an amide, a nitrogen-containing heteroaryl, or Ar—NHCHO; wherein each R¹ is H, OH, an ether, an ester, a carboxyl, a substituted or unsubstituted C₁-C₃₀ alkyl, a substituted or unsubstituted C₃-C₃₀ cycloalkyl, or a substituted or unsubstituted C₆-C₃₀ aryl; each R² is H, OH, an ether, an ester, a substituted or unsubstituted C₁-C₃₀ alkyl, a substituted or unsubstituted C₃-C₃₀ cycloalkyl, a substituted or unsubstituted C₂-C₃₀ alkanoyl, a substituted or unsubstituted C₄-C₃₀ cycloalkanoyl, or a substituted or unsubstituted C₆-C₃₀ aryl; Ar is a substituted or unsubstituted C₆-C₃₀ aryl; and m1 is an integer of 1 to 10. J¹ and J² are each independently, H or a halogen, or J¹ and Y dependently constitute a bond resulting in a triple bond between their respective carbons. Y is H, methyl, or ethyl, or J¹ and Y dependently constitute a bond resulting in a triple bond between their respective carbons;
and Z is H, -L-W, or —(CR³R⁴)ₘ₂W. L is a substituted or unsubstituted C₂-C₁₀ alkenylene or a substituted or unsubstituted C₂-C₁₀ alkynylene; R³ and R⁴ are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, wherein any two selected from R³ and R⁴ are optionally bonded together to form a ring. W is H, a substituted or unsubstituted C₁-C₃₀ alkyl, a substituted or unsubstituted C₃-C₃₀ cycloalkyl, a substituted or unsubstituted spiro C₃-C₃₀ cycloalkyl. a substituted or unsubstituted C₆-C₃₀ aryl, or a substituted or unsubstituted C₁-C₃₀ heteroaryl; and m2 is an integer of 1 to 10. R is H, a halogen, or a substituted or unsubstituted C₁-C₃₀ alkyl; and n is an integer of 1 to 4. A compound of Formula (I), its enantiomer, a racemate thereof, or any combination thereof is provided wherein J¹ and J² are cis-oriented to each other and trans-oriented to Y and the piperidine. Alternatively, a compound of Formula (I), its enantiomer, a racemate thereof, or any combination thereof is provided wherein J¹ and Y are cis-oriented to each other and Y and the piperidine are trans-oriented to each other.

The “enantiomer” of the compound having Formula (I) refers to a compound having Formula (I-ent) illustrated below:

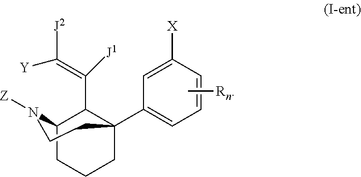

A compound of Formula (Ia), its enantiomer, a racemate thereof, or any combination thereof is provided:

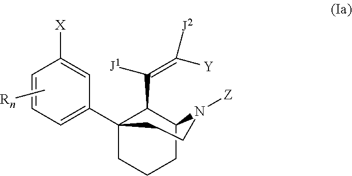

wherein in Formula (Ia), R, X, J¹, J², Y, Z, and n are the same as in Formula I.

The “enantiomer” of the compound having Formula (Ia) refers to a compound having Formula (la-ent) illustrated below:

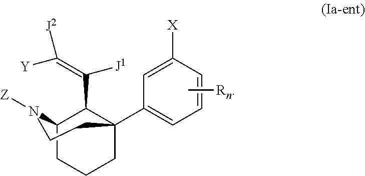

A compound of Formula (Ib), its enantiomer, a racemate thereof, or any combination thereof is provided:

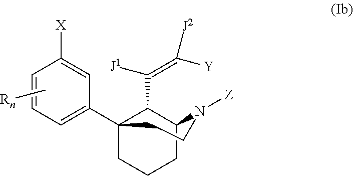

wherein in Formula (Ib), R, X, J¹, J², Y, Z, and n are the same as in Formula I.

The “enantiomer” of the compound having Formula (Ib) refers to a compound having Formula (Ib-ent) illustrated below:

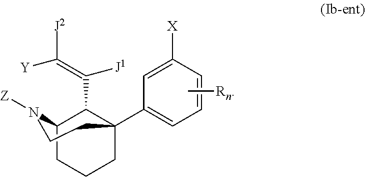

A compound of Formula (I), its enantiomer, a racemate thereof, or any combination thereof is provided wherein X is OH, NH₂, or NHR⁵, and R⁵ is CHO or —C(O) OR⁶, and each R⁶ is Hora substituted or unsubstituted C₁-C₃₀ alkyl. A compound of Formula (I), its enantiomer, a racemate thereof, or any combination thereof is provided wherein Z is —(CR³R⁴)ₘ₂W, R³ and R⁴ are each H, W is a substituted or unsubstituted C₆-C₃₀ aryl, and m2 is an integer of 1 to 5. A compound of Formula (I), its enantiomer, a racemate thereof, or any combination thereof is provided wherein Z is —CH₂CH₂W, wherein Z is —CH₂CH₂W, and W is unsubstituted C₆-C₃₀ aryl, C₆-C₃₀ aryl substituted with —F, —Cl, —Br, —OH, —NH₂ or —NO₂, unsubstituted C₁-C₃₀ heteroaryl, or C₁-C₃₀ heteroaryl substituted with —F, —Cl, —Br, —OH, —NH₂ or —NO₂.

A compound having Formula (II), its enantiomer, a racemate thereof, or any combination thereof is provided:

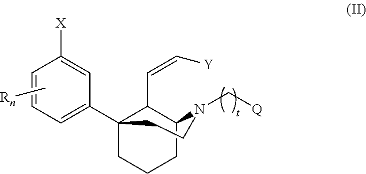

wherein in Formula (II), R, X, Y, and n are the same as in Formula I, Q is alkyl, alkenyl, cycloalkyl, cyclopropyl, cyclopentyl, heterocyclic alkyl, bicyclic cycloalkyl, bicyclic heterocyclic alkyl, phenyl, amine, cyclic amine, piperidine, pyrrolidine, pyrrole, heterocyclic amine, bicyclic heterocyclic amine, indole, azaspirononane, spirodecane, substituted or unsubstituted C₆-C₃₀ aryl, substituted or unsubstituted C₆-C₃₀ heteroaryl, and t is 0-4 carbons.

The “enantiomer” of the compound having Formula (II) refers to a compound having Formula (II-ent) illustrated below:

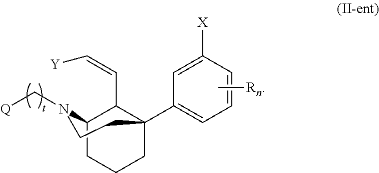

A compound of Formula (IIa), its enantiomer, a racemate thereof, or any combination thereof is provided:

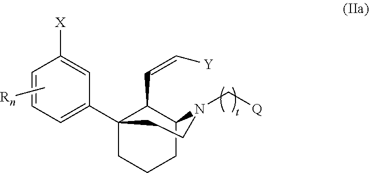

wherein in Formula (IIa), the substituents are the same as in Formula (II).

The “enantiomer” of the compound having Formula (IIa) refers to a compound having Formula (IIa-ent) illustrated below:

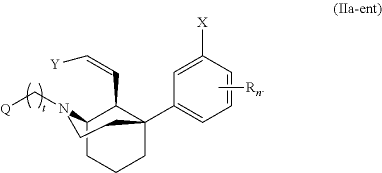

A compound of Formula (IIb), its enantiomer, a racemate thereof, or any combination thereof is provided:

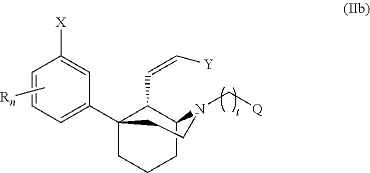

wherein in Formula (IIb), the substituents are the same as in Formula (II).

The “enantiomer” of the compound having Formula (IIb) refers to a compound having Formula (IIb-ent) illustrated below:

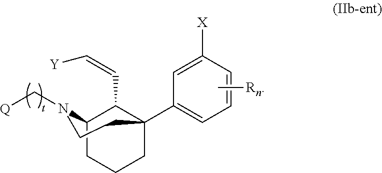

A compound having Formula (III), its enantiomer, a racemate thereof, or any combination thereof is provided:

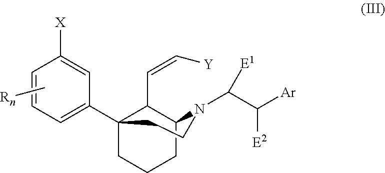

wherein in Formula (III), R, Y, and n are the same as in Formula I, Ar is a substituted or unsubstituted C₆-C₃₀ aryl, and X is OH, NH₂, or NHR⁵, R⁵ is CHO or —C(O) OR⁶, each R⁶ is H or a substituted or unsubstituted C₁-C₃₀ alkyl, E¹ and E² are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, and any two selected from R³ and R⁴ are optionally bonded together to form a ring.

The “enantiomer” of the compound having Formula (III) refers to a compound having Formula (III-ent) illustrated below:

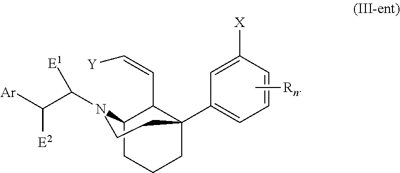

A compound of Formula (IIIa), its enantiomer, a racemate thereof, or any combination thereof is provided:

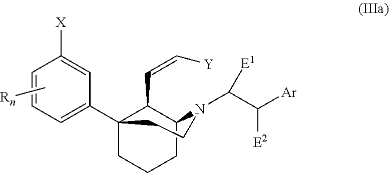

wherein in Formula (IIIa), the substituents are the same as in Formula (III).

The “enantiomer” of the compound having Formula (IIIa) refers to a compound having Formula (IIIa-ent) illustrated below:

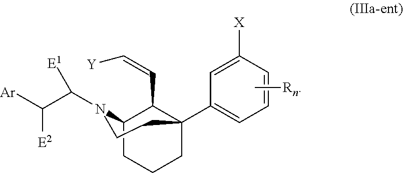

A compound of Formula (IIIb), its enantiomer, a racemate thereof, or any combination thereof is provided:

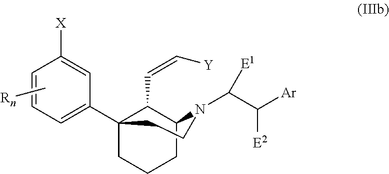

wherein in Formula (IIIb), the substituents are the same as in Formula (III).

The “enantiomer” of the compound having Formula (IIIb) refers to a compound having Formula (IIIb-ent) illustrated below:

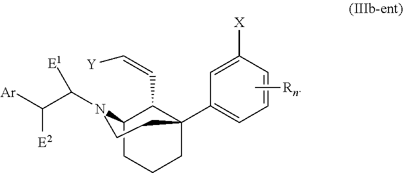

A compound of Formula (I), its enantiomer, a racemate thereof, or any combination thereof is provided wherein X is an amide or a nitrogen-containing heteroaryl. A compound of Formula (I), its enantiomer, a racemate thereof, or any combination thereof is provided wherein X is or comprises one or more of pyrrole, imidazole, a triazole, tetrazole, pyridine, pyrimidine, pyrazaine, triazine, pyridazine, a quinoline, a purine, a pteridine, an acridine, and an alloxane. A compound of Formula (I), its enantiomer, a racemate thereof, or any combination thereof is provided wherein X is imidazole or formanilide. A compound of Formula (I), its enantiomer, a racemate thereof, or any combination thereof is provided wherein R is H or halogen.

A compound having Formula (IV), its enantiomer, a racemate thereof, or any combination thereof is provided:

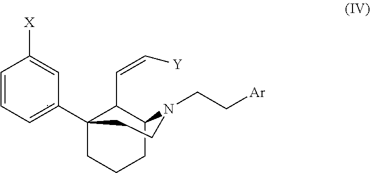

wherein in Formula (IV), Y is the same as in Formula I, Ar is a substituted or unsubstituted C₆-C₃₀ aryl, X is OH, NH₂, or NHR⁵, R⁵ is CHO or —C(O) OR⁶, and each R⁶ is H or a substituted or unsubstituted C₁-C₃₀ alkyl.

The “enantiomer” of the compound having Formula (IV) refers to a compound having Formula (IV-ent) illustrated below:

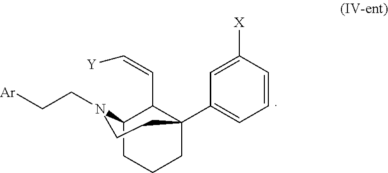

A compound of Formula (IVa), its enantiomer, a racemate thereof, or any combination thereof is provided:

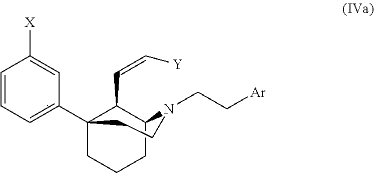

wherein in Formula (IVa), the substituents are the same as in Formula (IVa).

The “enantiomer” of the compound having Formula (IVa) refers to a compound having Formula (IVa-ent) illustrated below:

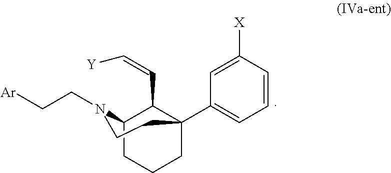

A compound of Formula (IVb), its enantiomer, a racemate thereof, or any combination thereof is provided:

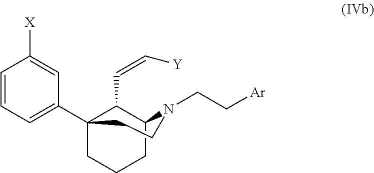

wherein in Formula (IVb), the substituents are the same as in Formula (IV).

The “enantiomer” of the compound having Formula (IVb) refers to a compound having Formula (IVb-ent) illustrated below:

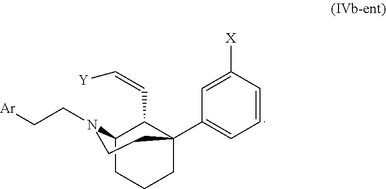

A compound having Formula (V), its enantiomer, a racemate thereof, or any combination thereof is provided:

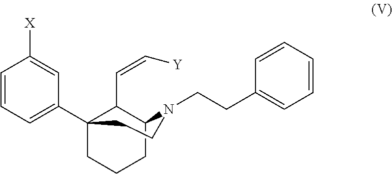

wherein in Formula (V), Y is the same as in Formula I, and X is OH, NH₂, or NHR⁵, R⁵ is CHO or —C(O) OR⁶, and each R⁶ is H or a substituted or unsubstituted C₁-C₃₀ alkyl.

The “enantiomer” of the compound having Formula (V) refers to a compound having Formula (V-ent) illustrated below:

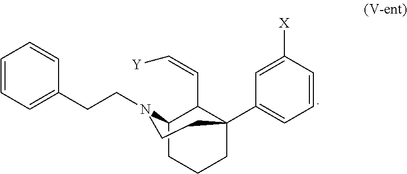

A compound of Formula (Va), its enantiomer, a racemate thereof, or any combination thereof is provided:

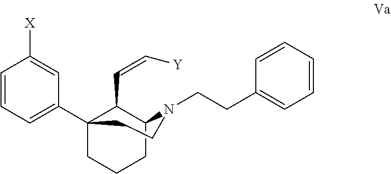

wherein in Formula (Va), the substituents are the same as in Formula (V).

The “enantiomer” of the compound having Formula (Va) refers to a compound having Formula (Va-ent) illustrated below:

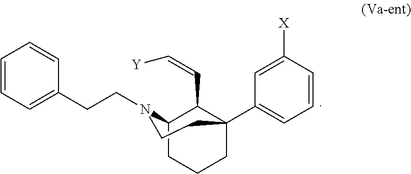

A compound of Formula (Vb), its enantiomer, a racemate thereof, or any combination thereof is provided:

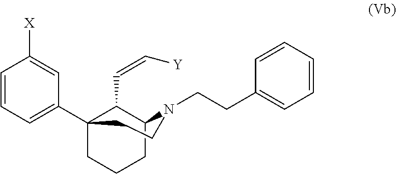

wherein in Formula (Vb), the substituents are the same as in Formula (V).

The “enantiomer” of the compound having Formula (Vb) refers to a compound having Formula (Vb-ent) illustrated below:

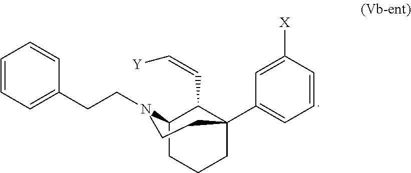

A compound having Formula (XI), its enantiomer, a racemate thereof, or any combination thereof is provided:

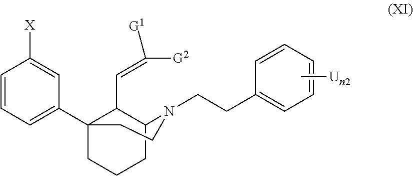

wherein in Formula (XI), X, and n are the same as in Formula I, G¹ and G² are independently H or a halogen, X is OH, NH₂, or NHR⁵, R⁵ is CHO or —C(O) OR⁶, and each R⁶ is H or a substituted or unsubstituted C₁-C₃₀ alkyl U is a H, a halogen, or a substituted or unsubstituted C₁-C₃₀ alkyl; and n2 is an integer of 1 to 4.

A compound having Formula (XII), its enantiomer, a racemate thereof, or any combination thereof is provided:

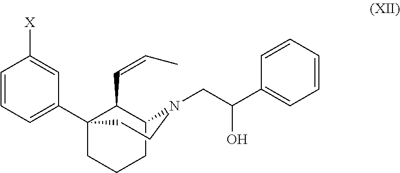

wherein in Formula (XII), X is OH, NH₂, or NHR⁵, R⁵ is CHO or —C(O) OR⁶, and each R⁶ is H or a substituted or unsubstituted C₁-C₃₀ alkyl.

A compound having Formula (XIII), its enantiomer, a racemate thereof, or any combination thereof is provided:

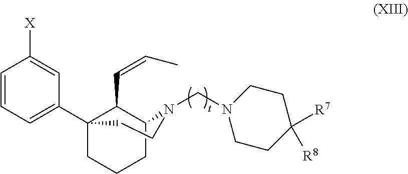

wherein in Formula (XIII), t is 0-4 carbons, X is OH, NH₂, or NHR⁵, R⁵ is CHO or —C(O) OR⁶, each R⁶ is Hora substituted or unsubstituted C₁-C₃₀ alkyl, and R⁷ and R⁸ are hydrogen, or R⁷ and R⁸ are dependently a C₁-C₆ unsubstituted heterocycloalkyl, a C₁-C₆ substituted heterocycloalkyl, an unsubstituted spiro C₃-C₃₀ heterocycloalkyl, a substituted spiro C₃-C₃₀ heterocycloalkyl, a C₁-C₆ unsubstituted heteroaryl, or a C₁-C₆ substituted heteroaryl.

A compound having Formula (XIV), its enantiomer, a racemate thereof, or any combination thereof is provided:

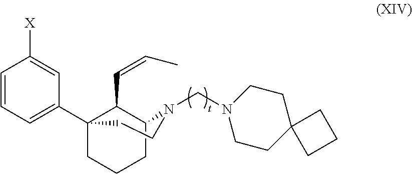

wherein in Formula (XIV), t is 0-4 carbons, and X is OH, NH₂, or NHR⁵, R⁵ is CHO or —C(O) OR⁶, and each R⁶ is H or a substituted or unsubstituted C₁-C₃₀ alkyl.

A compound having Formula (XV), its enantiomer, a racemate thereof, or any combination thereof is provided:

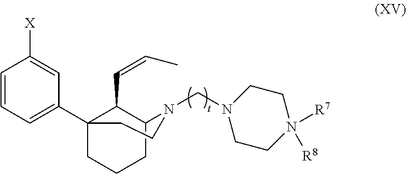

wherein in Formula (XV), t is 0-4 carbons, X is OH, NH₂, or NHR⁵, R⁵ is CHO or —C(O) OR⁶, each R⁶ is H or a substituted or unsubstituted C₁-C₃₀ alkyl, and R⁷ and R⁸ are hydrogen, or R⁷ and R⁸ are dependently a C₁-C₆ unsubstituted heterocycloalkyl, a C₁-C₆ substituted heterocycloalkyl, an unsubstituted spiro C₃-C₃₀ heterocycloalkyl, a substituted spiro C₃-C₃₀ heterocycloalkyl, a C₁-C₆ unsubstituted heteroaryl, or a C₁-C₆ substituted heteroaryl.

A compound having Formula (XVI), its enantiomer, a racemate thereof, or any combination thereof is provided:

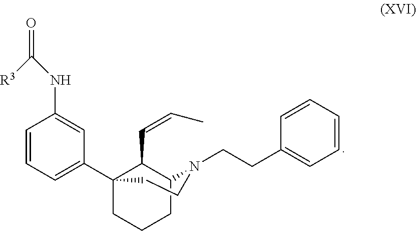

wherein in Formula (XVI), R³ is the same as in Formula I.

A compound having Formula (XVII), its enantiomer, a racemate thereof, or any combination thereof is provided:

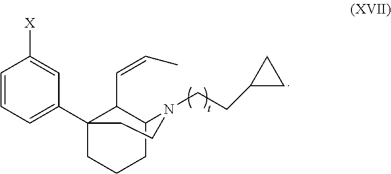

wherein in Formula (XVII), t is 0-4 carbons, and X is OH, NH₂, or NHR⁵, R⁵ is CHO or —C(O) OR⁶, and each R⁶ is H or a substituted or unsubstituted C₁-C₃₀ alkyl.

A compound having Formula (XVIII), its enantiomer, a racemate thereof, or any combination thereof is provided:

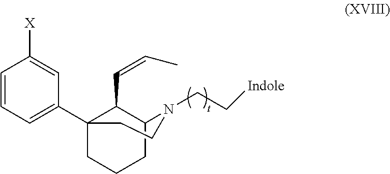

wherein in Formula (XVIII), t is 0-4 carbons, and X is OH, NH₂, or NHR⁵, R⁵ is CHO or —C(O) OR⁶, wherein each R⁶ is H or a substituted or unsubstituted C₁-C₃₀ alkyl.

A compound having Formula (VI), its enantiomer, a racemate thereof, or any combination thereof is provided by the present disclosure.

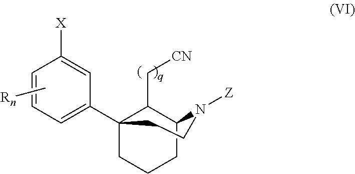

In Formula (VI), X is —OR¹, —NR¹R², —CO₂R¹, —CONR¹R², —(CR¹R²)ₘ₁OH, an amide, a nitrogen-containing heteroaryl, or Ar—NHCHO; wherein each R¹ is H, OH, an ether, an ester, a carboxyl, a substituted or unsubstituted C₁-C₃₀ alkyl, a substituted or unsubstituted C₃-C₃₀ cycloalkyl, or a substituted or unsubstituted C₆-C₃₀ aryl; each R² is H, OH, an ether, an ester, a substituted or unsubstituted C₁-C₃₀ alkyl, a substituted or unsubstituted C₃-C₃₀ cycloalkyl, a substituted or unsubstituted C₂-C₃₀ alkanoyl, a substituted or unsubstituted C₄-C₃₀ cycloalkanoyl, or a substituted or unsubstituted C₆-C₃₀ aryl; Ar is a substituted or unsubstituted C₆-C₃₀ aryl; and m1 is an integer of 1 to 10; q is 0 to 4 carbons; and Z is H, -L-W, or —(CR³R⁴)ₘ₂W. L is a substituted or unsubstituted C₂-C₁₀ alkenylene or a substituted or unsubstituted C₂-C₁₀ alkynylene. R³ and R⁴ are each independently H, OH, O (ketone), a halogen, an amine, a substituted or unsubstituted C₁-C₃₀ alkyl, a substituted or unsubstituted C₃-C₃ cycloalkyl, a substituted or unsubstituted C₆-C₃₀ aryl, or a substituted or unsubstituted C₁-C₃₀ heteroaryl, wherein any two selected from R³ and R⁴ are optionally bonded together to form a ring. W is H, a substituted or unsubstituted C₁-C₃₀ alkyl, a substituted or unsubstituted C₃-C₃₀ cycloalkyl, a substituted or unsubstituted spiro C₃-C₃₀ cycloalkyl. a substituted or unsubstituted C₆-C₃₀ aryl, or a substituted or unsubstituted C₁-C₃₀ heteroaryl; and m2 is an integer of 1 to 10. R is H, a halogen, or a substituted or unsubstituted C₁-C₃₀ alkyl; and n is an integer of 1 to 4.

The “enantiomer” of the compound having Formula (VI) refers to a compound having Formula (VI-ent) illustrated below:

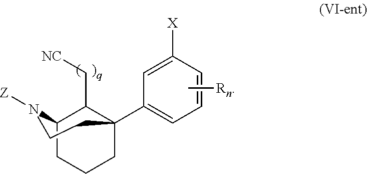

A compound of Formula (VIa), its enantiomer, a racemate thereof, or any combination thereof is provided:

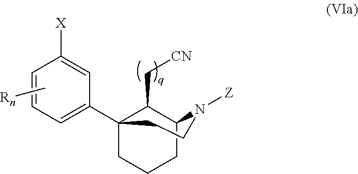

wherein in Formula (VIa), R, X, Y, Z, and n are the same as in Formula (VI).

The “enantiomer” of the compound having Formula (VIa) refers to a compound having Formula (VIa-ent) illustrated below:

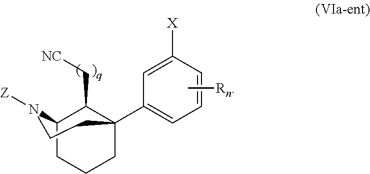

A compound of Formula (VIb), its enantiomer, a racemate thereof, or any combination thereof is provided:

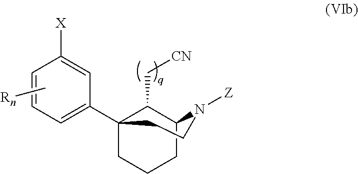

wherein in Formula (VIb), R, X, Z, and n are the same as in any preceding or following embodiment/feature/aspect.

The “enantiomer” of the compound having Formula (VIb) refers to a compound having Formula (VIb-ent) illustrated below:

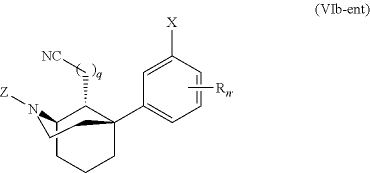

A compound of Formula (VI), its enantiomer, a racemate thereof, or any combination thereof is provided wherein X is OH, NH₂, or NHR⁵, R⁵ is CHO or —C(O) OR⁶, and each R⁶ is Hora substituted or unsubstituted C₁-C₃₀ alkyl. A compound of Formula (VI), its enantiomer, a racemate thereof, or any combination thereof is provided wherein Z is —(CR⁷R⁸)ₘ₂W, R³ and R⁴ are each H, W is a substituted or unsubstituted C₆-C₃₀ aryl, and m2 is an integer of 1 to 5. A compound of Formula (VI), its enantiomer, a racemate thereof, or any combination thereof is provided wherein Z is —CH₂CH₂W, and W is unsubstituted C₆-C₃₀ aryl, C₆-C₃₀ aryl substituted with —F, —Cl, —Br, —OH, —NH₂ or —NO₂, unsubstituted C₁-C₃₀ heteroaryl, or C₁-C₃₀ heteroaryl substituted with —F, —Cl, —Br, —OH, —NH₂ or —NO₂.

A compound having Formula (VII), its enantiomer, a racemate thereof, or any combination thereof is provided:

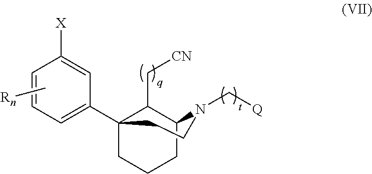

wherein in Formula (VII), R, X, q, and n are the same as in Formula (VI), Q is alkyl, alkenyl, cycloalkyl, cyclopropyl, cyclopentyl, heterocyclic alkyl, bicyclic cycloalkyl, bicyclic heterocyclic alkyl, phenyl, amine, cyclic amine, piperidine, pyrrolidine, pyrrole, heterocyclic amine, bicyclic heterocyclic amine, indole, azaspirononane, spirodecane, substituted or unsubstituted C₆-C₃₀ aryl, substituted or unsubstituted C₆-C₃₀ heteroaryl, and t is 0-4 carbons.

The “enantiomer” of the compound having Formula (VII) refers to a compound having Formula (VII-ent) illustrated below:

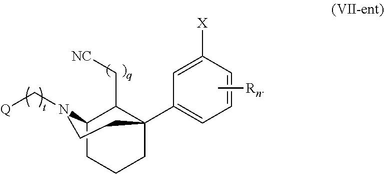

A compound of Formula (VIIa), its enantiomer, a racemate thereof, or any combination thereof is provided:

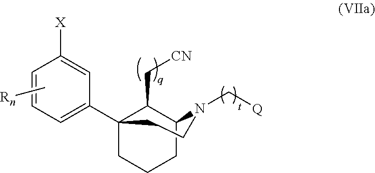

wherein in Formula (VIIa), the substituents are the same as in Formula (VII).

The “enantiomer” of the compound having Formula (VIIa) refers to a compound having Formula (VIIa-ent) illustrated below:

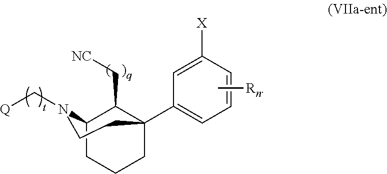

A compound of Formula (VIIb), its enantiomer, a racemate thereof, or any combination thereof is provided:

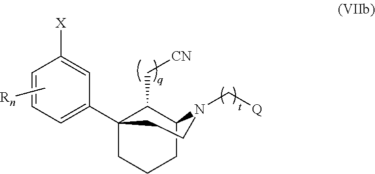

wherein in Formula (VIIb), the substituents are the same as in Formula (VII).

The “enantiomer” of the compound having Formula (VIIb) refers to a compound having Formula (VIIb-ent) illustrated below:

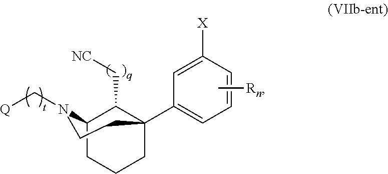

A compound having Formula (VIII), its enantiomer, a racemate thereof, or any combination thereof is provided:

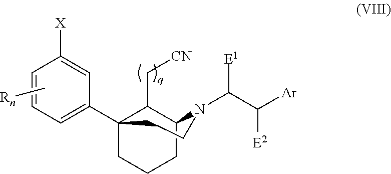

wherein in Formula (VIII), R, q, and n are the same as in Formula (VI), Ar is a substituted or unsubstituted C₆-C₃₀ aryl, and X is OH, NH₂, or NHR⁵, R⁵ is CHO or —C(O) OR⁶, wherein each R⁶ is H or a substituted or unsubstituted C₁-C₃₀ alkyl, and E¹ and E² are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, and any two selected from R³ and R⁴ are optionally bonded together to form a ring.

The “enantiomer” of the compound having Formula (VIII) refers to a compound having Formula (VIII-ent) illustrated below:

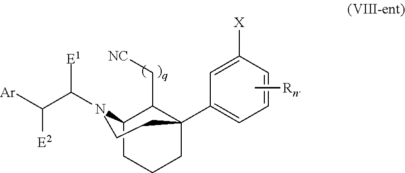

A compound of Formula (VIIIa), its enantiomer, a racemate thereof, or any combination thereof is provided:

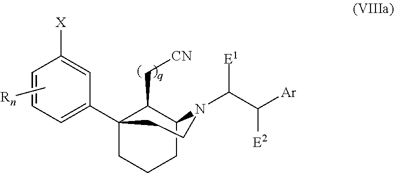

wherein in Formula (VIIIa), the substituents are the same as in Formula (VIII).

The “enantiomer” of the compound having Formula (VIIIa) refers to a compound having Formula (VIIIa-ent) illustrated below:

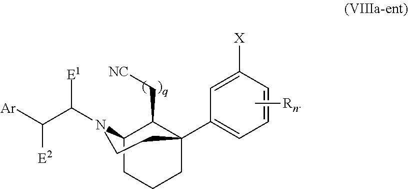

A compound of Formula (VIIIb), its enantiomer, a racemate thereof, or any combination thereof is provided:

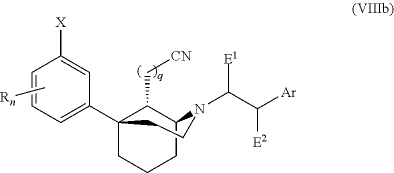

wherein in Formula (VIIIb), the substituents are the same as in Formula (VIII).

The “enantiomer” of the compound having Formula (VIIIb) refers to a compound having Formula (VIIIb-ent) illustrated below:

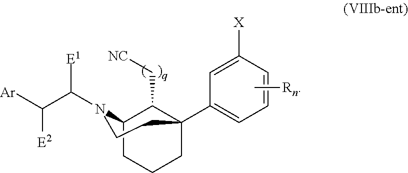

A compound of Formula (VI), its enantiomer, a racemate thereof, or any combination thereof is provided wherein X is an amide or a nitrogen-containing heteroaryl. A compound of Formula (VI), its enantiomer, a racemate thereof, or any combination thereof is provided wherein X is or comprises one or more of pyrrole, imidazole, a triazole, tetrazole, pyridine, pyrimidine, pyrazaine, triazine, pyridazine, a quinoline, a purine, a pteridine, an acridine, and an alloxane. A compound of Formula (VI), its enantiomer, a racemate thereof, or any combination thereof is provided wherein X is imidazole or formanilide. A compound of Formula (VI), its enantiomer, a racemate thereof, or any combination thereof is provided wherein R is H or halogen.

A compound having Formula (IX), its enantiomer, a racemate thereof, or any combination thereof is provided:

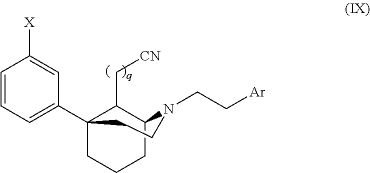

wherein in Formula (IX), q is the same as in Formula I, Ar is a substituted or unsubstituted C₆-C₃₀ aryl, and X is OH, NH₂, or NHR⁵, R⁵ is CHO or —C(O) OR⁶, and each R⁶ is H or a substituted or unsubstituted C₁-C₃₀ alkyl.

The “enantiomer” of the compound having Formula (IX) refers to a compound having Formula (IX-ent) illustrated below:

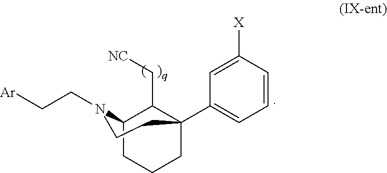

A compound of Formula (IXa), its enantiomer, a racemate thereof, or any combination thereof is provided:

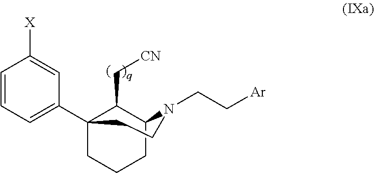

wherein in Formula (IXa), the substituents are the same as in Formula (IX).

The “enantiomer” of the compound having Formula (IXa) refers to a compound having Formula (IXa-ent) illustrated below:

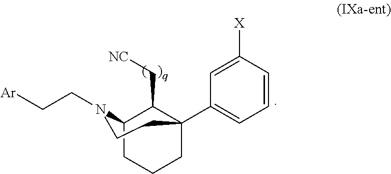

A compound of Formula (IXb), its enantiomer, a racemate thereof, or any combination thereof is provided:

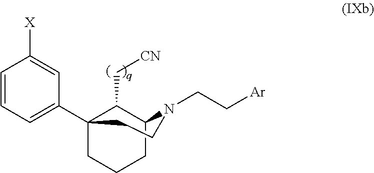

wherein in Formula (IXb), the substituents are the same as in Formula (IX).

The “enantiomer” of the compound having Formula (IXb) refers to a compound having Formula (IXb-ent) illustrated below:

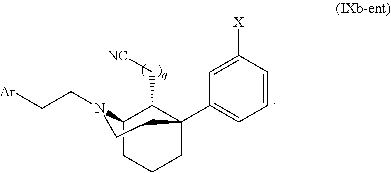

A compound having Formula (X), its enantiomer, a racemate thereof, or any combination thereof is provided:

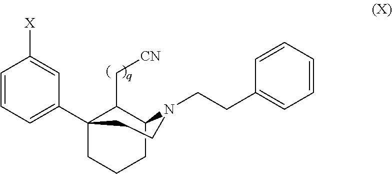

wherein in Formula (X), q is the same as in Formula (VI), and X is OH, NH₂, or NHR⁵, R⁵ is CHO or —C(O) OR⁶, and each R⁶ is H or a substituted or unsubstituted C₁-C₃₀ alkyl.

The “enantiomer” of the compound having Formula (X) refers to a compound having Formula (X-ent) illustrated below:

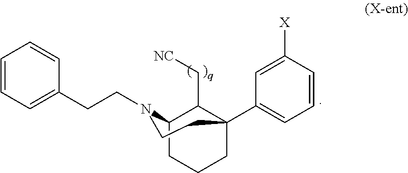

A compound of Formula (Xa), its enantiomer, a racemate thereof, or any combination thereof is provided:

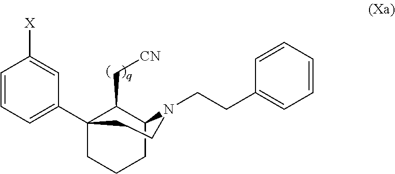

wherein in Formula (Xa), the substituents are the same as in Formula (X).

The “enantiomer” of the compound having Formula (Xa) refers to a compound having Formula (Xa-ent) illustrated below:

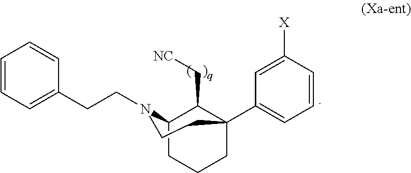

A compound of Formula (Xb), its enantiomer, a racemate thereof, or any combination thereof is provided:

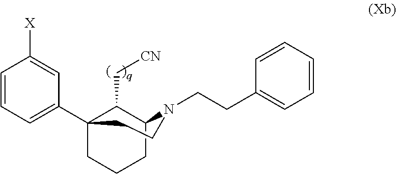

wherein in Formula (Xb), the substituents are the same as in Formula (X).

The “enantiomer” of the compound having Formula (Xb) refers to a compound having Formula (Xb-ent) illustrated below:

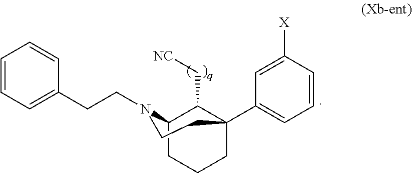

A compound having Formula (XIX), its enantiomer, a racemate thereof, or any combination thereof is provided:

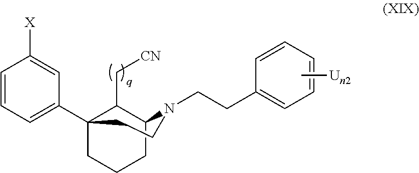

wherein in Formula (XIX), q is the same as in Formula (VI), and U is a H, a halogen, or a substituted or unsubstituted C₁-C₃₀ alkyl; and n2 is an integer of 1 to 4.

A compound of Formula (I) or Formula (VI), its enantiomer, a racemate thereof, or any combination thereof, comprising a full agonist, a partial agonist, or an antagonist, or any combination thereof of a mu-opioid receptor is provided. A compound of Formula (I) or Formula (VI), its enantiomer, a racemate thereof, or any combination thereof, that also lacks agonist activity fully or partially against a kappa-opioid receptor, or a delta-opioid receptor, or both is provided. A compound of Formula (I), its enantiomer, a racemate thereof, or any combination thereof, comprising Compound 14, or Compound 22, or both is provided. A compound of Formula (I), its enantiomer, a racemate thereof, or any combination thereof, comprising Compound 19, Compound 26, or Compound 34, or any combination thereof is provided.

A pharmaceutical composition is provided that can comprise a therapeutically effective amount of a compound of Formula (I) or Formula (VI), its enantiomer, a racemate thereof, or any combination thereof, together with a pharmaceutically acceptable carrier. The pharmaceutically acceptable carrier can be, for example, selected from the group consisting of binders, buffering agents, coloring agents, diluents, disintegrants, emulsifiers, flavorants, glidants, lubricants, preservatives, stabilizers, surfactants, tableting agents, and wetting agents, and combinations thereof. The pharmaceutical composition can further comprise a therapeutically effective amount of an opioid. The opioid can be, for example, selected from alfentanil, buprenorphine, butorphanol, codeine, dezocine, dihydrocodeine, fentanyl, a fentanyl derivative, etonitazene, an etonitazene derivative, hydrocodone, hydromorphone, levorphanol, meperidine (pethidine), methadone, morphine, nalbuphine, oxycodone, oxymorphone, pentazocine, propiram, propoxyphene, sufentanil, tramadol, or a combination thereof. Any ratio of a compound of the present disclosure can be used in combination with such an opioid, or in combination with another compound of the present disclosure, or both, for example, in a ratio of 1:10,000, 1:1,000, 1:100, 1:10, 1:9, 1:7.5, 1:5, 1:3, 1:2.5, 1:2, 1:1.5, 1:1, or any opposite ratio, or any intervening ratio. The ratio can be varied over time, for example, as part of a opioid use disorder treatment or treatment of an opioid overdose. An opioid can be titrated down while a compound of the present disclosure is titrated up for patient administration.

A method of prevention or treatment of pain is provided that can comprise administering to a patient in need thereof a composition comprising a therapeutically effective amount of a compound of Formula (I) or Formula (VI), its enantiomer, a racemate thereof, or any combination thereof optionally in combination with one or more additional active ingredients. The one or more additional active ingredient can be, for example, an opioid selected from alfentanil, buprenorphine, butorphanol, codeine, dezocine, dihydrocodeine, fentanyl, a fentanyl derivative, etonitazene, an etonitazene derivative, hydrocodone, hydromorphone, levorphanol, meperidine (pethidine), methadone, morphine, nalbuphine, oxycodone, oxymorphone, pentazocine, propiram, propoxyphene, sufentanil, tramadol, or a combination thereof. The patient can be, for example, human. The pain can be associated, for example, with a gastrointestinal dysfunction. The gastrointestinal dysfunction can be, for example, irritable bowel syndrome, opioid-bowel dysfunction, colitis, post-operative and opioid-induced emesis, decreased gastric motility and emptying, inhibition of small and/or large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, abdominal or epigastric pain and discomfort, constipation, and delayed absorption of orally administered medications or nutritive substances. The pain can be associated with, for example, post-operative or opioid-induced ileus.

A method of treating or preventing an opioid use disorder (OUD) is provided that can comprise administering to a patient in need thereof a composition comprising a therapeutically effective amount of a compound of Formula (I) or Formula (VI), or any subformulae thereof, its enantiomer, a racemate thereof, or any combination thereof optionally in combination with one or more additional active ingredients. The method can comprise treating or preventing one or more symptoms associated with opioid withdrawal, comprising a therapeutically effective amount of a compound of Formula (I) or Formula (VI), or any subformulae thereof, its enantiomer, a racemate thereof, or any combination thereof optionally in combination with one or more additional active ingredients, for example, methadone, buprenorphine, naltrexone, clonidine, or lofexidine, or any combination thereof. A method of treating or preventing an opioid overdose is also provided that comprise administering to a patient in need thereof a composition comprising a therapeutically effective amount of a compound of Formula (I) or Formula (VI), or any subformulae thereof, its enantiomer, a racemate thereof, or any combination thereof optionally in combination with one or more additional active ingredients, for example, naloxone. The opioid overdose can comprise, for example, respiratory depression. The opioid overdose can comprise, for example, a fentanyl overdose, a fentanyl derivative overdose, an etonitazene overdose, or an etonitazene derivative overdose, or any combination thereof.

Reference to a formula includes references to all subformulae, for example, Formula (I) includes compounds of Formulae (Ia), (Ib), (II), (Ila), (IIb), (III), (IIIa), (IIIb), (IV), (IVa), (IVb), (V), (Va), (Vb), (XI), (XII), (XIII), (XIV), (XV), (XVI), (XVII), and (XVIII), as well as their enantiomers; and Formula (VI) includes compounds of Formulae (VIa), (VIb), (VII), (VIIa), (VIIb), (VIII), (VIIIa), (VIIIb), (IX), (IXa), (IXb), (X), (Xa), (Xb), and (XIX), as well as their enantiomers. Compounds disclosed herein can be administered as the neat chemical, or can be administered as a pharmaceutical composition. Accordingly, the disclosure encompasses pharmaceutical compositions comprising a therapeutically effective amount of a compound or pharmaceutically acceptable salt of a compound, together with at least one pharmaceutically acceptable carrier. The pharmaceutical composition can contain a therapeutically effective amount of the compound or salt as the only active agent, and can contain at least one additional active agent. The pharmaceutical composition can be in a dosage form that contains from about 0.1 mg to about 2000 mg, from about 10 mg to about 1000 mg, from about 100 mg to about 800 mg, or from about 200 mg to about 600 mg of a compound, and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition can also include a molar ratio of a compound and an additional active agent. For example, the pharmaceutical composition can contain a molar ratio of about 0.5:1, about 1:1, about 2:1, about 3:1 or from about 1.5:1 to about 4:1 of an additional active agent to a compound.

The pharmaceutical composition can further include a therapeutically effective amount of an opioid. The opioid can be selected from alfentanil, buprenorphine, butorphanol, codeine, dezocine, dihydrocodeine, fentanyl, a fentanyl derivative, etonitazene, an etonitazene derivative, hydrocodone, hydromorphone, levorphanol, meperidine (pethidine), methadone, morphine, nalbuphine, oxycodone, oxymorphone, pentazocine, propiram, propoxyphene, sufentanil, tramadol, or a combination thereof.

Compounds disclosed herein can be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition can be formulated as any pharmaceutically useful form, for example, as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, for example, a therapeutically effective amount to achieve the desired purpose.

Carriers include excipients and diluents of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers can be listed in more than one class, for example vegetable oil can be used as a lubricant in some formulations and a diluent in others. Exemplary pharmaceutically acceptable carriers include sugars, starches, celluloses, powdered tragacanth, malt, gelatin, talc, and vegetable oils. Optional active agents can be included in a pharmaceutical composition, which do not substantially interfere with the activity of the compound of the present disclosure.

The pharmaceutical compositions/combinations can be formulated for oral administration. These compositions contain between 0.1 and 99 weight % (wt %) of a compound and usually at least about 5 wt. % of a compound of Formula (I). Compositions can contain from about 25 wt % to about 50 wt % or from about 5 wt % to about 75 wt % of the compound.

Compounds disclosed herein, as well as pharmaceutical compositions comprising the compounds, are useful for treatment of pain. According to the present disclosure, a method of treating pain comprises providing to a patient in need of such treatment a therapeutically effective amount of a compound. The patient (subject) can be a mammal, and more specifically, a human, or non-human patients such as companion animals, for example, cats, dogs, and livestock animals.

A therapeutically effective amount of a pharmaceutical composition can be an amount sufficient to reduce or ameliorate the symptoms of a disease or condition. For example, a therapeutically effective amount can be an amount sufficient to reduce or ameliorate acute or chronic pain. A therapeutically effective amount of a compound or pharmaceutical composition described herein can also provide a sufficient concentration of a compound, when administered to a patient. A sufficient concentration can be a concentration of the compound in the patient's body to prevent or combat the feeling of pain. Such an amount can be ascertained experimentally, for example, by assaying blood concentration of the compound, or theoretically, by calculating bioavailability.

The methods of treatment disclosed herein include providing any suitable dosage amounts of a compound to a patient. Dosage levels of each compound of from about 0.1 mg to about 140 mg per kilogram of body weight per day are useful in the treatment of the above-indicated conditions (about 0.5 mg to about 7 g per patient per day). The amount of compound that can be combined with the carrier materials to produce a single dosage form will vary depending upon the patient treated and the particular mode of administration. Dosage unit forms can contain, for example, between from about 1 mg to about 500 mg of each active compound. For example, 25 mg to 500 mg, or 25 mg to 200 mg of a compound can be provided daily to a patient. Frequency of dosage can also vary depending on the compound used and the particular degree of pain. A dosage regimen of 4 times daily or less can be used, or a dosage regimen of 1 or 2 times daily can be used. The specific dose level for any particular patient can depend upon a variety of factors including the activity of the specific compound employed, the age, body weight, general health, sex, diet, time of administration, route of administration, and rate of excretion, drug combination, and the severity of the particular disease undergoing therapy.

A compound can be administered singularly (i.e., sole therapeutic agent of a regime) to treat or prevent pain, or can be administered in combination with another active agent. One or more compounds can be administered in coordination with a regime of one or more opioid-like analgesics. A method of treating pain in a mammal can include administering to the mammal a therapeutically effective amount of a compound, optionally, in combination with one or more additional active ingredients. The methods of treatment provided herein are also useful for treatment of mammals other than humans, including for veterinary applications such as to treat horses and livestock, for example, cattle, sheep, cows, goats, swine and the like, and pets (companion animals) such as dogs and cats.

For diagnostic or research applications, a wide variety of mammals will be suitable subjects including rodents (for example, mice, rats, hamsters), rabbits, primates, and swine such as inbred pigs and the like. Additionally, for in vitro applications, such as in vitro diagnostic and research applications, body fluids (for example, blood, plasma, serum, cellular interstitial fluid, saliva, feces, and urine) and cell and tissue samples of the above subjects will be suitable for use.

The present disclosure provides a method of treating pain or any other symptom or condition in a patient identified as in need of such treatment, the method comprising providing to the patient a therapeutically effective amount of a compound. The compounds provided herein can be administered alone, or in combination with one or more other active agents. The method of treating pain can additionally include administering the compound in combination with one or more additional compounds, wherein at least one of the additional compounds is an active agent, to a patient in need of such treatment. The one or more additional compounds can include additional therapeutic compounds, including an opioid selected from alfentanil, buprenorphine, butorphanol, codeine, dezocine, dihydrocodeine, fentanyl, a fentanyl derivative, etonitazene, an etonitazene derivative, hydrocodone, hydromorphone, levorphanol, meperidine (pethidine), methadone, morphine, nalbuphine, oxycodone, oxymorphone, pentazocine, propiram, propoxyphene, sufentanil, tramadol, or a combination thereof. Other opioids, optional conventional opioid components, and optional compounds for enhancing the analgesic potency of the opioid and/or for reducing analgesic tolerance development, can also be employed in the methods and compositions of the present disclosure.

The opioid component of the present compositions can further include one or more other active ingredients that can be conventionally employed in analgesic and/or cough-cold-antitussive combination products. Such conventional ingredients include, for example, aspirin, acetaminophen, phenylpropanolamine, phenylephrine, chlorpheniramine, caffeine, and/or guaifenesin. Typical or conventional ingredients that can be included in the opioid component are described, for example, in the Physicians' Desk Reference, 1999, the disclosure of which is hereby incorporated herein by reference, in its entirety.

The compounds employed in the methods of the present disclosure can exist in prodrug form. As used herein, “prodrug” is intended to include any covalently bonded carriers which release the active parent drug, for example, as according to a formula described herein, or other formulas or compounds employed in the methods of the present disclosure in vivo when such prodrug is administered to a mammalian subject. Because prodrugs are known to enhance numerous desirable qualities of pharmaceuticals (for example, solubility, bioavailability, manufacturing, etc.) the compounds employed in the present methods can, if desired, be delivered in prodrug form. Thus, the present disclosure contemplates methods of delivering prodrugs. Prodrugs of the compounds employed in the present disclosure can be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, iso-butyl, sec-butyl, tert-butyl, cyclopropyl, phenyl, benzyl, and phenethyl esters, and the like.

Opioid side effects, such as constipation, vomiting and nausea, can result from undesirable interaction of the opioid with peripheral opioid receptors, such as peripheral u receptors. Administration of the compounds of the present disclosure can block interaction of the opioid compounds with the peripheral receptors, thereby preventing and/or inhibiting the side effects, without interfering with the therapeutic effect of the opioid in the CNS. The compounds of the present disclosure can be used in methods to agonize μ, κ, or δ or any combinations or subcombinations of those opioid receptors, particularly where undesirable symptoms or conditions are side effects of administering exogenous opioids. The compounds can be used as to treat patients having disease states that are ameliorated by binding opioid receptors or in any treatment wherein temporary suppression of the μ, κ, or δ or any combination of opioid receptor system is desired.

Such symptoms, conditions or diseases can include, for example, the complete or partial agonism of opioid-induced sedation, confusion, respiratory depression, euphoria, dysphoria, hallucinations, pruritus (itching), increased biliary tone, increased biliary colic, and urinary retention, ileus, emesis, and addiction liability; prevention or treatment of opioid and cocaine dependence; rapid opioid detoxification; treatment of alcoholism; treatment of alcoholic coma; detection of opioid use or abuse (pupil test); treatment of eating disorders; treatment of obesity; treatment of post-concussional syndrome; adjunctive therapy in septic, hypovolemic or endotoxin-induced shock; potentiation of opioid analgesia (especially at ultra-low doses); reversal or prevention of opioid tolerance and physical dependence (especially at ultra-low doses); prevention of sudden infant death syndrome; treatment of psychosis (especially wherein the symptoms are associated with schizophrenia, schizophreniform disorder, schizoaffective disorder, unipolar disorder, bipolar disorder, psychotic depression, Alzheimer's disease, Parkinson's disease, compulsive disorders, and other psychiatric or neurologic disorders with psychosis as symptoms); treatment of dyskinesia, treatment of autism; treatment of the endocrine system (including increased release of leutinizing hormone, treatment of infertility, increasing number of multiple births in animal husbandry, and male and female sexual behavior); treatment of the immune system and cancers associated with binding of the opioid receptors; treatment of anxiolysis; treatment of diuresis; treatment and regulation of blood pressure; treatment of tinnitus or impaired hearing; treatment of epilepsy; treatment of cachexia; treatment of general cognitive dysfunctions; and treatment of kleptomania.

The compounds can also be used as cytostatic agents, as antimigraine agents, as immunomodulators, as immunosuppressives, as antiarthritic agents, as antiallergic agents, as virucides, to treat diarrhea, antipsychotics, as antischizophrenics, as antidepressants, as uropathic agents, as antitussives, as antiaddictive agents, as anti-smoking agents, to treat alcoholism, as hypotensive agents, to treat and/or prevent paralysis resulting from traumatic ischemia, general neuroprotection against ischemic trauma, as adjuncts to nerve growth factor treatment of hyperalgesia and nerve grafts, as anti-diuretics, as stimulants, as anti-convulsants, or to treat obesity. Additionally, the present compounds can be used in the treatment of Parkinson's disease as an adjunct to L-dopa for treatment dyskinesia associated with the L-dopa treatment.

The compounds can be used in methods for preventing or treating gastrointestinal dysfunction, including, but not limited to, irritable bowel syndrome, opioid-bowel dysfunction, colitis, post-operative and opioid-induced emesis (nausea and vomiting), decreased gastric motility and emptying, inhibition of small and/or large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, abdominal or epigastric pain and discomfort, constipation, and delayed absorption of orally administered medications or nutritive substances. The compounds can be used in methods for preventing or treating post-operative or opioid-induced ileus.

### EXAMPLES

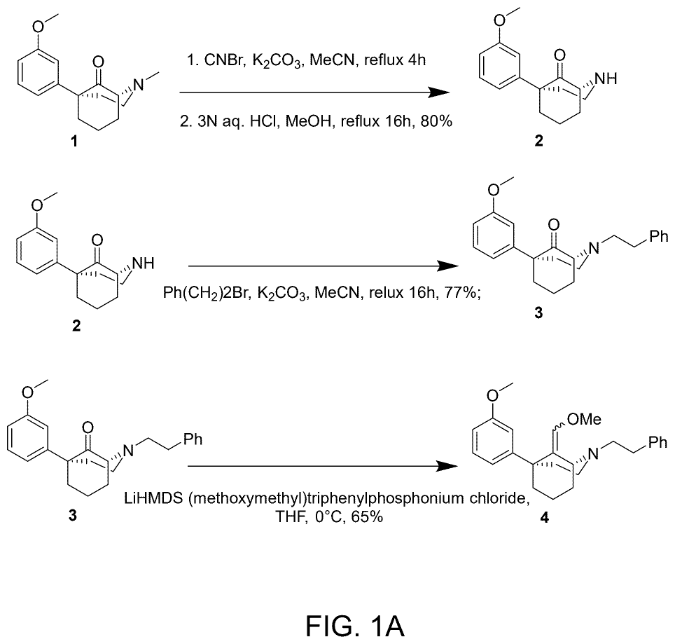

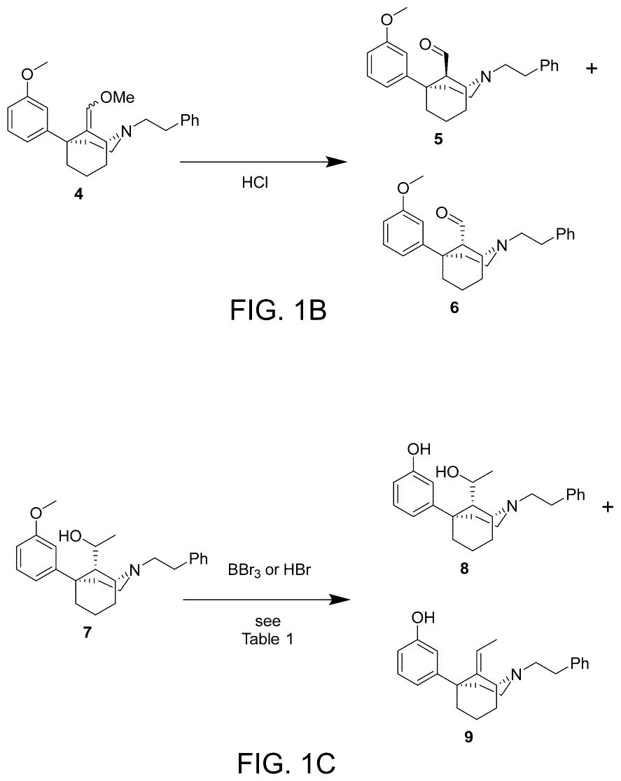

Synthetic pathways were development for access to various two-carbon C9-alkenyl 5-phenylmorphans using an intermediate, Compound 1 (Scheme 1; FIG. 1). This intermediate in a von Braun demethylation followed by alkylation with phenethyl bromide gave intermediate Compound 3 using standard procedures. The formation of enol ether Compound 4 was achieved by a Wittig olefination, which gave a 1:4 ratio of E/Z isomer. Hydrolysis of methyl vinyl ether Compound 4 with varying concentrations of HCl gave an epimeric mixture of aldehydes. As both epimers were of interest, both were reacted with 6N HCl. The aldehyde is chromatographically unstable; it was used without purification. A Grignard reaction was employed to introduce the alkyl chains. When methyl Grignard was added to the aldehyde epimeric mixture the expectation was to see a mixture of four diastereomeric products; however, only two major products were observed, one from each epimeric aldehyde, with a 10:1 selectivity. The C9-a and b isomers were separable by silica gel column chromatography. The less polar compound was the “b” isomer in this series of C9-alkenyl compounds and the “a” isomer was found to be the more polar compound. This was confirmed by X-ray diffraction analyses of various C9-substituted compounds. The O-demethylation product was obtained by treatment of Compound 7 with either BBr₃ or HBr (Table 1). When using HBr, short reaction times (<2 h) resulted in the desired product Compound 8 and extended reaction times resulted in unsaturated by-product Compound 9. Neither the O-demethylation product Compound 8 nor the ene product Compound 9 was observed when Compound 7 was treated with AlCl₃.

TABLE 1

| Reagent | Time | | CMP 8 | CMP 9 |
|---|---|---|---|---|
| BBr₃ | 17 | h | 86% | 0% |
| HBr | 2 | h | 50% | 0% |
| HBr | 17 | h | 0% | 42% |
| AlCl₃/PrSH | 2 | h | 0% | 0% |

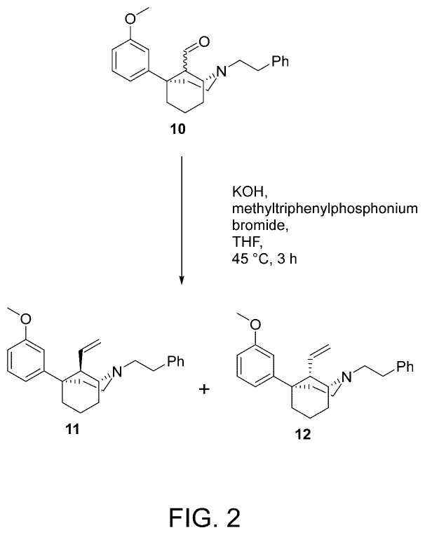

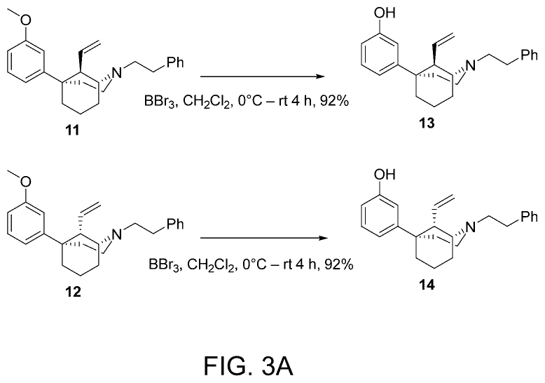

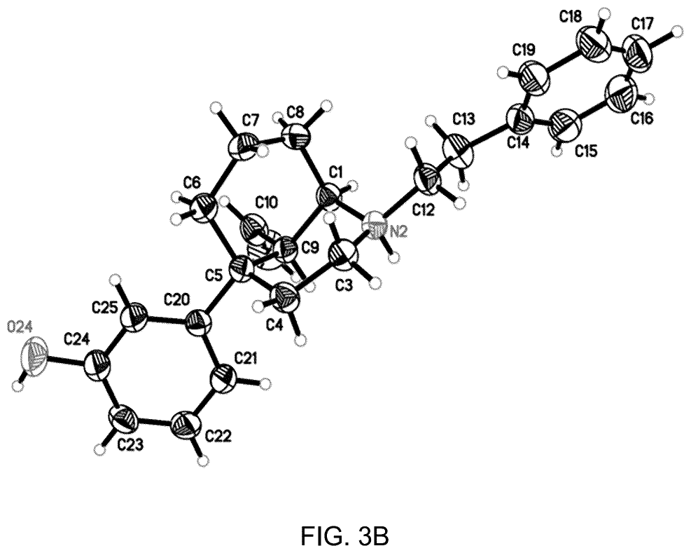

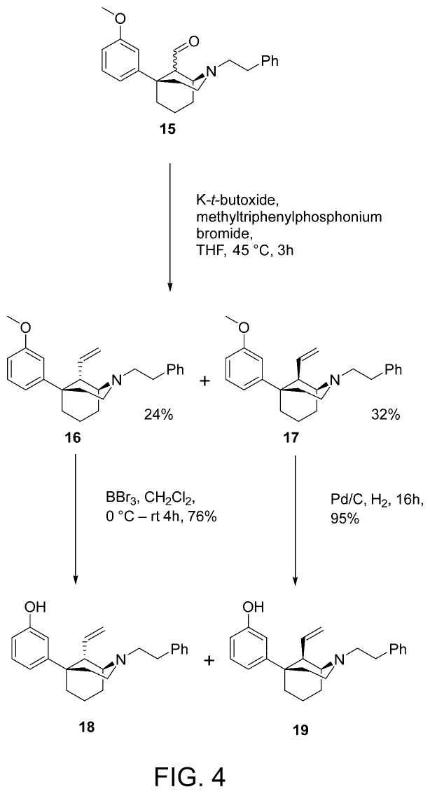

Alternatively, alkenyl derivatives were synthesized, from the common aldehyde intermediate Compound 10 using methyltriphenylphosphonium bromide in a Wittig reaction to yield Compound 11 and Compound 12 (Scheme 2; FIG. 2). The alkene products Compound 11 and Compound 12 were readily separated by silica gel flash chromatography and the C9-a isomer Compound 11 was subjected to O-demethylation conditions to form alkene Compound 13 (Scheme 3; FIG. 3). The absolute configuration of Compound 13 was confirmed by single crystal X-ray analysis (FIG. 3B). The same synthetic route starting from the C9 beta isomer Compound 12 (Scheme 4; FIG. 4) was employed to make the corresponding diasteriomers (Compounds 18 and 19).

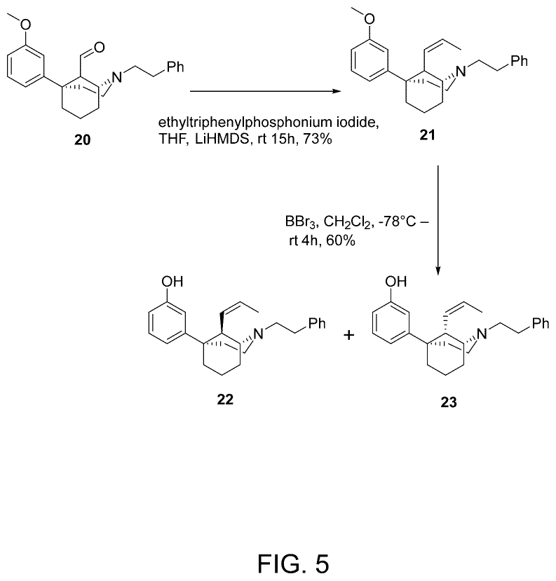

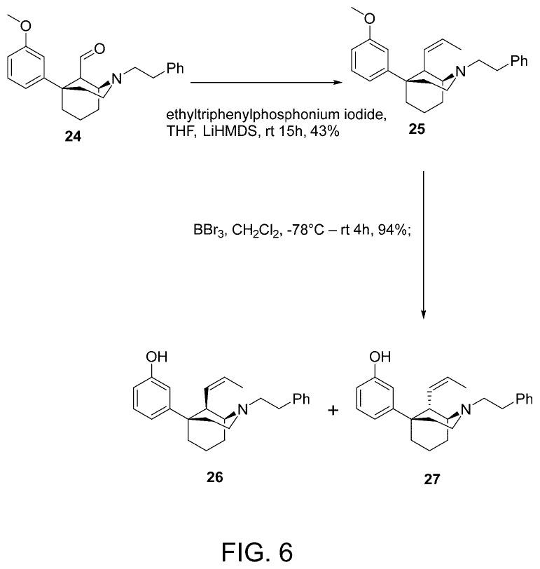

Synthesis of the C9-propenyl compounds was achieved similarly to the C9-ethenyl (vinyl) products. A Wittig reaction from aldehyde Compound 20 using LiHMDS and ethyltriphenylphosphonium iodide introduced the propylene moiety to the C-9 position (Scheme 5; FIG. 5). The Wittig products formed were an epimeric mixture Compound 21 that was not easily separable by column chromatography. The mixture was subjected to standard O-demethylation conditions to yield phenolic compounds Compound 22 and Compound 23 which were easily separable. The same synthetic steps were performed on the 1S,5R-phenylmorphans (Scheme 6; FIG. 6) to obtain the corresponding C9 propylene target compounds (Compound 26 and Compound 27).

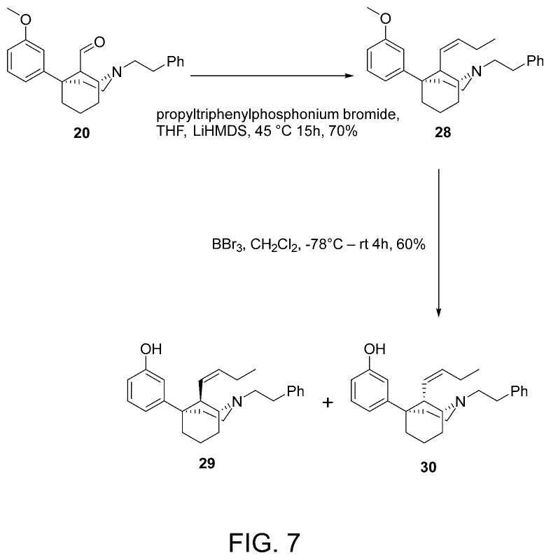

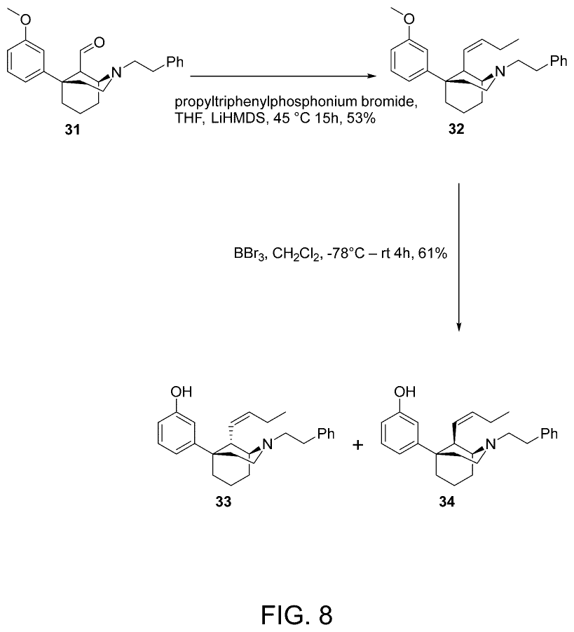

The C9-butylene compounds Compound 29 and Compound 30 were synthesized from the common aldehyde intermediate and propyltriphenylphosphinium bromide using LiHMDS as the base (Scheme 7; FIG. 7). This Wittig reaction involved heating at 45° C. for 15 h for consumption of starting material. The extended reaction times and heat resulted in more of the C9 alpha epimer to form from this reaction compared to the C9 beta epimer of Compound 28. This ratio was observed by 1H-NMR as these compounds were not easily separable by column chromatography. The mixture of epimers underwent O-demethylation using BBr₃ at which point they could be isolated. The same conditions were used to synthesize compounds Compound 33 and Compound 34.

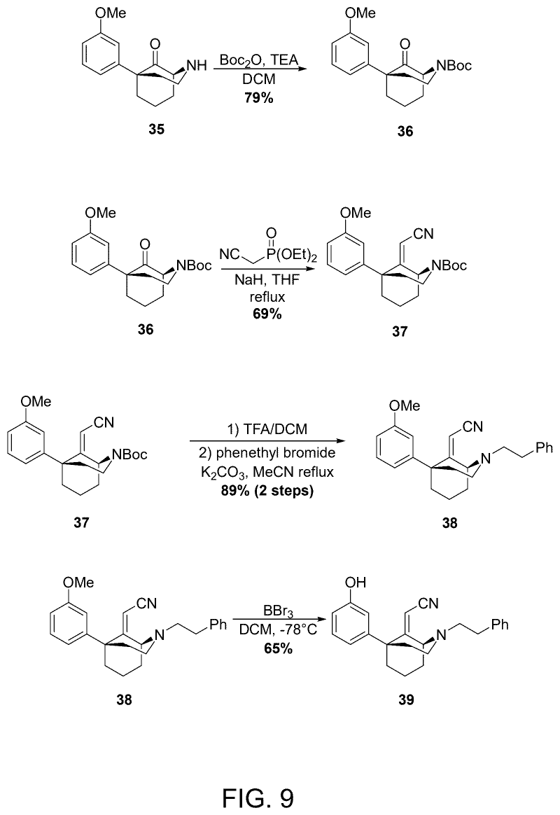

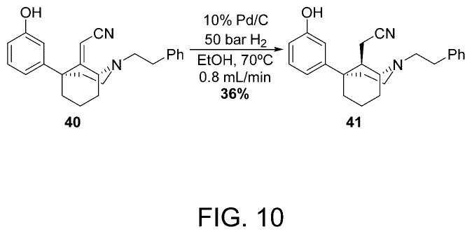

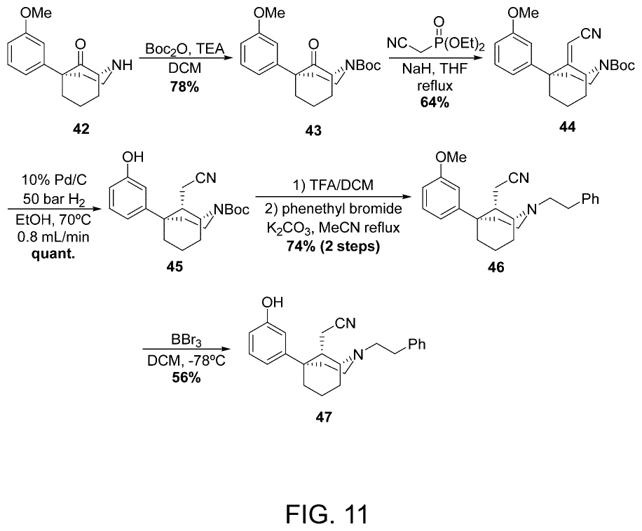

To accomplish the synthesis of the 9-cyano diastereomers, a Horner-Wadsworth-Emmons (HWE) reaction was performed on Boc-protected Compound 36, which gave an unsaturated nitrile that could be carried through Boc-removal, alkylation, and O-demethylation to access Compound 39 (Scheme 9; FIG. 9). There was a subsequent attempt to reduce the alkene in Compound 39 while leaving the nitrile intact. Employing traditional conditions with hydrogen and palladium on carbon as the catalyst resulted in a complex mixture of both alkene and partial nitrile reduction. A H-cube hydrogenation flow reactor was used to develop conditions that gave the desired product in moderate yield, favoring one diastereomer (Scheme 10; FIG. 10). To access the other diastereomer, reduction with the N-Boc analog was performed and the reaction gave the opposite stereochemistry at C9 (Scheme 11; FIG. 11).

Melting points were determined on a Mettler Toledo MP70 and were uncorrected. Proton and carbon nuclear magnetic resonance (1H and 13C NMR) spectra were recorded on a Varian Gemini-400 spectrometer in CDCl₃ (unless otherwise noted) with the values given in ppm (TMS as internal standard) and J (Hz) assignments of 1H resonance coupling. The analyses were performed on the free base, unless otherwise noted. Mass spectra (HRMS) were recorded on a VG 7070E spectrometer or a JEOL SX102a mass spectrometer. The optical rotation data were obtained on a PerkinElmer polarimeter model 341. Thin layer chromatography (TLC) analyses were carried out on Analtech silica gel GHLF 0.25 mm plates using various gradients of CHCl₃/MeOH containing 1% NH₄OH or gradients of EtOAc/n-hexane. Visualization was accomplished under UV light or by staining in an iodine chamber. Flash column chromatography was performed with Fluka silica gel 60 (mesh 220-400). Flash column chromatography was performed using RediSep Rf normal phase silica gel cartridges. Robertson Microlit Laboratories, Ledgewood, N.J., performed elemental analyses, and the results were within +0.4% of the theoretical values.

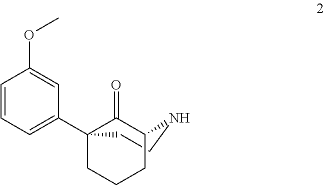 [SMILES: COc1cccc([C@@]23CCC[C@@H](NCC2)C3=O)c1]

(1S,5S)-5-(3-Methoxyphenyl)-2-azabicyclo[3.3.1]nonan-9-one (Compound 2): In an oven-dried flask, Compound 1 (2.2 g, 8.3 mmol) and K₂CO₃ (2.3 g, 16.6 mmol) in 15 mL acetonitrile were treated with 5M cyanogen bromide in acetonitrile (3.3 mL, 16.6 mmol). The reaction mixture was stirred under a N₂ atmosphere at room temperature for 2 h then heated to reflux. After 2 hours, the reaction mixture was extracted with CHCl₃, and the organic phase was washed with brine and concentrated. The residue was taken up in a mixture of 21 mL 3N HCl and 2.2 mL methanol and stirred at reflux for 17 hours. Upon completion, the reaction mixture was cooled and quenched with 7N NH₄OH in methanol. The mixture was extracted with CHCl₃ and washed with water, brine, dried with Na₂SO₄ and concentrated. Purification by flash column chromatography on silica gel (0-20% CMA in CHC (3) gave a red oil (1.62 g, 80% yield). The 1H NMR of the product was identical to that of standard.

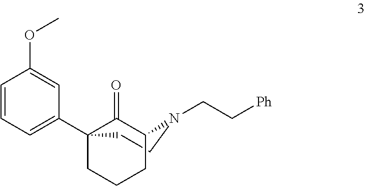 [SMILES: COc1cccc([C@]23CCC[C@H](C2=O)N(CCc2ccccc2)CC3)c1]

(1S,5S)-5-(3-Methoxyphenyl)-2-phenethyl-2-azabicyclo[3.3.1]nonan-9-one (Compound 3): In an oven-dried flask, Compound 2 (1.62 g, 6.6 mmol) and K₂CO₃ (1.82 g, 13.2 mmol) in 16 mL acetonitrile were treated with phenethyl bromide (1.34 mL, 9.9 mmol). The reaction mixture was stirred at reflux under a N₂ atmosphere. After 16 hours, the mixture was cooled, concentrated, and extracted with CHCl₃. Purification by flash column chromatography on silica gel (0-75% EtOAc in Hexanes) gave a brown oil (1.78 g, 77% yield). The 1H NMR of the product was identical to that of standard.

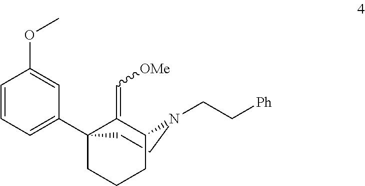 [SMILES: COC=C1[C@H]2CCC[C@]1(c1cccc(OC)c1)CCN2CCc1ccccc1]

(1S,5S)-9-(Methoxymethylene)-5-(3-methoxyphenyl)-2-phenethyl-2-azabicyclo[3.3.1]nonane (Compound 4): An oven-dried flask charged with Compound 3 (1.87 g, 5.1 mmol) and methoxy methylphosphonium chloride (5.25 g, 15.3 mmol) was evacuated and backfilled with N₂ three times. 11 mL of THF was added and the reaction mixture was cooled to 0° C. LHMDS (13.3 mL, 13.3 mmol) was added dropwise over 15 min. The reaction mixture was stirred at 0° C. for 30 minutes then allowed to warm to room temperature and stirred for an additional 22 hours. Upon completion, the reaction mixture was cooled to 0° C., quenched with methanol, concentrated, and extracted with CHCl₃, washed with water and brine, and dried with Na₂SO₄. Purification by flash column chromatography on silica gel (0-55% EtOAc in Hexanes) gave an orange oil (1.26 g, 65% yield, E:Z 1:4). The 1H NMR spectra of the product were identical to standard.

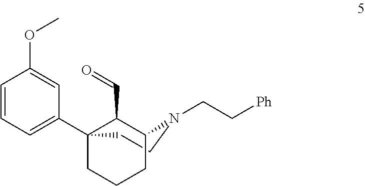 [SMILES: COc1cccc([C@]23CCC[C@H]([C@@H]2C=O)N(CCc2ccccc2)CC3)c1]

(RS)-1-((1S,5R,9R)-5-(3-Methoxyphenyl)-2-phenethyl-2-azabicyclo[3.3.1]nonan-9-yl) ethan-1-ol (Compound 5): A solution of Compound 4 (755 mg, 2 mmol) in 8 mL THF was treated with 16 mL 6N HCl and was stirred at room temperature. After 17 hours, the reaction mixture was quenched with NH₄OH, extracted with CHCl₃, washed with water and brine, dried with Na₂SO₄, and concentrated to obtain the aldehyde intermediate Compound 5 as an orange oil (C9 ratio β:α; 1.3:1). Aldehyde D Compound 5 was dried under high vacuum for 2 h then suspended in 8.2 mL Et₂O and cooled to 0° C. The reaction mixture was treated with methyl magnesium bromide dropwise over 5 minutes and stirred at 0° C. for 2 hours. Upon completion, the reaction was quenched with methanol and extracted with EtOAc. Purification by flash column chromatography on silica gel (0-100% EtOAc in hexanes) gave a green oil. (R_{f}=0.6 (EtOAc/hexanes). Crystallization from Et₂O gave a blue crystalline solid (307 mg, 40% yield), mp 103-105. ¹H NMR (400 MHZ; CDCl₃): δ 7.34-7.18 (m, 6H), 6.90-6.85 (m, 2H), 6.71 (dd, J=8.1, 2.2 Hz, 1H), 3.79 (s, 3H), 3.63 (q, J=6.3 Hz, 1H), 3.47 (d, J=2.1 Hz, 1H), 3.15 (dd, J=11.5, 3.6 Hz, 2H), 2.89-2.74 (m, 5H), 2.36-2.32 (m, 1H), 2.10-1.87 (m, 4H), 1.77-1.66 (m, 2H), 1.55-1.46 (m, 1H), 1.10 (d, J=6.3 Hz, 3H). 13C NMR (101 MHZ; CDCl₃): δ 159.5, 150.9, 139.8, 129.0, 128.58, 128.45, 126.2, 118.1, 112.5, 110.2, 67.1, 56.8, 55.1, 53.3, 48.70, 48.51, 42.5, 39.1, 34.5, 30.6, 25.6, 24.7, 22.9.

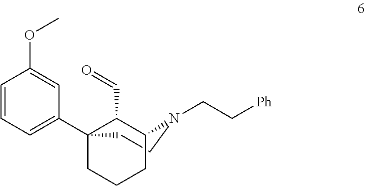 [SMILES: COc1cccc([C@]23CCC[C@H]([C@H]2C=O)N(CCc2ccccc2)CC3)c1]

(RS)-1-((1S,5R,9R)-5-(3-Methoxyphenyl)-2-phenethyl-2-azabicyclo[3.3.1]nonan-9-yl) ethan-1-ol (Compound 6): A solution of Compound 4 (755 mg, 2 mmol) in 8 mL THF was treated with 16 mL 6N HCl and was stirred at room temperature. After 17 hours, the reaction mixture was quenched with NH₄OH, extracted with CHCl₃, washed with water and brine, dried with Na₂SO₄, and concentrated to an orange oil (1.3:1 ratio β:α). Aldehyde Compound 6 was dried under high vacuum for 2 hours then suspended in 8.2 mL Et₂O and cooled to 0° C. The reaction mixture was treated with methyl magnesium bromide dropwise over 5 minutes and stirred at 0° C. for 2 hours. Upon completion, the reaction was quenched with methanol and extracted with EtOAc. Purification by flash column chromatography on silica gel (0-100% EtOAc in hexanes) gave an orange oil. R_{f}=0.3 (EtOAc/hexanes). Crystallization from Et₂O gave white crystalline solid (234 mg, 31% yield), mp 117-120. ¹H-NMR (400 MHZ; CDCl₃): δ 7.32-7.18 (m, 6H), 7.03 (d, J=7.9 Hz, 1H), 6.98 (s, 1H), 6.73 (d, J=8.2 Hz, 1H), 3.85-3.78 (m, 4H), 3.41 (s, 1H), 3.10-2.97 (m, 2H), 2.85 (qd, J=12.7, 6.9 Hz, 4H), 2.38-2.30 (m, 2H), 2.18-2.16 (m, 1H), 2.02-1.68 (m, 7H), 0.99-0.93 (m, 1H), 0.79 (d, J=6.3 Hz, 3H). 13C NMR (100 MHz; CDCl₃): δ 159.4, 151.7, 140.7, 128.85, 128.72, 128.3, 125.9, 118.8, 112.7, 110.5, 67.0, 58.1, 55.2, 52.9, 52.2, 49.9, 42.3, 38.4, 34.7, 29.9, 24.3, 21.8, 19.6.

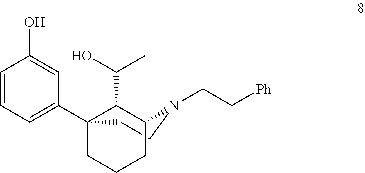 [SMILES: CC(O)[C@@H]1[C@H]2CCC[C@]1(c1cccc(O)c1)CCN2CCc1ccccc1]

3-((1S,5R,9R)-9-((R)-1-Hydroxyethyl)-2-phenethyl-2-azabicyclo[3.3.1]nonan-5-yl) phenol (Compound 8): In an oven-dried round-bottom flask, Compound 7 (120 mg, 0.32 mmol) was suspended in dichloromethane (4.5 mL) and the mixture was cooled to −78° C. Tribromoborane (120 μL, 3 equiv, 1.26 mmol) was added dropwise and the reaction was stirred at −78° C. The reaction mixture was allowed to warm to room temperature and stirred overnight (16 hours). Upon completion, the reaction mixture was cooled to 0° C. and quenched with MeOH and stirred for 30 minutes. 1N HCl (2 mL) was added and the reaction mixture was distilled at 100° C. for 1 hour. The reaction mixture was then cooled to 0° C. and made basic (>10.5) with NH₄OH and extracted with 9:1 CHCl3:MeOH. The combined organic layers were washed with water and brine, dried with sodium sulfate and concentrated. Purification by flash column chromatography on silica gel (20-100% EtOAc in hexanes) gave a yellow oil (100 mg, 86% yield). 1H NMR (400 MHZ; CDC (3): δ 7.31-7.25 (m, 2H), 7.24-7.09 (m, 4H), 6.82-6.74 (m, 2H), 6.64-6.61 (m, 1H), 3.70-3.64 (m, 1H), 3.58-3.56 (m, 1H), 3.24-3.17 (m, 2H), 2.99-2.78 (m, 5H), 2.38-2.31 (m, 1H), 2.07-1.83 (m, 5H), 1.76-1.71 (m, 3H), 1.09-1.04 (m, 3H). 13C NMR (100 MHz; CDC (3): δ 157.1, 150.2, 139.4, 129.3, 128.8, 128.7, 126.6, 117.0, 113.3, 112.8, 67.4, 56.6, 53.8, 48.7, 48.6, 42.2, 38.9, 34.1, 30.5, 25.6, 24.6, 22.8 [a]²⁰_{D} 21.9° (c 0.54, MeOH) HRMS-ESI (m/z): [M+H⁺] calcd for: C₂₄H₃₂NO₂ 366.2427; found 366.2433.

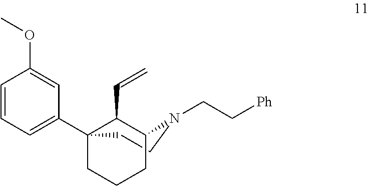 [SMILES: C=C[C@H]1[C@H]2CCC[C@]1(c1cccc(OC)c1)CCN2CCc1ccccc1]

(1R,5S,9R)-5-(3-Methoxyphenyl)-2-phenethyl-9-vinyl-2-azabicyclo[3.3.1]nonane (Compound 11): In an oven-dried flask, Compound 4 (650 mg, 1.56 mmol) was suspended in THF (7 mL) and treated with HCl (6 M, 12 mL) and the reaction mixture was stirred overnight at room temperature under argon. The reaction mixture was quenched with 7N NH₄OH in MeOH, extracted with CHCl₃ and washed with water and brine. The organic layer was then dried with sodium sulfate, concentrated, and put under high vacuum for 2 hours. An oven-dried round-bottom flask flushed with argon was charged with methyltriphenylphosphonium bromide (1.67 g, 3 equiv, 4.69 mmol) and potassium 2-methylpropan-2-olate (526 mg, 3 equiv, 4.69 mmol) and suspended in THF (15 mL). The reaction mixture was heated to 45° C. for 1 hour and turned yellow when the ylide was formed. The dried aldehyde was suspended in THF (10 mL) and was transferred to the ylide mixture. The reaction mixture was stirred at 45° C. for 2 hours when it was complete by TLC, whereupon the reaction mixture was quenched with NH₄OH in MeOH and extracted with EtOAc. The mixture was washed with water and brine, dried with sodium sulfate and concentrated. Purification by flash column chromatography on silica gel (0-100% EtOAc in hexanes) yielded a green oil (233 mg, 37% yield). Crystallization as the oxalate salt from acetone afforded a white solid: mp 170-175° C.; 1H-NMR (400 MHZ; CD₃OD): δ 7.33 (s, 4H), 7.33-7.20 (m, 3H), 6.97-6.91 (m, 2H), 6.76 (dd, J=8.1, 1.2 Hz, 1H), 5.76-5.67 (m, 1H), 5.14 (m, 2H), 3.80-3.74 (m, 4H), 3.68-3.61 (m, 2H), 3.56-3.44 (m, 2H), 3.38 (dd, J=5.5, 0.6 Hz, 1H), 3.14-3.11 (m, 2H), 2.41-1.93 (m, 8H). 13C NMR (101 MHZ; CD₃OD): δ 164.9, 159.8, 148.2, 136.5, 134.4, 129.0, 128.5, 126.8, 118.6, 117.8, 112.0, 110.9, 60.7, 55.7, 54.2, 49.4, 46.5, 37.8, 36.7, 30.4, 28.0, 19.6, 16.8; HRMS-ESI (m/z): [M+H⁺] calcd for: C₂₅H₃₂NO 362.2484; found: 362; [a]²⁰_{D}, 18.2° (c 0.74, CHCl₃).

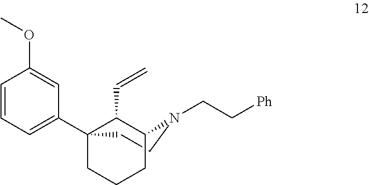 [SMILES: C=C[C@@H]1[C@H]2CCC[C@]1(c1cccc(OC)c1)CCN2CCc1ccccc1]

(1R,5S,9S)-5-(3-Methoxyphenyl)-2-phenethyl-9-vinyl-2-azabicyclo[3.3.1]nonane (Compound 12): Enol ether Compound 4 was subjected to the same reaction conditions as the diastereomer. Isolated as a yellow oil (160 mg, 26% yield) Crystallization as the oxalate salt from acetone afforded a white solid: mp 180-184° C.; 1H NMR (400 MHZ; CD₃OD): δ 7.35-7.20 (m, 6H), 6.89-6.84 (m, 2H), 6.76 (dd, J=8.2, 2.2 Hz, 1H), 5.73-5.64 (m, 1H), 5.35-5.24 (m, 2H), 3.94 (d, J=8.6 Hz, 1H), 3.78-3.74 (m, 3H), 3.74-3.67 (m, 1H), 3.59-3.50 (m, 2H), 3.41-3.31 (m, 2H), 3.17-3.09 (m, 1H), 2.96-2.88 (m, 1H), 2.52-2.37 (m, 3H), 2.18-1.81 (m, 5H); 13C NMR (101 MHz; CD₃OD): δ 166.2, 161.3, 149.7, 137.4, 136.3, 130.5, 130.05, 129.88, 128.4, 120.9, 119.0, 113.4, 112.2, 60.8, 56.6, 55.7, 51.7, 47.4, 41.8, 38.1, 31.5, 29.3, 24.1, 22.0; HRMS-ESI (m/z): [M+H⁺] calcd for: C₂₅H₃₂NO 362.2484; found: 362.2485; [a]²⁰_{D} −49.1° (c 0.2, CHCl₃).

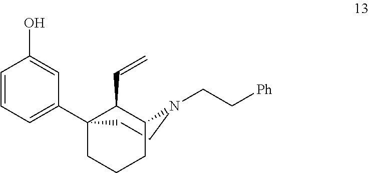 [SMILES: C=C[C@H]1[C@H]2CCC[C@]1(c1cccc(O)c1)CCN2CCc1ccccc1]

3-((1R,5S,9R)-2-Phenethyl-9-vinyl-2-azabicyclo[3.3.1]nonan-5-yl) phenol (Compound 13): In an oven-dried round-bottom flask, Compound 11 (280 mg, 1 equiv, 360 μmol) was suspended in dichloromethane (6 mL) and the mixture was cooled to −78° C. Tribromoborane (147 μL, 3 equiv, 1.44 mmol) was added dropwise and the reaction was stirred at −78° C. The reaction mixture was allowed to warm to room temperature and stirred overnight (16 hours). Upon completion, the reaction mixture was cooled to 0° C. and quenched with MeOH and stirred for 30 minutes. 1N HCl (2 mL) was added, and the reaction mixture was distilled at 100° C. for 1 hour. The reaction mixture was then cooled to 0° C. and made basic (>10.5) with NH₄OH and extracted with 9:1 CHCl3:MeOH. The combined organic layers were washed with water and brine, dried with sodium sulfate and concentrated. Purification by flash column chromatography on silica gel (20-100% EtOAc in hexanes) gave a yellow oil (248 mg, 92% yield). The HCl salt of Compound 13 was formed in iPrOH (1 mL) with 37% HCl (0.1 mL) and recrystallized from hot ethanol (3 mL) to give a white solid: mp 233-237° C.; 1H NMR (400 MHz; CD₃OD): δ 7.38-7.33 (m, 4H), 7.28 (dq, J=8.8, 4.3 Hz, 1H), 7.14 (t, J=8.0 Hz, 1H), 6.88-6.83 (m, 2H), 6.63 (dd, J=8.0, 1.9 Hz, 1H), 5.73 (tt, J=10.4, 7.0 Hz, 1H), 5.22-5.14 (m, 2H), 3.78 (d, J=0.2 Hz, 1H), 3.72-3.60 (m, 2H), 3.51 (dd, J=10.0, 6.8 Hz, 2H), 3.39 (dd, J=5.5, 0.4 Hz, 1H), 3.16 (dd, J=10.4, 6.6 Hz, 2H), 2.38-1.93 (m, 8H); 13C NMR (101 MHZ; CD₃OD): δ 158.6, 149.4, 137.7, 135.8, 130.4, 129.99, 129.89, 128.3, 120.0, 118.0, 114.3, 114.1, 62.0, 57.0, 51.1, 48.0, 39.4, 38.0, 31.8, 29.4, 21.1, 18.1; HRMS-ESI (m/z): [M+H⁺] calcd for: C₂₄H₃₀NO 348.2327; found: 348.2328; Anal. Calcd. For C₂₄H₃₀ClNO:C, 75.08%; H, 7.88%; N, 3.65%. Found C, 75.12%; H 7.84%; N, 3.64%; [a]_{D}²⁰, 22.5° (c 0.64, CHCl₃).

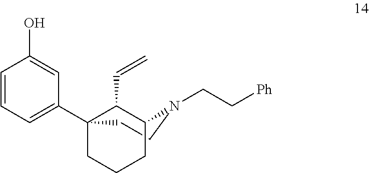 [SMILES: C=C[C@@H]1[C@H]2CCC[C@]1(c1cccc(O)c1)CCN2CCc1ccccc1]

3-((1R,5S,9S)-2-Phenethyl-9-vinyl-2-azabicyclo[3.3.1]nonan-5-yl) phenol (Compound 14): In an oven-dried round-bottom flask, Compound 12 (280 mg, 1 equiv, 360 μmol) was suspended in dichloromethane (6 mL) and the mixture was cooled to −78° C. Tribromoborane (147 μL, 3 equiv, 1.44 mmol) was added dropwise and the reaction was stirred at −78° C. The reaction mixture was allowed to warm to room temperature and stirred overnight (16 h). Upon completion, the reaction mixture was cooled to 0° C. and quenched with MeOH and stirred for 30 min. 1N HCl (2 mL) was added, and the reaction mixture was distilled at 100° C. for 1 hour. The reaction mixture was then cooled to 0° C. and made basic (>10.5) with NH₄OH and extracted with 9:1 CHCl₃:MeOH. The combined organic layers were washed with water and brine, dried with sodium sulfate and concentrated. Purification by flash column chromatography on silica gel (20-100% EtOAc in hexanes) gave a yellow oil (248 mg, 92% yield). The HCl salt of Compound 14 was formed in iPrOH (0.5 mL) with 37% HCl (0.05 mL) and recrystallized from hot ethanol (3 mL) to give a white solid (173 mg, 64% yield). The HCl salt of Compound 14 was formed in iPrOH with 37% HCl (0.1 mL) and recrystallized from ethanol to give a white solid: mp 264-267° C.; 1H NMR (400 MHZ; CD3OD): δ 7.38-7.26 (m, 5H), 7.13 (t, J=8.0 Hz, 1H), 6.79 (d, J=7.9 Hz, 1H), 6.75 (s, 1H), 6.63 (dd, J=8.0, 1.7 Hz, 1H), 5.74 (ddd, J=17.4, 10.7, 6.7 Hz, 1H), 5.33 (dd, J=24.4, 14.0 Hz, 2H), 3.97 (d, J=0.5 Hz, 1H), 3.77-3.69 (m, 1H), 3.57 (td, J=11.9, 5.9 Hz, 2H), 3.38 (td, J=12.1, 5.2 Hz, 1H), 3.31 (t, J=1.5 Hz, 2H), 3.16 (td, J=12.2, 5.6 Hz, 1H), 2.98-2.90 (m, 1H), 2.51-2.36 (m, 3H), 2.20-2.03 (m, 2H), 2.01-1.83 (m, 3H); 13C NMR (101 MHz; CD3OD): δ 158.6, 149.7, 137.4, 136.4, 130.5, 130.04, 129.9, 128.4, 120.9, 117.8, 114.2, 113.8, 60.5, 56.4, 51.9, 47.3, 41.8, 38.0, 31.5, 29.3, 24.0, 22.0. HRMS-ESI (m/z): [M+H⁺] calcd for: C₂₄H₃₀NO 348.2327; found: 348.2328; Anal. Calcd. For C₂₄H₃₀ClNO·0.5 H2O: C, 73.36%; H, 7.95%; N, 3.56%. Found C, 73.11%; H, 7.68%; N, 3.55%; [a]20D−35.8° (c 0.45, CHCl₃).

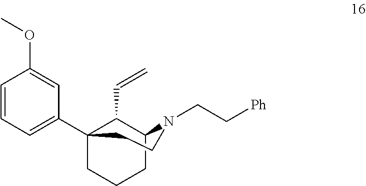 [SMILES: C=C[C@@H]1[C@@H]2CCC[C@@]1(c1cccc(OC)c1)CCN2CCc1ccccc1]

(1S,5R,9S)-5-(3-Methoxyphenyl)-2-phenethyl-9-vinyl-2-azabicyclo[3.3.1]nonane (Compound 16): In an oven-dried flask, Compound 4 (590 mg, 1.56 mmol) was suspended in THF (6 mL) and treated with HCl (6 M, 10 mL) and the reaction mixture was stirred overnight at room temperature under argon. The reaction mixture was quenched with 7N NH₄OH in MeOH, extracted with CHCl₃ and washed with water and brine. The organic layer was then dried with sodium sulfate, concentrated, and put under high vacuum for 2 hours. An oven-dried round-bottom flask flushed with argon was charged with methyltriphenylphosphonium bromide (1.67 g, 3 equiv, 4.69 mmol) and potassium 2-methylpropan-2-olate (526 mg, 3 equiv, 4.69 mmol) and suspended in THF (13 mL). The reaction mixture was heated to 45° C. for 1 hour and turned yellow when the ylide was formed. The dried aldehyde was suspended in THF (10 mL) and was transferred to the ylide mixture. The reaction mixture was stirred at 45° C. for 2 h when it was complete by TLC, whereupon the reaction mixture was quenched with NH₄OH in MeOH and extracted with EtOAc. The mixture was washed with water and brine, dried with sodium sulfate and concentrated. Purification by flash column chromatography on silica gel (0-100% EtOAc in hexanes) gave a green oil (183 mg, 32% yield). δ 7.32-7.17 (m, 6H), 6.99-6.94 (m, 2H), 6.72-6.69 (m, 1H), 5.76 (ddd, J=17.4, 10.4, 7.8 Hz, 1H), 5.09-4.98 (m, 2H), 3.81-3.76 (m, 3H), 3.08 (td, J=13.8, 4.3 Hz, 4H), 2.91-2.78 (m, 4H), 2.21-1.77 (m, 7H), 1.60 (ddt, J=17.1, 9.6, 4.4 Hz, 1H). 13C NMR (100 MHz; CDCl₃): δ 159.3, 151.4, 140.5, 138.3, 128.9, 128.7, 128.4, 126.0, 118.4, 116.9, 112.4, 110.4, 58.4, 57.9, 55.1, 49.8, 49.4, 41.22, 38.1, 34.6, 29.7, 22.1, 19.3.

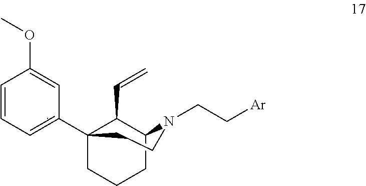

(1S,5R,9R)-5-(3-Methoxyphenyl)-2-phenethyl-9-vinyl-2-azabicyclo[3.3.1]nonane (Compound 17): Enol ether Compound 4 (590 mg, 1.56 mmol) was suspended in THF (6 mL) and treated with HCl (6 M, 10 mL) and the reaction mixture was stirred overnight at room temperature under argon. The reaction mixture was quenched with 7N NH₃OH in MeOH, extracted with CHCl₃ and washed with water and brine. The organic layer was then dried with sodium sulfate and concentrated and dried under reduced pressure for 2 hours. An oven-dried round-bottom flask flushed with argon was charged with methyltriphenylphosphonium bromide (1.67 g, 3 equiv, 4.69 mmol) and potassium 2-methylpropan-2-olate (526 mg, 3 equiv, 4.69 mmol). THF (13 mL) was added, and the reaction mixture was heated to 45° C. for 1 hour. The solution turned yellow when the ylide was formed and the aldehyde mixture was added and stirred at 45° C. for 2 hours. Upon completion by TLC, the reaction mixture was quenched with NH₄OH in MeOH and extracted with EtOAc. The mixture was washed with water and brine, dried with sodium sulfate and concentrated. Purification by flash column chromatography on silica gel (0-100% EtOAc in hexanes) gave a blue oil (138 mg, 24% yield). 1H NMR (400 MHZ; CDC (3): δ 7.36-7.32 (m, 2H), 7.27-7.17 (m, 4H), 6.94-6.89 (m, 2H), 6.71 (dd, J=8.1, 2.0 Hz, 1H), 6.01 (dt, J=17.4, 8.9 Hz, 1H), 4.94 (t, J=15.2 Hz, 2H), 3.84-3.79 (m, 3H), 3.16 (dq, J=18.1, 5.9 Hz, 3H), 2.86-2.71 (m, 5H), 2.46-2.31 (m, 2H), 2.10-1.67 (m, 5H), 1.60-1.49 (m, 1H).

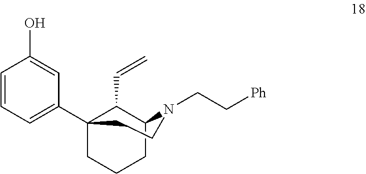 [SMILES: C=C[C@@H]1[C@@H]2CCC[C@@]1(c1cccc(O)c1)CCN2CCc1ccccc1]

3-((1S,5R,9S)-2-Phenethyl-9-vinyl-2-azabicyclo[3.3.1]nonan-5-yl) phenol (Compound 18): In an oven-dried round-bottom flask, Compound 16 (750 mg, 1 equiv, 360 μmol) was suspended in dichloromethane (3 mL) and the mixture was cooled to −78° C. Tribromoborane (136 μL, 3 equiv, 1.44 mmol) was added dropwise and the reaction was stirred at −78° C. The reaction mixture was allowed to warm to room temperature and stirred overnight (16 hours). Upon completion, the reaction mixture was cooled to 0° C. and quenched with MeOH and stirred for 30 minutes. 1N HCl (2 mL) was added, and the reaction mixture was distilled at 100° C. for 1 hour. The reaction mixture was then cooled to 0° C. and made basic (>10.5) with NH₄OH and extracted with 9:1 CHCl₃:MeOH. The combined organic layers were washed with water and brine, dried with sodium sulfate and concentrated. Purification by flash column chromatography on silica gel (20-100% EtOAc in hexanes) gave a yellow oil (558 mg, 77% yield). The HCl salt of Compound 18 was formed in iPrOH (2 mL) with 37% HCl (0.2 mL) and recrystallized from hot ethanol (4 mL) and cooled, stirring 16 hours to give a white solid: mp 231-235° C.; 1H-NMR (400 MHZ; CD3OD): δ 7.36 (d, J=13.0 Hz, 4H), 7.28 (dq, J=8.5, 4.3 Hz, 1H), 7.13 (t, J=8.0 Hz, 1H), 6.88-6.84 (m, 2H), 6.63 (dd, J=8.0, 2.0 Hz, 1H), 5.77-5.68 (m, 1H), 5.22-5.13 (m, 2H), 3.78 (s, 1H), 3.71-3.58 (m, 2H), 3.51 (quintet, J=8.9 Hz, 2H), 3.41 (d, J=5.6 Hz, 1H), 3.17 (dd, J=10.4, 6.5 Hz, 2H), 2.39-1.92 (m, 8H). 13C NMR (101 MHz; CD3OD): δ 158.6, 149.5, 137.8, 135.8, 130.4, 129.98, 129.91, 128.3, 120.0, 118.0, 114.3, 114.1, 62.0, 57.0, 51.0, 47.9, 39.3, 38.0, 31.8, 29.4, 21.1, 18.1. HRMS-ESI (m/z): [M+H⁺] calcd for: C₂₅H₃₂NO 348.2327; found: 348.2328; Anal. Calcd For C₂₄H₃₀ClNO:C, 75.08%; H, 7.88%; N, 3.65%. Found C, 75.00%; H, 7.86%; N, 3.64%; [a]20D −24.3° (c 0.64, CHCl₃).

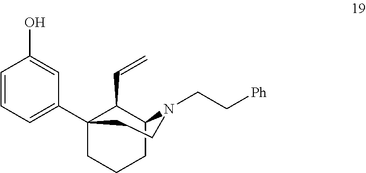 [SMILES: C=C[C@H]1[C@@H]2CCC[C@@]1(c1cccc(O)c1)CCN2CCc1ccccc1]

3-((1S,5R,9R)-2-Phenethyl-9-vinyl-2-azabicyclo[3.3.1]nonan-5-yl) phenol (Compound 19): In an oven-dried round-bottom flask, Compound 17 (750 mg, 1 equiv, 360 μmol) was suspended in dichloromethane (3 mL) and the mixture was cooled to −78° C. Tribromoborane (136 μL, 3 equiv, 1.44 mmol) was added dropwise and the reaction was stirred at −78° C. The reaction mixture was allowed to warm to room temperature and stirred overnight (16 hours). Upon completion, the reaction mixture was cooled to 0° C. and quenched with MeOH and stirred for 30 minutes. 1N HCl (2 mL) was added and the reaction mixture was distilled at 100° C. for 1 hour. The reaction mixture was then cooled to 0° C. and made basic (>10.5) with NH₄OH and extracted with 9:1 CHCl3:MeOH. The combined organic layers were washed with water and brine, dried with sodium sulfate and concentrated. Purification by flash column chromatography on silica gel (20-100% EtOAc in hexanes) gave a yellow oil (554 mg, 76% yield). The HCl salt of Compound 19 was formed in iPrOH with 37% HCl (0.1 mL) and recrystallized from ethanol to give a white solid: mp 264-266° C.; 1H-NMR (400 MHz; CD3OD): δ 7.38-7.26 (m, 5H), 7.13 (t, J=8.0 Hz, 1H), 6.79 (d, J=7.9 Hz, 1H), 6.75 (t, J=1.9 Hz, 1H), 6.63 (dd, J=8.0, 2.3 Hz, 1H), 5.74 (ddd, J=17.4, 10.7, 6.7 Hz, 1H), 5.33 (dd, J=25.2, 14.0 Hz, 2H), 3.97 (t, J=0.5 Hz, 1H), 3.73 (td, J=13.3, 5.9 Hz, 1H), 3.61-3.54 (m, 2H), 3.38 (td, J=12.1, 5.2 Hz, 1H), 3.16 (td, J=12.2, 5.6 Hz, 1H), 2.94 (td, J=12.1, 5.1 Hz, 1H), 2.51-2.36 (m, 3H), 2.20-1.83 (m, 5H); 13C NMR (101 MHz; CD3OD): δ 158.6, 149.7, 137.4, 136.4, 130.5, 130.04, 129.9, 128.4, 120.9, 117.8, 114.2, 113.8, 60.5, 56.4, 51.9, 47.3, 41.8, 38.0, 31.5, 29.3, 24.0, 22.0; HRMS-ESI (m/z): [M+H+] calcd for: C₂₄H₃₀NO 348.2327; found: 348.2328; Anal. Calcd. For C24H30ClNO:C, 75.08%; H, 7.88%; N, 3.65%. Found C, 74.78%; H, 7.48%; N, 3.61%; [a]20D −35.6° (c 0.95, CHCl₃).

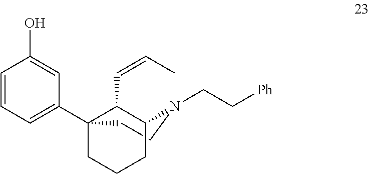 [SMILES: C/C=C\[C@@H]1[C@H]2CCC[C@]1(c1cccc(O)c1)CCN2CCc1ccccc1]

3-((1R,5S,9S)-2-Phenethyl-9-((E)-prop-1-en-1-yl)-2-azabicyclo[3.3.1]nonan-5-yl) phenol (Compound 23): In an oven-dried round-bottom flask, Compound 21 (250 mg, 1 equiv, 1.33 mmol) was suspended in dichloromethane (15 mL) and the mixture was cooled to −78° C. Tribromoborane (667 mg, 253 μL, 2 equiv, 2.66 mmol) was added to drop-wise and the reaction was stirred at −78° C. for 15 minutes. The reaction mixture was allowed to warm to room temperature and stirred 2 h. Upon completion, the reaction mixture was cooled to 0° C. and quenched with 7 mL MeOH dropwise and stirred for 30 min. subsequently, 10 mL 1N HCl was added, and the reaction mixture was distilled at 100° C. for 1 hour. The reaction mixture was then cooled to 0° C. and made basic (>10.5) with NH₄OH and extracted with 9:1 CHCl3:MeOH. The combined organic layers were washed with water and brine, dried with sodium sulfate and concentrated. Purification by silica gel column chromatography 0-60% EtOAc:Hexanes. Compound 23 was isolated as a white foam (133 mg, 55% yield) as the less polar fraction. The HCl salt of Compound 23 was formed in iPrOH with 37% HCl (0.1 mL) and recrystallized from ethanol to give a white solid: mp 260-264° C. 1H NMR (400 MHZ; CD3OD): δ 7.35-7.24 (m, 5H), 7.08 (t, J=8.0 Hz, 1H), 6.73 (d, J=8.0 Hz, 1H), 6.68 (d, J=1.8 Hz, 1H), 6.59 (dd, J=8.0, 1.8 Hz, 1H), 5.70-5.63 (m, 1H), 5.34-5.29 (m, 1H), 3.73-3.67 (m, 2H), 3.59-3.46 (m, 3H), 3.39-3.32 (m, 1H), 3.15-3.07 (m, 1H), 2.93-2.86 (m, 1H), 2.55-2.46 (m, 1H), 2.40-2.35 (m, 2H), 2.17-2.03 (m, 2H), 1.99-1.82 (m, 3H), 1.76 (dd, J=7.0, 1.3 Hz, 3H). 13C NMR (100 MHz; CD3OD): δ 158.5, 150.0, 137.4, 131.0, 130.3, 130.03, 129.88, 128.4, 127.7, 117.5, 114.1, 113.6, 60.4, 56.5, 51.8, 42.35, 42.19, 37.8, 31.5, 29.1, 23.9, 22.1, 13.7; HRMS-ESI (m/z): [M+H+] calcd for: C₂₅H₃₂NO 362.2484; found: 362.2486; C₂₅H₃₂ClNO·1.45 H2O calc. C: 70.8; H: 8.29; N: 3.3; found C: 70.5; H: 7.92; N: 3.3; [a]20D −22.4° (c 1.0, CHCl₃).

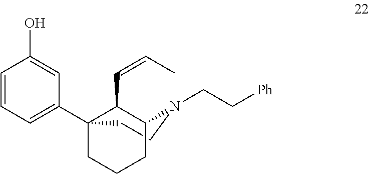 [SMILES: C/C=C\[C@H]1[C@H]2CCC[C@]1(c1cccc(O)c1)CCN2CCc1ccccc1]

3-((1R,5S,9R)-2-phenethyl-9-((E)-prop-1-en-1-yl)-2-azabicyclo[3.3.1]nonan-5-yl) phenol (Compound 22): From the same procedure as Compound 23, Compound 22 was isolated as a white foam (153, 32% yield) as the more polar fraction. The HCl salt of Compound 22 was formed in iPrOH with 37% HCl (0.1 mL) and recrystallized from ethanol to give a white solid: mp 271-275° C.; 1H NMR (400 MHZ; CD3OD): δ 7.38-7.33 (m, 4H), 7.31-7.26 (m, 1H), 7.11 (t, J=8.0 Hz, 1H), 6.84-6.79 (m, 2H), 6.61 (dd, J=8.0, 2.2 Hz, 1H), 5.61-5.53 (m, 1H), 5.39 (ddd, J=10.6, 8.8, 1.7 Hz, 1H), 3.68 (dt, J=21.3, 7.7 Hz, 4H), 3.52-3.48 (m, 2H), 3.15 (dt, J=10.3, 6.0 Hz, 2H), 2.34-2.19 (m, 3H), 2.19-1.91 (m, 5H), 1.76 (dd, J=6.9, 1.6 Hz, 3H). 13C NMR (100 MHz; CD₃OD): δ 158.5, 149.8, 137.7, 130.3, 130.00, 129.91, 129.5, 128.3, 127.4, 117.7, 114.2, 113.8, 61.1, 56.8, 51.1, 41.9, 39.9, 37.8, 31.8, 29.2, 21.0, 18.7, 13.8; HRMS-ESI (m/z): [M+H+] calcd for: C25H32NO 362.2484; found: 362.2485; Anal. Calcd. For C₂₅H₃₂ClNO·0.1 H₂O: C, 75.11%; H, 8.12%; N, 3.5%. Found C, 75.00%; H, 7.87%; N, 3.42%; [a]20D −14.1° (c 0.82, CHCl₃).

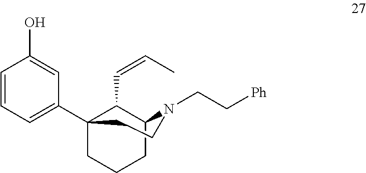 [SMILES: C/C=C\[C@@H]1[C@@H]2CCC[C@@]1(c1cccc(O)c1)CCN2CCc1ccccc1]

3-((1R,5R,9S)-2-Phenethyl-9-((E)-prop-1-en-1-yl)-2-azabicyclo[3.3.1]nonan-5-yl) phenol (Compound 27): Procedure same as Compound 23. Isolated as a light green foam (474 mg, 95% yield). The HCl salt of Compound 27 was formed in iPrOH with 37% HCl (0.1 mL) and recrystallized from ethanol to give a white solid: mp 277-281; 1H-NMR (400 MHZ; CD3OD): δ 7.38-7.33 (m, 1H), 7.29 (tt, J=9.1, 4.7 Hz,), 7.12-7.09 (m,), 6.84-6.80 (m, 1H), 6.61 (dd, J=8.0, 2.1 Hz,), 5.61-5.53 (m,), 5.41-5.36 (m,), 3.72-3.63 (m, 1H), 3.50 (dd, J=10.4, 6.5 Hz,), 3.17-3.13 (m, 1H), 2.34-2.21 (m, 1H), 2.19-1.91 (m, 1H), 1.76 (dd, J=6.9, 1.1 Hz, 1H); 13C NMR (101 MHz; CD3OD): δ 158.5, 149.8, 137.7, 130.3, 130.00, 129.91, 129.5, 128.3, 127.4, 117.8, 114.2, 113.8, 61.1, 56.8, 51.1, 41.9, 39.8, 37.8, 31.8, 29.2, 21.1, 18.7, 13.8; HRMS-ESI (m/z): [M+H+] calcd for: C25H32NO 362.2484; found 362.2484; Anal. Calcd. For C25H32ClNO:C, 75.45%; H, 8.7%; N, 3.52%. Found C, 75.27%; H, 7.73%; N, 3.44%; [a]20D 15.0° (c 0.86, CHCl₃).

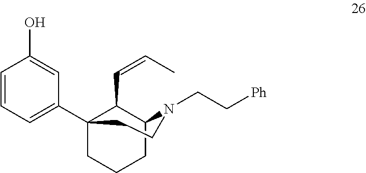 [SMILES: C/C=C\[C@H]1[C@@H]2CCC[C@@]1(c1cccc(O)c1)CCN2CCc1ccccc1]

3-((1R,5R,9R)-2-Phenethyl-9-((E)-prop-1-en-1-yl)-2-azabicyclo[3.3.1]nonan-5-yl) phenol (Compound 26): Procedure same as Compound 22. Isolated as a white foam (1.5 g, 36% yield). The HCl salt of Compound 26 was formed in iPrOH with 37% HCl (0.1 mL) and recrystallized from ethanol to give a white solid: mp 269-274; 1H NMR (400 MHZ; CD3OD): δ 7.35-7.24 (m, 5H), 7.10-7.06 (m, 1H), 6.77-6.68 (m, 2H), 6.61-6.57 (m, 1H), 5.71-5.63 (m, 1H), 5.36-5.30 (m, 1H), 3.73-3.66 (m, 2H), 3.59-3.47 (m, 3H), 3.36 (td, J=12.0, 5.3 Hz, 1H), 3.11 (td, J=12.0, 5.5 Hz, 1H), 2.94-2.87 (m, 1H), 2.55-2.47 (m, 1H), 2.44-2.35 (m, 2H), 2.18-2.02 (m, 2H), 1.99-1.80 (m, 3H), 1.81-1.74 (m, 3H). 13C NMR (101 MHz; CD₃OD): δ 158.5, 150.0, 137.4, 131.0, 130.3, 130.03, 129.9, 128.4, 127.7, 117.5, 114.1, 113.6, 60.4, 56.5, 51.8, 42.4, 42.2, 37.8, 31.5, 29.1, 23.9, 22.1, 13.7. HRMS-ESI (m/z): [M+H+] calcd for: C25H32NO 362.2484; found 362.2487. Anal. Calcd. For C₂₅H₃₂ClNO·0.15 H₂O: C, 74.94%; H, 8.13%; N, 3.5%. Found C, 74.8%; H, 7.85%; N, 3.36%; [a]20D 22.3° (c 1.0, CHCl₃).

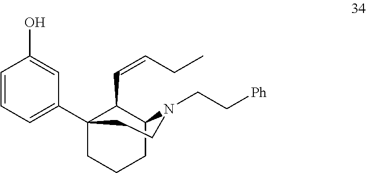 [SMILES: CC/C=C\[C@H]1[C@@H]2CCC[C@@]1(c1cccc(O)c1)CCN2CCc1ccccc1]

3-((1S,5R,9R)-9-((E)-but-1-en-1-yl)-2-phenethyl-2-azabicyclo[3.3.1]nonan-5-yl) phenol (Compound 34): In an oven-dried round-bottom flask, Compound 32 (545 mg, 1 equiv, 1.4 mmol) was suspended in dichloromethane (15 mL) and the mixture was cooled to −78° C. Tribromoborane (700 mg, 265 μL, 2 equiv, 2.8 mmol) was added to drop-wise and the reaction was stirred at −78° C. for 15 min. The reaction mixture was allowed to warm to room temperature and stirred 2 hours. Upon completion, the reaction mixture was cooled to 0° C. and quenched with 7 mL MeOH dropwise and stirred for 30 minutes. Subsequently, 10 mL 1N HCl was added, and the reaction mixture was distilled at 100° C. for 1 hour. The reaction mixture was then cooled to 0° C. and made basic (>10.5) with NH₄OH and extracted with 9:1 CHCl₃:MeOH. The combined organic layers were washed with water and brine, dried with sodium sulfate and concentrated. Purification by silica gel column chromatography 0-60% EtOAc:Hexanes. Compound 34 was isolated as a white foam (294 mg, 56% yield) as the less polar fraction. The HCl salt of Compound 34 was formed in iPrOH (1 mL) with 37% HCl (0.1 mL) and recrystallized from hot ethanol (4 mL) to give a white solid: mp 264-267° C. 1H-NMR (400 MHZ; CD₃OD): δ 7.37-7.26 (m, 5H), 7.10 (t, J=8.0 Hz, 1H), 6.75 (d, J=7.9 Hz, 1H), 6.71 (s, 1H), 6.61 (dd, J=8.0, 2.1 Hz, 1H), 5.59-5.53 (m, 1H), 5.30 (dd, J=10.7, 9.4 Hz, 1H), 3.76-3.68 (m, 2H), 3.56 (dt, J=13.6, 7.0 Hz, 2H), 3.49-3.47 (m, 1H), 3.38 (td, J=12.1, 5.2 Hz, 1H), 3.13 (td, J=12.1, 5.5 Hz, 1H), 2.92 (ddd, J=12.3, 11.6, 5.3 Hz, 1H), 2.60-2.51 (m, 1H), 2.42-2.35 (m, 2H), 2.28-1.83 (m, 7H), 1.04 (t, J=7.5 Hz, 3H). 13C NMR (101 MHz; CD3OD): δ 158.5, 150.0, 138.5, 137.4, 130.3, 130.0, 129.9, 128.4, 125.9, 117.6, 114.1, 113.7, 60.9, 56.5, 51.8, 42.7, 42.1, 37.8, 31.5, 29.0, 23.9, 22.16, 22.08, 14.2. HRMS-ESI (m/z): [M+H+] calcd for: C₂₆H₃₄NO 376.2640; found: 376.2644; Anal. Calcd. For C₂₆H₃₄ClNO·0.1 H2O: C, 75.46%; H, 8.33%; N, 3.38%; Found C, 75.47%; H, 8.2%; N, 3.28%; [a]20D° 12.9 (c 0.79, CHCl3).

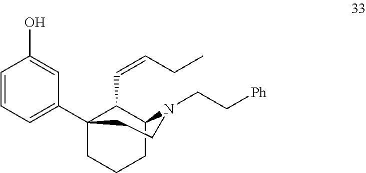 [SMILES: CC/C=C\[C@@H]1[C@@H]2CCC[C@@]1(c1cccc(O)c1)CCN2CCc1ccccc1]

3-((1S,5R,9S)-9-((E)-but-1-en-1-yl)-2-phenethyl-2-azabicyclo[3.3.1]nonan-5-yl) phenol (Compound 33): In an oven-dried round-bottom flask, Compound 32 (545 mg, 1 equiv, 1.4 mmol) was suspended in dichloromethane (15 mL) and the mixture was cooled to −78° C. Tribromoborane (700 mg, 265 μL, 2 equiv, 2.8 mmol) was added to drop-wise and the reaction was stirred at −78° C. for 15 minutes. The reaction mixture was allowed to warm to room temperature and stirred 2 hours. Upon completion, the reaction mixture was cooled to 0° C. and quenched with 7 mL MeOH dropwise and stirred for 30 min. subsequently, 10 mL 1N HCl was added, and the reaction mixture was distilled at 100° C. for 1 hour. The reaction mixture was then cooled to 0° C. and made basic (>10.5) with NH₄OH and extracted with 9:1 CHCl3:MeOH. The combined organic layers were washed with water and brine, dried with sodium sulfate and concentrated. Purification by silica gel column chromatography 0-60% EtOAc:Hexanes. Compound 33 was isolated as a white foam (347 mg, 66% yield) as the more polar fraction. The HCl salt of Compound 33 was formed in iPrOH (1 mL) with 37% HCl (0.1 mL) and recrystallized from hot ethanol (4 mL) to give a white solid: mp 259-262° C. 1H-NMR (400 MHZ; CD₃OD): δ 7.35 (t, J=6.5 Hz, 4H), 7.28 (dq, J=8.6, 4.3 Hz, 1H), 7.10 (t, J=7.9 Hz, 1H), 6.84-6.81 (m, 2H), 6.61 (dd, J=8.0, 2.3 Hz, 1H), 5.43 (dt, J=10.9, 7.2 Hz, 1H), 5.32 (dd, J=10.7, 9.3 Hz, 1H), 3.68-3.62 (m, 3H), 3.59 (d, J=13.4 Hz, 1H), 3.49 (t, J=8.4 Hz, 2H), 3.15 (dd, J=10.3, 5.8 Hz, 2H), 2.38-1.93 (m, 10H), 0.97 (t, J=7.5 Hz, 3H). 13C NMR (101 MHz; CD₃OD): δ 158.5, 149.8, 137.7, 137.1, 130.2, 129.9, 129.9, 128.3, 125.6, 117.8, 114.2, 113.9, 61.6, 56.8, 51.1, 42.3, 39.7, 37.8, 31.7, 29.1, 22.2, 21.1, 18.6, 14.3. HRMS-ESI (m/z): [M+H+] calcd for: C₂₆H₃₄NO 376.2640; found: 376.2642; Anal. Calcd. For C26H34ClNO:C, 75.79%; H, 8.32%, N, 3.4%. Found C26H34ClNO:C, 75.89%; H, 8.18%; N, 3.47%; [a]20D° 4.1 (c 1.08, CHCl₃).

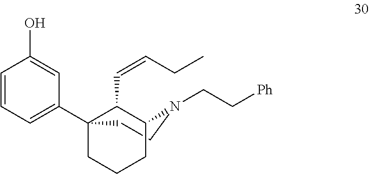 [SMILES: CC/C=C\[C@@H]1[C@H]2CCC[C@]1(c1cccc(O)c1)CCN2CCc1ccccc1]

3-((1R,5S,9S)-9-((E)-but-1-en-1-yl)-2-phenethyl-2-azabicyclo[3.3.1]nonan-5-yl) phenol (Compound 30): In an oven-dried round-bottom flask, Compound 28 (825 mg, 1 equiv, 1.4 mmol) was suspended in dichloromethane (20 mL) and the mixture was cooled to −78° C. Tribromoborane (1.06 g, 402 μL, 2 equiv, 4.23 mmol) was added to drop-wise and the reaction was stirred at −78° C. for 15 minutes. The reaction mixture was allowed to warm to room temperature and stirred 2 hours. Upon completion, the reaction mixture was cooled to 0° C. and quenched with 10 mL MeOH dropwise and stirred for 30 minutes. subsequently, 15 mL 1N HCl was added, and the reaction mixture was distilled at 100° C. for 1 hour. The reaction mixture was then cooled to 0° C. and made basic (>10.5) with NH₄OH and extracted with 9:1 CHCl₃:MeOH. The combined organic layers were washed with water and brine, dried with sodium sulfate and concentrated. Purification by silica gel column chromatography 0-60% EtOAc:Hexanes. Compound 30 was isolated as a white foam (500 mg, 50% yield) as the less polar fraction. The HCl salt of Compound 30 was formed in iPrOH (1.5 mL) with 37% HCl (0.15 mL) and recrystallized from hot ethanol (5 mL) to give a white solid: mp 262-265° C. 1H NMR (400 MHZ; CDCl₃): δ 7.38-7.25 (m, 5H), 7.14-7.07 (m, 1H), 6.79-6.74 (m, 1H), 6.73-6.69 (m, 1H), 6.64-6.60 (m, 1H), 5.60-5.54 (m, 1H), 5.36-5.27 (m, 1H), 3.77-3.67 (m, 2H), 3.62-3.52 (m, 2H), 3.52-3.45 (m, 1H), 3.43-3.33 (m, 1H), 3.17-3.09 (m, 1H), 2.96-2.87 (m, 1H), 2.59-2.49 (m, 1H), 2.46-2.34 (m, 2H), 2.30-1.82 (m, 8H), 1.09-1.00 (m, 3H). 13C NMR (101 MHz; CD3OD): δ 158.5, 150.0, 138.5, 137.4, 130.3, 130.0, 129.9, 128.4, 125.9, 117.6, 114.1, 113.7, 60.9, 56.5, 51.8, 42.7, 42.1, 37.8, 31.5, 29.0, 23.9, 22.2, 22.1, 14.2. HRMS-ESI (m/z): [M+H+] calcd for: C₂₆H₃₄NO 376.2640; found: 376.2641; Anal. Calcd. For C₂₆H₃₄ClNO·0.1 H₂O·0.1 C₂H₆O: C, 75.21%; H, 8.38%; N, 3.35%. Found C, 75.23%; H, 8.4%; N, 3.38%; [a]20D°-14.2 (c 0.8, CHCl₃).

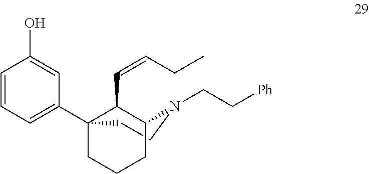 [SMILES: CC/C=C\[C@H]1[C@H]2CCC[C@]1(c1cccc(O)c1)CCN2CCc1ccccc1]

3-((1R,5S,9R)-9-((E)-but-1-en-1-yl)-2-phenethyl-2-azabicyclo[3.3.1]nonan-5-yl) phenol (Compound 29): In an oven-dried round-bottom flask, Compound 28 (825 mg, 1 equiv, 1.4 mmol) was suspended in dichloromethane (20 mL) and the mixture was cooled to −78° C. Tribromoborane (1.06 g, 402 μL, 2 equiv, 4.23 mmol) was added to drop-wise and the reaction was stirred at −78° C. for 15 minutes. The reaction mixture was allowed to warm to room temperature and stirred 2 hours. Upon completion, the reaction mixture was cooled to 0° C. and quenched with 10 mL MeOH dropwise and stirred for 30 minutes. subsequently, 15 mL 1N HCl was added, and the reaction mixture was distilled at 100° C. for 1 hour. The reaction mixture was then cooled to 0° C. and made basic (>10.5) with NH₄OH and extracted with 9:1 CHCl3:MeOH. The combined organic layers were washed with water and brine, dried with sodium sulfate and concentrated. Purification by silica gel column chromatography 0-60% EtOAc:Hexanes. Compound 29 was isolated as a white foam (500 mg, 50% yield) as the less polar fraction. The HCl salt of Compound 29 was formed in iPrOH (1.5 mL) with 37% HCl (0.15 mL) and recrystallized from hot ethanol (5 mL) to give a white solid: mp 265-268° C. 1H-NMR (400 MHZ; CD3OD): δ 7.33 (d,)=4.3 Hz, 4H), 7.29-7.23 (m, 1H), 7.08 (t, J=7.9 Hz, 1H), 6.82-6.78 (m, 2H), 6.59 (dd, J=7.9, 1.7 Hz, 1H), 5.45-5.38 (m, 1H), 5.32-5.27 (m, 1H), 3.65-3.56 (m, 4H), 3.49-3.45 (m, 2H), 3.12 (dd, J=10.6, 5.9 Hz, 2H), 2.35-1.90 (m, 11H), 0.95 (t, J=7.5 Hz, 3H). 13C NMR (100 MHz; CD₃OD): δ 158.5, 149.8, 137.7, 137.1, 130.2, 129.99, 129.91, 128.3, 125.6, 117.8, 114.2, 113.9, 61.6, 56.8, 51.1, 42.3, 39.7, 37.8, 31.8, 29.1, 22.2, 21.1, 18.6, 14.3. HRMS-ESI (m/z): [M+H⁺] calcd for: C₂₆H₃₄NO 376.2640; found: 376.2642; Anal. Calcd. For C₂₆H₃₄ClNO·0.05 C₂H₆O: calc. C: 75.66%; H: 8.34%; N: 3.38%; found C: 75.65%; H: 8.31%; N: 3.34%; [a]20D°-4.1 (c 0.96, CHCl₃).

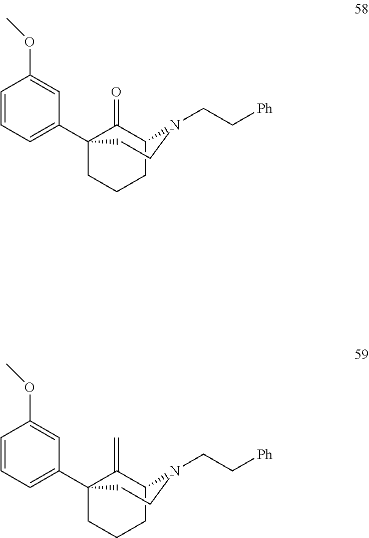 [SMILES: COc1cccc([C@]23CCC[C@H](C2=O)N(CCc2ccccc2)CC3)c1 ; C=C1[C@H]2CCC[C@]1(c1cccc(OC)c1)CCN2CCc1ccccc1]

(1R,5S)-(+)-5-(3-Methoxyphenyl)-9-methylene-2-phenethyl-2-azabicyclo[3.3.1]nonane (Compound 59): The starting ketone (Compound 58) (1.85 g, 1 equiv, 5.29 mmol) was diluted in THF (30.0 mL) and cooled to 0° C. Tebbe's Reagent (1.51 g, 10.6 mL, 0.5 molar, 1 equiv, 5.29 mmol) was slowly added. The resulting mixture was stirred at 0° C. for 1 hour and then slowly warmed up to room temperature for an additional 2 h. After 2 hours, the reaction was still not complete by TLC so ran an additional 2 h checking TLC every 30 minutes. The reaction was cooled to 0° C. and 50 mL of Et2O was added. The reaction was quenched carefully with 1.8 N NaOH. A very vigorous gas evolution took place and a thick red/orange precipitate formed. Magnesium sulfate was added, and the mixture was allowed to stir an additional 5 minutes. The solids were filtered and washed with EtOAc. Purification by silica gel flash chromatography 1-5% CMA in CHCl₃ gave a yellow oil (1.28 g, 70% yield). The product was identical to the standard.

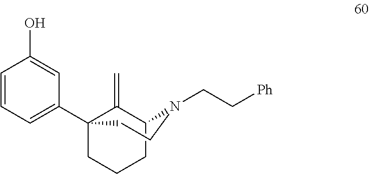 [SMILES: C=C1[C@H]2CCC[C@]1(c1cccc(O)c1)CCN2CCc1ccccc1]

3-((1R,5S)-9-methylene-2-phenethyl-2-azabicyclo[3.3.1]nonan-5-yl) phenol (Compound 60): In an oven-dried round-bottom flask, Compound 59 (420 mg, 1 equiv, 1.21 mmol) was suspended in dichloromethane (15 mL) and the mixture was cooled to −78° C. Tribromoborane (229 μL, 2 equiv, 2.42 mmol) was added to drop-wise and the reaction was stirred at −78° C. The reaction mixture was allowed to warm to room temperature and stirred 2 hours. Upon completion, the reaction mixture was cooled to 0° C. and quenched with 7 mL MeOH dropwise and stirred for 30 minutes. 10 mL 1N HCl was added, and the reaction mixture was distilled at 100° C. for 1 hours. The reaction mixture was then cooled to 0° C. and made basic (>10.5) with NH₄OH and extracted with 9:1 CHCl₃:MeOH. The combined organic layers were washed with water and brine, dried with sodium sulfate and concentrated. The product was converted to the oxalate salt: white solid, 200 mg, 40% yield. The product was identical by 1H NMR to the standard.

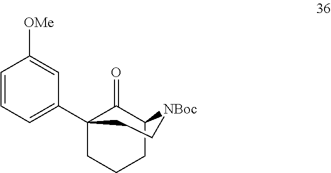 [SMILES: COc1cccc([C@@]23CCC[C@@H](C2=O)N(C(=O)OC(C)(C)C)CC3)c1]

(1S,5S)-5-(3-Methoxyphenyl)-2-azabicyclo[3.3.1]nonan-9-one (Compound 36): In a flask, Compound 35 (3.32 g, 13.5 mmol, 1 equiv) and DMAP (165 mg, 1.35 mmol, 0.100 equiv) were dissolved in DCM (34 mL). Contents were cooled to 0° C. and TEA (2.83 mL, 20.3 mmol, 1.5 equiv) was added followed by Boc₂O (4.66 mL, 20.3 mmol, 1.5 equiv). After stirring overnight, a spatula tip's worth of imidazole was added, and the solution stirred for another hour to quench excess Boc₂O. The contents were transferred to a separatory funnel and washed with dilute HCl and water, the aqueous layer was backextracted with DCM (2×) and the organic layers dried over Na₂SO₄. Removal of the solvent in vacuo followed by purification by flash chromatography with 0 to 40% EtOAc/hexane afforded a viscous yellow oil (3.69 g, 79%). 1H-NMR (400 MHZ; CDCl₃): δ 7.30-7.26 (m, 1H), 6.82-6.76 (m, 3H), 4.31 (s, 1H), 4.24-4.19 (m, 1H), 3.80 (s, 3H), 3.22-3.15 (m, 1H), 2.52 (ddd, J=14.4, 11.4, 5.5 Hz, 2H), 2.39-2.31 (m, 2H), 2.19 (dd, J=8.2, 2.9 Hz, 2H), 1.80-1.72 (m, 2H), 1.49 (s, 9H). 13C-NMR (101 MHz; CDC (3): δ 211.8, 170.9, 159.2, 154.6, 145.6, 129.0, 119.3, 111.3, 80.3, 63.6, 55.1, 52.8, 41.0, 40.6, 38.1, 35.5, 28.4, 17.6 HRMS [M+Na+] calc: 368.1838, found: 368.1833 [a]20D −37.1° (c 1.08, CHCl₃)

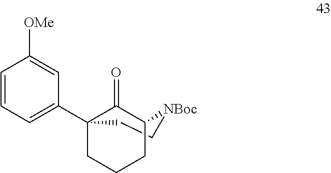 [SMILES: COc1cccc([C@]23CCC[C@H](C2=O)N(C(=O)OC(C)(C)C)CC3)c1]

(1R,5R)-5-(3-Methoxyphenyl)-2-azabicyclo[3.3.1]nonan-9-one (Compound 43): Ran analogously to Compound 36 on a 3.88 g scale and obtained a yellow oil (4.26 g, 78%). 1H-NMR (400 MHz; CDCl₃): δ 7.28 (d, J=8.0 Hz, 1H), 6.79 (ddd, J=11.6, 6.0, 2.2 Hz, 3H), 4.31 (s, 1H), 4.23-4.18 (m, 1H), 3.80 (s, 3H), 3.21-3.15 (m, 1H), 2.52 (ddd, J=14.3, 11.4, 5.5 Hz, 2H), 2.38-2.31 (m, 2H), 2.19 (dd, J=8.2, 2.8 Hz, 2H), 2.04 (s,), 1.77-1.71 (m, 2H). 13C-NMR (101 MHz; CDCl₃): δ 211.8, 159.3, 154.6, 145.6, 129.0, 119.4, 113.5, 111.3, 80.3, 63.6, 55.1, 52.8, 41.0, 40.6, 38.1, 28.4, 17.6 HRMS [M+Na+] calc: 368.1838, found: 368.1833 [a]20D+42.9° (c 1.13, CHCl₃)

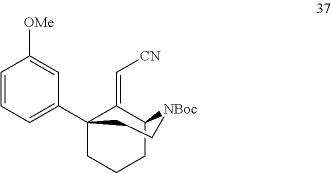 [SMILES: COc1cccc([C@@]23CCC[C@@H](/C2=C\C#N)N(C(=O)OC(C)(C)C)CC3)c1]

(Z)-2-((1S,5S)-5-(3-Methoxyphenyl)-2-phenethyl-2-azabicyclo[3.3.1]nonan-9-ylidene) acetonitrile (Compound 37): To a flame-dried flask was added NaH (452 mg, 11.3 mmol, 3 equiv, 60% dispersion in mineral oil). The solid was washed with hexane 3× under nitrogen before THF (10 mL) was added. To this suspension diethyl (cyanomethyl)phosphonate (1.83 mL, 11.3 mmol, 3 equiv) was added dropwise. Once gas evolution ceased and a clear solution formed, Compound 36 as a solution in THF (7 mL) was added and the reaction mixture heated to reflux overnight. After cooling to room temperature, EtOH was added dropwise to quench excess NaH and the contents were concentrated in vacuo. The residual oil was purified by chromatography with 0 to 20% EtOAc/hexane to yield a white solid (961 mg, 69%). 1H-NMR (400 MHZ; CDCl₃): δ 7.30 (t, J=7.9 Hz, 1H), 6.82 (dd, J=8.4, 5.7 Hz, 3H), 5.16-5.10 (m, 1H), 4.61 (s, 1H), 4.09-4.06 (m, 1H), 3.81 (s, 3H), 3.21-3.13 (m, 1H), 2.42-2.39 (m, 1H), 2.28 (dd, J=10.4, 5.2 Hz, 2H), 2.07 (dq, J=17.2, 6.6 Hz, 3H), 1.73 (dd, J=7.2, 6.1 Hz, 1H), 1.51 (s, 9H). 13C-NMR (101 MHZ; CDCl₃): δ 171.6, 159.8, 155.0, 147.4, 129.8, 119.6, 116.1, 113.9, 111.6, 94.9, 80.6, 55.6, 55.4, 45.8, 40.5, 39.9, 38.5, 34.2, 28.6, 18.1 HRMS [M+H+] calc: 391.1998, found: 391.1997, MP=124-126° C., [a]20D −44.3° (c 1.10, CHCl₃).

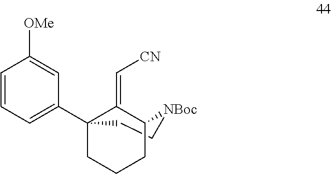 [SMILES: COc1cccc([C@]23CCC[C@H](/C2=C\C#N)N(C(=O)OC(C)(C)C)CC3)c1]

(Z)-2-((1R,5R)-5-(3-Methoxyphenyl)-2-phenethyl-2-azabicyclo[3.3.1]nonan-9-ylidene) acetonitrile (Compound 44): Ran analogously to Compound 37 on a 4.26 g scale and obtained a white solid (2.91 g, 64%). 1H-NMR (400 MHZ; CDCl₃): δ 7.23 (t, J=7.8 Hz, 1H), 6.76 (t, J=7.4 Hz, 3H), 5.07 (d, J=24.1 Hz, 1H), 4.55 (s, 1H), 4.03-3.94 (m, 1H), 3.74 (s, 3H), 3.11 (dd, J=15.2, 7.7 Hz, 1H), 2.35 (d, J=13.1 Hz, 1H), 2.22 (dd, J=9.9, 5.1 Hz, 2H), 2.06-1.97 (m, 3H), 1.67 (d, J=6.0 Hz, 1H), 1.45 (s, 10H). 13C-NMR (101 MHz; CDC (3): δ 171.4, 159.6, 154.9, 147.2, 129.6, 119.4, 115.9, 113.8, 111.4, 94.7, 80.4, 55.5, 55.2, 45.6, 40.4, 39.7, 38.3, 34.1, 28.4, 17.9 HRMS [M+Na+] calc: 391.1998, found: 391.1998, MP=123-126° C., [a]20D+44.0° (c 1.02, CHCl₃).

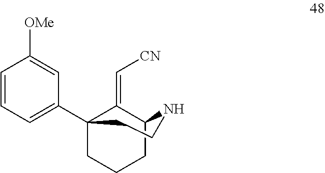 [SMILES: COc1cccc([C@]23CCC[C@H](NCC2)/C3=C\C#N)c1]

(Z)-2-((1S,5S)-5-(3-Methoxyphenyl)-2-azabicyclo[3.3.1]nonan-9-ylidene) acetonitrile (Compound 48): A solution of Compound 37 (500 mg, 1.36 mmol, 1 equiv) in DCM (4.6 mL) was prepared and cooled to 0° C. before TFA (2.09 mL, 27.1 mmol, 20 equiv) was added dropwise. After 30 minutes, TLC showed no evidence for starting material. The pH was adjusted to 9 with NH₄OH and the aqueous layer was extracted with CHCl3 (3×). The combined organic layers were dried over Na2SO4, filtered, and concentrated down to a golden oil that was used directly in the next step without purification. 1H-NMR (400 MHZ; CDCl₃): δ 7.27-7.23 (m, 1H), 6.84 (d, J=7.9 Hz, 1H), 6.80 (t, J=2.0 Hz, 1H), 6.76 (dd, J=8.1, 2.3 Hz, 1H), 4.58 (s, 1H), 4.29 (s, 1H), 3.76 (s, 3H), 3.41 (ddd, J=13.6, 8.7, 4.8 Hz, 1H), 2.93 (dt, J=13.1, 6.3 Hz, 1H), 2.37-2.22 (m, 4H), 2.15-2.07 (m, 4H), 1.88-1.78 (m, 1H), 1.72 (dtd, J=11.2, 5.6, 2.9 Hz, 1H). 13C-NMR (101 MHz; CDC (3): δ 175.3, 159.5, 147.1, 129.4, 119.5, 116.5, 113.8, 111.3, 92.2, 55.2, 53.9, 46.1, 41.8, 41.5, 39.5, 34.6, 20.0 HRMS [M+H+] calc: 269.1654, found: 269.1653 [a]20D −69.2° (c 1.11, CHCl₃)

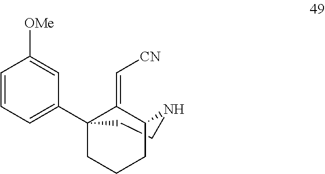 [SMILES: COc1cccc([C@@]23CCC[C@@H](NCC2)/C3=C\C#N)c1]

(Z)-2-((1R,5R)-5-(3-Methoxyphenyl)-2-azabicyclo[3.3.1]nonan-9-ylidene) acetonitrile (Compound 49): Ran analogously to Compound 48 on a 2.50 g scale, obtained a yellow oil that was used directly in the next step without purification. 1H-NMR (400 MHZ; CDCl₃): δ 7.24 (t, J=8.0 Hz, 1H), 6.83 (d, J=7.9 Hz, 1H), 6.79 (t, J=2.0 Hz, 1H), 6.76 (dd, J=8.1, 2.4 Hz, 1H), 4.57 (s, 1H), 4.28 (t, J=3.4 Hz, 1H), 3.76 (s, 3H), 3.40 (ddd, J=13.6, 8.7, 4.8 Hz, 1H), 2.92 (dt, J=13.1, 6.3 Hz, 1H), 2.35-2.22 (m, 3H), 2.14-2.06 (m, 4H), 1.87-1.77 (m, 1H), 1.71 (dddd, J=11.3, 8.4, 5.6, 2.8 Hz, 1H). ¹³C-NMR (101 MHz; CDC (3): δ 175.4, 159.5, 147.1, 129.3, 119.5, 116.6, 113.8, 111.3, 92.1, 55.2, 53.9, 46.1, 41.9, 41.5, 39.5, 34.6, 20.0 HRMS [M+H+] calc: 269.1654, found: 269.1654 [a]20D+66.0° (c 0.98, CHCl₃).

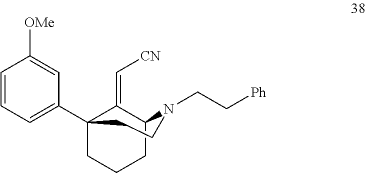 [SMILES: COc1cccc([C@@]23CCC[C@@H](/C2=C\C#N)N(CCc2ccccc2)CC3)c1]

(Z)-2-((1S,5S)-5-(3-methoxyphenyl)-2-phenethyl-2-azabicyclo[3.3.1]nonan-9-ylidene) acetonitrile (Compound 38): To a flask was added K2CO3 (375 mg, 2.71 mmol, 2 equiv), Compound 48 (364 mg, 1.36 mmol, 1 equiv), phenethyl bromide (278 uL, 2.03 mmol, 1.5 equiv), and MeCN (3.2 mL). After heating the suspension to reflux overnight, the solids were filtered off and washed with MeCN. The filtrate was concentrated down and purified by chromatography with 0 to 20% EtOAc/hexane to obtain a colorless oil (449 mg, 89%). 1H-NMR (400 MHZ; CDCl₃): δ 7.30 (td, J=7.6, 4.5 Hz, 4H), 7.24 (dt, J=6.9, 5.4 Hz, 3H), 6.90-6.87 (m, 1H), 6.85 (t, J=2.1 Hz, 1H), 6.81 (dd, J=8.0, 2.3 Hz, 1H), 4.63 (s, 1H), 4.11 (t, J=3.2 Hz, 1H), 3.87 (t, J=6.6 Hz, 1H), 3.82 (s, 3H), 3.15 (ddd, J=11.8, 6.7, 5.0 Hz, 1H), 2.94-2.85 (m, 5H), 2.76 (ddd, J=11.9, 8.1, 5.1 Hz, 1H), 2.39 (dt, J=13.4, 6.4 Hz, 1H), 2.25-2.06 (m, 6H), 1.69 (dtd, J=11.1, 5.5, 2.7 Hz, 1H), 1.63-1.54 (m, 1H). 13C-NMR (101 MHz; CDC (3): δ 173.9, 159.7, 147.6, 140.3, 138.6, 129.5, 129.2, 128.92, 128.73, 128.5, 126.6, 126.2, 119.8, 116.9, 114.1, 111.4, 93.6, 63.8, 60.6, 59.0, 55.4, 48.4, 46.0, 39.7, 39.3, 38.9, 34.7, 32.8, 19.6 HRMS [M+H+] calc: 373.2280, found: 373.2281, [a]20D −11.6° (c 1.00, CHCl₃).

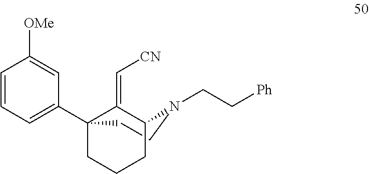 [SMILES: COc1cccc([C@]23CCC[C@H](/C2=C\C#N)N(CCc2ccccc2)CC3)c1]

(Z)-2-((1R,5R)-5-(3-methoxyphenyl)-2-phenethyl-2-azabicyclo[3.3.1]nonan-9-ylidene) acetonitrile (Compound 50): Ran analogously to Compound 38 on a 1.89 g scale, obtained a colorless oil (2.32 g, 88%). 1H-NMR (400 MHZ; CDCl₃): δ 7.31-7.26 (m, 3H), 7.25 (td, J=4.1, 2.5 Hz, 2H), 7.22-7.18 (m, 1H), 6.88-6.86 (m, 1H), 6.84 (t, J=2.0 Hz, 1H), 6.80 (dd, J=8.1, 2.4 Hz, 1H), 4.62 (s, 1H), 4.10 (t, J=3.2 Hz, 1H), 3.80 (s, 3H), 3.13 (ddd, J=11.8, 6.7, 5.0 Hz, 1H), 2.88 (tq, J=12.5, 4.3 Hz, 4H), 2.75 (ddd, J=12.2, 7.8, 4.8 Hz, 1H), 2.37 (dd, J=13.2, 6.7 Hz, 1H), 2.24-2.05 (m, 5H), 1.68 (ddd, J=11.1, 5.4, 2.8 Hz, 1H), 1.59-1.57 (m, 1H). 13C-NMR (101 MHZ; CDCl₃): δ 173.8, 159.5, 147.5, 140.1, 129.4, 128.8, 128.4, 126.0, 119.7, 116.7, 113.9, 111.3, 93.4, 60.4, 58.8, 55.2, 48.3, 45.8, 39.6, 38.8, 34.5, 32.6, 19.5 HRMS [M+H+] calc: 373.2280, found: 373.2275, [a]20D +12.3° (c 1.09, CHCl₃).

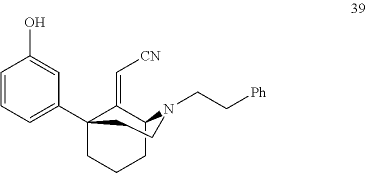 [SMILES: N#C/C=C1\[C@@H]2CCC[C@@]1(c1cccc(O)c1)CCN2CCc1ccccc1]

(Z)-2-((1S,5S)-5-(3-hydroxyphenyl)-2-phenethyl-2-azabicyclo[3.3.1]nonan-9-ylidene) acetonitrile (Compound 39): A solution of Compound 38 (782 mg, 2.10 mmol, 1 equiv) in DCM (47 mL) was prepared and cooled to −78° C. before BBr3 (992 uL, 10.5 mmol, 5 equiv) was added dropwise. After 15 minutes, the cooling bath was removed, and the solution allowed to stir at room temperature for an additional 2 hours. The solvent was removed in vacuo, and the residue taken up in water. The pH was adjusted to 9 with NH₄OH, and the contents stirred for 30 min to break up borane complexes. The aqueous layer was extracted with CHCl₃ (3×) and the combined organic layers were dried over Na₂SO₄. The solids were filtered off, and the filtrate concentrated then purified with 0 to 20% EtOAc/hexane to afford a white foam (487 mg, 65%). Crystallization was performed by taking product (100 mg) and dissolving in minimal aqueous MeOH then adding 2 N HCl in Et2O (2 equiv) to afford a white solid (65 mg, 63% recovery) ¹H-NMR (400 MHZ; CDCl₃): δ 7.32-7.28 (m, 2H), 7.25-7.19 (m, 4H), 6.86-6.84 (m, 1H), 6.78 (t, J=2.1 Hz, 1H), 6.74 (dd, J=7.8, 2.2 Hz, 1H), 4.65 (s, 1H), 4.12 (t, J=3.1 Hz, 1H), 3.16 (ddd, J=11.9, 7.0, 4.9 Hz, 1H), 2.94-2.85 (m, 4H), 2.78 (ddd, J=12.2, 7.4, 5.1 Hz, 1H), 2.39 (dt, J=13.6, 6.5 Hz, 1H), 2.24-2.08 (m, 5H), 1.69 (ddt, J=10.9, 5.4, 2.7 Hz, 1H), 1.63-1.57 (m, 1H). ¹³C-NMR (101 MHz; CDCl₃): δ 173.8, 155.8, 147.8, 140.1, 129.7, 128.9, 128.5, 126.2, 119.7, 116.9, 114.6, 114.1, 93.7, 60.5, 58.9, 48.4, 45.9, 39.7, 38.7, 34.5, 32.5, 19.7 HRMS [M+H+] calc: 359.2123, found: 359.2122, MP=152-153° C., [a]20D −11.0° (c 0.99, CHCl₃). CHN calc. for C₂₄H₂₇ClN₂O·0.15 H₂O: C, 72.49%; H, 6.92%; N, 7.04%; found C, 72.48%; H, 6.90%; N, 7.07%.

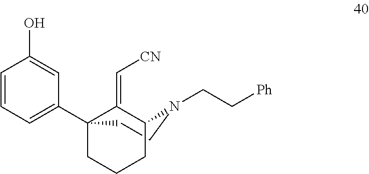 [SMILES: N#C/C=C1\[C@H]2CCC[C@]1(c1cccc(O)c1)CCN2CCc1ccccc1]

(Z)-2-((1R,5R)-5-(3-hydroxyphenyl)-2-phenethyl-2-azabicyclo[3.3.1]nonan-9-ylidene) acetonitrile (Compound 40): Ran analogously to Compound 39 on a 500 mg scale, obtained a white foam (292 g, 61%). Crystallization afforded a white solid (76 mg, 68% recovery). 1H-NMR (400 MHZ; CDCl₃): δ 7.30-7.26 (m, 2H), 7.24-7.17 (m, 4H), 6.84-6.82 (m, 1H), 6.76 (t, J=2.0 Hz, 1H), 6.72 (dd, J=8.0, 1.9 Hz, 1H), 4.64 (s, 1H), 4.12 (dd, J=5.3, 1.8 Hz, 1H), 3.16 (ddd, J=12.0, 7.2, 4.9 Hz, 1H), 2.87 (qd, J=8.8, 5.1 Hz, 4H), 2.77 (ddd, J=12.2, 7.2, 5.2 Hz, 1H), 2.38 (dt, J=13.7, 6.6 Hz, 1H), 2.23-2.07 (m, 5H), 1.71-1.66 (m, 1H), 1.63-1.55 (m, 1H). 13C-NMR (101 MHZ; CDCl₃): δ 173.6, 155.8, 147.5, 139.9, 129.5, 128.7, 128.4, 126.1, 119.4, 116.7, 114.5, 114.0, 93.5, 60.3, 58.7, 48.2, 45.7, 39.5, 38.5, 34.3, 32.2, 19.6 HRMS [M+H+] calc: 359.2123, found: 359.2122, MP=154° C., [a]20D+11.5° (c 1.02, CHCl₃). CHN calc. for C₂₄H₂₇ClN₂O·1.45 H₂O: C, 68.46%; H, 7.16%; N, 6.65%; found C, 68.19%; H, 6.87%; N, 6.44%.

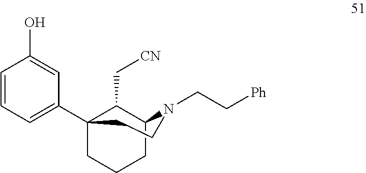 [SMILES: N#CC[C@@H]1[C@@H]2CCC[C@@]1(c1cccc(O)c1)CCN2CCc1ccccc1]

2-((1S,5R,9R)-5-(3-hydroxyphenyl)-2-phenethyl-2-azabicyclo[3.3.1]nonan-9-yl) acetonitrile (Compound 51): Dissolved Compound 39 (170 mg, 0.474 mmol, 1 equiv) in EtOH (33 mL) and passed through the H-cube flow reactor with the following conditions: 70° C., 50 bar H2, 10% Pd/C, 1.2 mL/min flow rate. After a single pass, TLC showed no sm remaining. The reaction mixture was concentrated down and purified by chromatography with 0 to 8% CMA/CHCl₃ to give a white foam (30 mg, 18%). Crystallization was performed by taking product (30 mg) and dissolving in minimal aqueous MeOH then adding 2 N HCl in Et2O (2 equiv) to afford a white solid (23 mg, 70% recovery). 1H-NMR (400 MHZ; CDCl₃): δ 7.31-7.27 (m, 2H), 7.24-7.16 (m, 5H), 6.92 (s, 1H), 6.84 (d, J=7.8 Hz, 1H), 6.75 (dd, J=8.0, 2.2 Hz, 1H), 3.43 (t, J=0.4 Hz, 1H), 3.19-3.17 (m, 1H), 3.13-3.06 (m, 1H), 3.01-2.89 (m, 5H), 2.41-2.35 (m, 1H), 2.22 (dd, J=17.3, 11.9 Hz, 1H), 2.12-1.77 (m, 8H), 1.55-1.50 (m, 1H). ¹³C-NMR (101 MHz; CDCl₃): δ 156.5, 149.1, 139.6, 129.9, 128.7, 128.5, 126.3, 118.7, 117.2, 113.8, 112.9, 57.8, 54.8, 49.5, 42.1, 40.2, 37.6, 33.9, 27.9, 21.1, 18.1, 16.5 HRMS [M+H+] calc: 361.2280, found: 361.2282, MP=63-66° C., [a]20D −2.43° (c 1.30, CHCl₃). CHN calc. for C₂₄H₂₉ClN₂O: C, 72.62%; H, 7.36%; N, 7.06%; found C, 72.69%; H, 7.24%; N, 7.11%.

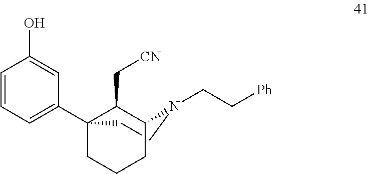 [SMILES: N#CC[C@H]1[C@H]2CCC[C@]1(c1cccc(O)c1)CCN2CCc1ccccc1]

2-((1R,5S,9S)-5-(3-hydroxyphenyl)-2-phenethyl-2-azabicyclo[3.3.1]nonan-9-yl) acetonitrile (Compound 41): Prepared analogously to Compound 51 on a 479 mg scale and obtained a white foam (205 mg, 43%). Salt formation afforded a white solid (180 mg, 80% recovery). 1H-NMR (400 MHZ; CDCl₃): δ 7.32-7.29 (m, 2H), 7.21 (t, J=8.3 Hz, 3H), 6.89 (d, J=8.0 Hz, 1H), 6.82 (s, 1H), 6.71 (dd, J=8.0, 2.2 Hz, 1H), 3.31 (d, J=1.3 Hz, 1H), 3.07-3.04 (m, 2H), 2.94-2.82 (m, 4H), 2.73-2.70 (m, 1H), 2.29-2.22 (m, 2H), 2.13-2.06 (m, 2H), 1.97 (dtd, J=20.4, 10.4, 4.2 Hz, 3H), 1.80 (dt, J=13.2, 2.7 Hz, 2H), 1.50-1.43 (m, 1H). 13C-NMR (101 MHz; CDC (3): δ 156.8, 149.3, 139.9, 130.0, 128.7, 128.5, 126.3, 119.0, 117.0, 114.1, 113.4, 77.51, 77.40, 77.2, 76.9, 57.9, 54.6, 49.4, 42.3, 40.4, 37.7, 34.0, 28.0, 21.3, 18.1, 16.6 HRMS [M+H+] calc: 361.2280, found: 361.2280, MP=63-66° C., [a]20D +3.75° (c 0.99, CHCl₃). CHN calc. for C₂₄H₂₇ClN₂O·0.1 H₂O: C, 72.14%; H, 7.20%; N, 6.99%; found C, 72.29%; H, 7.38%; N, 7.03%.

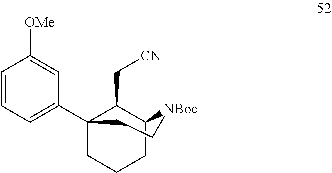 [SMILES: COc1cccc([C@@]23CCC[C@@H]([C@@H]2CC#N)N(C(=O)OC(C)(C)C)CC3)c1]

tert-Butyl (1S,5R,9R)-9-(Cyanomethyl)-5-(3-methoxyphenyl)-2-azabicyclo[3.3.1]nonane-2-carboxylate (Compound 52): Ran analogously to Compound 51 on a 475 mg scale and obtained a white solid (453 mg, 95%). 1H-NMR (400 MHZ; CDCl₃): δ 7.28-7.24 (m, 1H), 6.84 (t, J=6.7 Hz, 1H), 6.80-6.73 (m, 2H), 4.56 (d, J=2.6 Hz, 1H), 4.14-4.09 (m, 1H), 3.78 (s, 3H), 3.53 (td, J=13.0, 6.9 Hz, 1H), 2.43 (d, J=11.7 Hz, 1H), 2.27-2.19 (m, 1H), 2.10-1.69 (m, 10H), 1.47 (s, 9H). ¹³C-NMR (101 MHz; CDCl₃): δ 159.94, 159.89, 155.8, 148.9, 129.9, 119.2, 117.5, 117.1, 112.2, 111.9, 110.87, 110.79, 80.2, 55.2, 49.0, 42.5, 41.8, 40.5, 38.2, 31.2, 28.46, 28.42, 28.33, 27.3, 21.7, 16.6 HRMS [M+Na+] calc: 393.2154, found: 393.2152 MP=143-146° C., [a]20D −63.8° (c 1.09, CHCl₃)

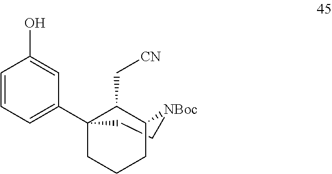 [SMILES: CC(C)(C)OC(=O)N1CC[C@@]2(c3cccc(O)c3)CCC[C@@H]1[C@H]2CC#N]

tert-Butyl (1R,5S,9S)-9-(Cyanomethyl)-5-(3-methoxyphenyl)-2-azabicyclo[3.3.1]nonane-2-carboxylate (Compound 45): Ran analogously to Compound 51 on a 155 mg scale and obtained a white foam (156 mg, >99%). 1H-NMR (400 MHZ; CDCl₃): δ 7.27 (t, J=8.0 Hz, 1H), 6.85 (t, J=6.6 Hz, 1H), 6.80-6.74 (m, 2H), 4.56 (d, J=2.5 Hz, 1H), 4.15-4.09 (m, 1H), 3.79 (s, 3H), 3.54 (td, J=13.0, 6.9 Hz, 1H), 2.43 (d, J=11.6 Hz, 1H), 2.25 (dd, J=16.6, 11.8 Hz, 1H), 2.11-1.70 (m, 10H), 1.48 (s, 9H). 13C-NMR (101 MHz; CDC (3): δ 159.93, 159.89, 155.8, 148.9, 129.9, 119.2, 117.5, 117.1, 112.2, 111.9, 110.87, 110.80, 80.2, 55.2, 49.0, 42.5, 41.8, 40.5, 38.2, 31.2, 28.45, 28.42, 28.33, 27.3, 21.7, 16.6 HRMS [M+Na+] calc: 393.2154, found: 393.2151, MP=144-146° C., [a]20D +64.6° (c 0.99, CHCl₃)

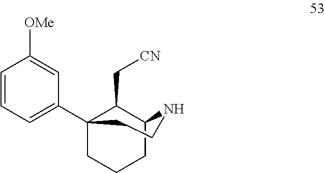 [SMILES: COc1cccc([C@]23CCC[C@H](NCC2)[C@@H]3CC#N)c1]

2-((1S,5R,9R)-5-(3-Methoxyphenyl)-2-azabicyclo[3.3.1]nonan-9-yl) acetonitrile (Compound 53): Combined Compound 52 (453 mg, 1.22 mmol, 1 equiv), DCM (4.5 mL), and TFA (1.88 uL, 24.5 mmol, 20 equiv) in a flask and let stir at room temperature. After 30 min, TLC showed no sm remaining. The reaction was quenched with NaHCO₃, and the aqueous layer with DCM 3× and the combined organic layers were dried over Na₂SO₄. The drying agent was filtered off and the filtrate concentrated down to a golden oil, which was used directly in the next step without purification. 1H-NMR (400 MHZ; CDCl₃): δ 7.24 (s, 1H), 6.90-6.71 (m, 3H), 3.77 (s, 3H), 3.62 (t, J=1.3 Hz, 1H), 3.31 (s, 1H), 2.78-2.71 (m, 1H), 2.34 (d, J=10.3 Hz, 1H), 2.12-1.75 (m, 11H), 1.25-1.23 (m, 1H). 13C-NMR (101 MHz; CDC (3): δ 159.8, 150.3, 129.68, 129.64, 120.1, 117.3, 111.9, 110.5, 55.2, 49.0, 42.5, 42.0, 41.7, 38.5, 34.1, 28.9, 23.0, 16.7 HRMS [M+H+] calc: 271.1810, found: 271.1806 [a]20D −8.62° (c 1.03, CHCl₃)

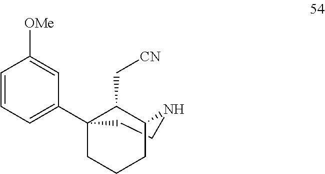 [SMILES: COc1cccc([C@@]23CCC[C@@H](NCC2)[C@H]3CC#N)c1]

2-((1R,5S,9S)-5-(3-Methoxyphenyl)-2-azabicyclo[3.3.1]nonan-9-yl) acetonitrile (Compound 54): Ran analogously to Compound 53 on a 156 mg scale and obtained a golden oil, which was used directly in the next step without purification. 1H-NMR (400 MHZ; CDCl₃): δ 7.24 (t, J=8.0 Hz, 1H), 6.86 (dd, J=7.6, 1.3 Hz, 1H), 6.79 (t, J=2.1 Hz, 1H), 6.72 (dd, J=8.1, 2.4 Hz, 1H), 3.78 (s, 4H), 3.63 (td, J=12.5, 5.5 Hz, 1H), 3.32 (d, J=3.1 Hz, 1H), 3.04 (dd, J=12.4, 7.6 Hz, 1H), 2.76 (dd, J=16.7, 11.6 Hz, 1H), 2.35 (d, J=11.5 Hz, 1H), 2.17-1.72 (m, 12H). ¹³C-NMR (101 MHZ; CDCl₃): δ 159.8, 150.2, 129.7, 120.1, 117.3, 111.9, 110.5, 55.2, 49.0, 42.5, 42.0, 41.7, 38.5, 34.0, 28.9, 22.9, 16.7 HRMS [M+H+] calc: 271.1810, found: 271.1810 [a]20D +9.81° (c 1.03, CHCl₃)

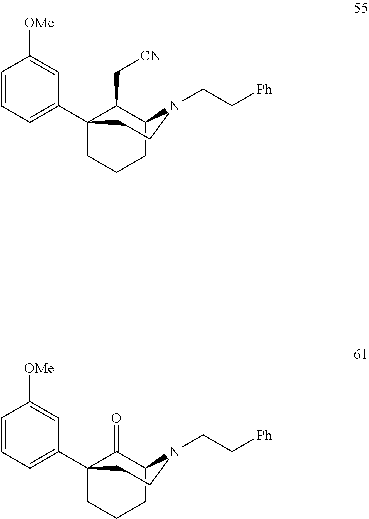 [SMILES: COc1cccc([C@@]23CCC[C@@H]([C@@H]2CC#N)N(CCc2ccccc2)CC3)c1 ; COc1cccc([C@@]23CCC[C@@H](C2=O)N(CCc2ccccc2)CC3)c1]

2-((1S,5R,9R)-5-(3-Methoxyphenyl)-2-phenethyl-2-azabicyclo[3.3.1]nonan-9-yl) acetonitrile (Compound 55): Ran analogously to Compound 61 on a 331 mg scale and obtained a yellow oil (345 mg, 75%). 1H-NMR (400 MHZ; CDC (3): δ 7.29-7.15 (m, 5H), 6.87-6.85 (m, 1H), 6.80 (s, 1H), 6.74 (d, J=8.0 Hz, 1H), 3.79 (s, 3H), 3.15 (s, 1H), 3.09-2.96 (m, 2H), 2.86-2.72 (m, 4H), 2.40 (d, J=10.8 Hz, 1H), 2.34-2.30 (m, 1H), 2.08-1.66 (m, 7H), 1.56-1.49 (m, 2H). 13C-NMR (101 MHZ; CDC (3): δ 159.8, 150.1, 140.6, 129.7, 128.7, 128.3, 125.9, 120.5, 117.4, 112.0, 110.6, 56.5, 55.2, 54.1, 48.3, 43.9, 42.3, 38.5, 34.4, 29.2, 25.6, 23.1, 16.4 HRMS [M+H+] calc: 375.2436, found: 375.2437 [a]20D −9.47° (c 1.05, CHCl₃)

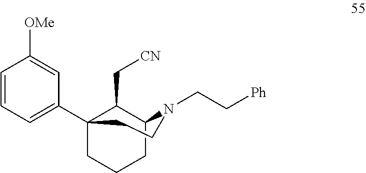 [SMILES: COc1cccc([C@@]23CCC[C@@H]([C@@H]2CC#N)N(CCc2ccccc2)CC3)c1]

2-((1R,5S,9S)-5-(3-Methoxyphenyl)-2-phenethyl-2-azabicyclo[3.3.1]nonan-9-yl) acetonitrile (Compound 55): Ran analogously to Compound 61 on a 114 mg scale and obtained a yellow oil (116 mg, 73%). 1H-NMR (400 MHZ; CDCl₃): δ 7.32-7.18 (m, 6H), 6.88 (d, J=7.9 Hz, 1H), 6.83 (s, 1H), 6.76 (dd, J=8.1, 2.1 Hz, 1H), 3.81 (s, 3H), 3.18 (s, 1H), 3.13-2.99 (m, 2H), 2.89-2.74 (m, 5H), 2.43 (d, J=11.1 Hz, 1H), 2.34 (dd, J=14.0, 3.4 Hz, 1H), 2.10-1.69 (m, 7H), 1.59-1.52 (m, 1H). 13C-NMR (101 MHz; CDC (3): δ 159.8, 150.1, 140.6, 129.7, 128.7, 128.3, 125.9, 120.5, 117.4, 112.0, 110.6, 56.5, 55.2, 54.1, 48.3, 43.8, 42.3, 38.4, 34.4, 29.2, 25.6, 23.1, 16.4 HRMS [M+H+] calc: 375.2436, found: 375.2436 [a]20D +8.76° (c 1.07, CHCl₃)

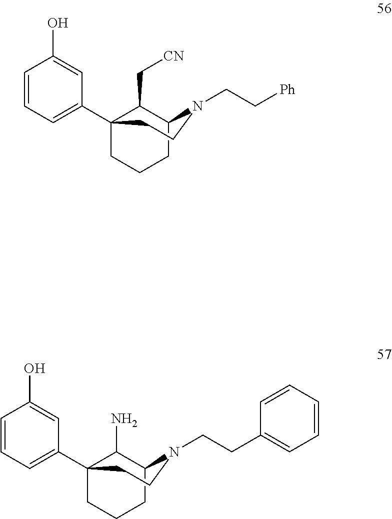 [SMILES: N#CC[C@H]1[C@@H]2CCC[C@@]1(c1cccc(O)c1)CCN2CCc1ccccc1 ; NC1[C@@H]2CCC[C@@]1(c1cccc(O)c1)CCN2CCc1ccccc1]

2-((1S,5R,9R)-5-(3-Hydroxyphenyl)-2-phenethyl-2-azabicyclo[3.3.1]nonan-9-yl) acetonitrile (Compound 56): Ran analogously to Compound 57 on a 345 mg scale and obtained a white foam (156 mg, 47%). Crystallization was performed by taking product (100 mg) and dissolving in minimal aqueous MeOH then adding 2 N HCl in Et₂O (2 equiv) to afford a white solid (82 mg, 74% recovery). 1H-NMR (400 MHZ; CDCl₃): δ 7.28 (t, J=7.4 Hz, 2H), 7.24-7.15 (m, 4H), 6.81-6.77 (m, 2H), 6.71 (dd, J=7.9, 2.1 Hz, 1H), 3.17 (s, 1H), 3.04 (dt, J=12.1, 6.1 Hz, 2H), 2.87-2.74 (m, 4H), 2.39 (d, J=11.2 Hz, 1H), 2.33-2.29 (m, 1H), 2.04-1.64 (m, 7H), 1.56-1.49 (m, 1H). ¹³C-NMR (101 MHz; CDCl₃): δ 160.9, 153.8, 144.3, 133.5, 132.5, 132.1, 129.8, 124.4, 120.2, 117.0, 116.2, 60.2, 58.0, 47.6, 46.0, 42.1, 37.95, 37.94, 32.8, 29.3, 26.8, 20.1 HRMS [M+H⁺] calc: 361.2280, found: 361.2282, MP=73-76° C., [a]²⁰_{D} +14.0° (c 1.01, CHCl₃). CHN calc. for C₂₄H₂₇ClN₂O·0.5 H₂O: C, 71.01%; H, 7.45%; N, 6.90%; found C, 71.10%; H, 7.33%; N, 6.76%.

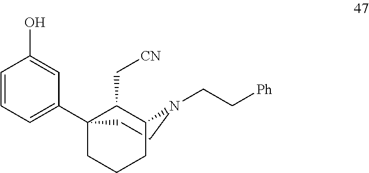 [SMILES: N#CC[C@@H]1[C@H]2CCC[C@]1(c1cccc(O)c1)CCN2CCc1ccccc1]

2-((1R,5S,9S)-5-(3-Hydroxyphenyl)-2-phenethyl-2-azabicyclo[3.3.1]nonan-9-yl) acetonitrile (Compound 47): Ran analogously to Compound 57 on a 116 mg scale and obtained a white foam (53 mg, 77%). Crystallization was performed by taking product (63 mg) and dissolving in minimal aqueous MeOH then adding 2 N HCl in Et₂O (2 equiv) to afford a white solid (65 mg, 63% recovery). 1H-NMR (400 MHZ; CDC (3): δ 7.29-7.24 (m, 2H), 7.18 (dd, J=14.8, 7.1 Hz, 3H), 6.81 (d, J=7.9 Hz, 1H), 6.74 (t, J=2.0 Hz, 1H), 6.67 (dd, J=7.9, 2.2 Hz, 1H), 3.14 (s, 1H), 3.03 (dtd, J=25.4, 12.6, 5.9 Hz, 2H), 2.86-2.72 (m, 5H), 2.38 (d, J=11.1 Hz, 1H), 2.32 (dt, J=14.5, 2.9 Hz, 1H), 2.04-1.65 (m, 7H), 1.57-1.49 (m, 1H), 1.29-1.25 (m, 1H). 13C-NMR (101 MHz; CDCl₃): δ 156.0, 150.4, 140.5, 129.8, 128.6, 128.3, 125.9, 120.5, 117.3, 113.2, 112.4, 56.4, 54.1, 48.2, 43.8, 42.2, 38.4, 34.3, 29.1, 25.5, 23.0, 16.4 HRMS [M+H⁺] calc: 361.2280, found: 361.2283, MP=73-76° C., [a]²⁰_{D} −10.5° (c 0.93, CHC (3). CHN calc. for C₂₄H₂₇ClN2O·0.35 H₂O: C, 71.48%; H, 7.42%; N, 6.95%; found C, 71.49%; H, 7.28%; N, 6.83%.

The C9-alkenyl 5-phenylmorphans synthesized were examined in vitro for their functional activity as percent efficacy (Table 2) based on an inhibition of forskolin-induced CAMP accumulation assay for mu-opioid receptor (MOR), delta-opioid receptor (DOR), kappa-opioid receptor (KOR). The assay can be performed, for example, as described in Ho et al., Sci Signal., 11 (542): eaar4309 (2018) or U.S. Pat. No. 11,352,365, which are incorporated by reference herein in their entireties. Some of these compounds are promising medications for OUD or treatment agents, and others appear to be compounds that will act as analgesics with fewer opioid-like side-effects. In Table 2, agonist data is expressed as EC₅₀+/−SEM (nM) followed by % Eₘₐₓ+/−SEM; and antagonist data is expressed as IC₅₀+/−SEM (nM) followed by % Iₘₐₓ+/−SEM. “N/D” indicates not determined. MOR Antagonist potency (IC50) was determined versus EC₉₀ of fentanyl; degree of antagonism (Imax) normalized to naltrexone. DOR Antagonist potency (IC₅₀) was determined versus EC₅₀ of SNC80; Degree of antagonism (Imax) normalized to naltrexone. KOR Antagonist potency (IC₅₀) was determined versus EC₉₀ of U50488H (a selective KOR agonist); Degree of antagonism (Imax) normalized to nor-BNI. In Table 2, three sets of diastereomeric compounds are listed, plus an additional methoxy compound. The four diastereomers in a set have a common CHN structure and differ only in three-dimensional space. There are three sets of the C9-alkenyl diastereomers (12 compounds). In a fourth set of four diastereomers, not listed in Table 2, there are four C9-cyanomethyl compounds. As in the other three sets, the cyanomethyl compounds have a similar two-dimensional structure, and only differ in three-dimensional space. The diastereomers are all very different in their functional activity, and this could not be anticipated nor deduced from previous work in this area of research.

TABLE 2

| | | | | | | | |
|---|---|---|---|---|---|---|---|
| | | | MOR | | DOR | | KOR |
| | | MOR | Ant- | DOR | Ant- | KOR | Ant- |
| CMP | Structure | Agonist | agonist | Agonist | agonist | Agonist | agonist |

FIRST SET OF FOUR DIASTEROMERS (Vinyl)

| | | | | | | | |
|---|---|---|---|---|---|---|---|
| 19 | 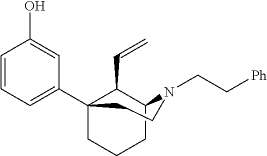 [SMILES: C=C[C@H]1[C@@H]2CCC[C@@]1(c1cccc(O)c1)CCN2CCc1ccccc1] | >10,000 | 3.58 ± 0.72 (130.8 ± 4.5) | >10,000 | 143.65 ± 41.03 (201.4 ± 35.9%) | >10,000 | 28.39 ± 4.24 (104.9 ± 11.2%) |
| 18 | 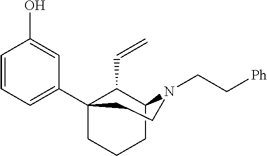 [SMILES: C=C[C@@H]1[C@@H]2CCC[C@@]1(c1cccc(O)c1)CCN2CCc1ccccc1] | 3.94 ± 0.85 (46.6 ± 9.8%) | 50.61 ± 12.13 (70.6 ± 8.0%) | 121.84 ± 47.15 (40.6 ± 6.4%) | N/D | >10,000 | 128.07 ± 48.22 (105.6 ± 4.6%) |
| 13 | 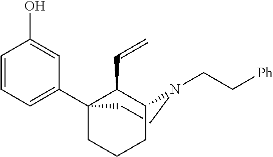 [SMILES: C=C[C@H]1[C@H]2CCC[C@]1(c1cccc(O)c1)CCN2CCc1ccccc1] | 1.44 ± 0.48 (94.7 ± 3.1%) | N/D | >10,000 | 112.85 ± 43.61 (119.1 ± 15.5%) | >10,000 | 74.04 ± 30.49 (97.1 ± 9.2%) |
| 14 | 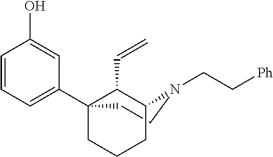 [SMILES: C=C[C@@H]1[C@H]2CCC[C@]1(c1cccc(O)c1)CCN2CCc1ccccc1] | 2.12 ± 0.45 (67.3 ± 6.8%) | 45.55 ± 17.33 (18.8 ± 3.8%) | 57.01 ± 21.43 (22.6 ± 3.3%) | N/D | >10,000 | 19.78 ± 9.71 (101.8 ± 19.1%) |

SECOND SET OF FOUR DIASTEROMERS (Propylene)

| | | | | | | | |
|---|---|---|---|---|---|---|---|
| 26 | 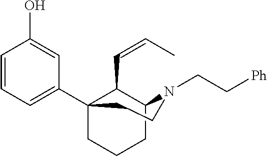 [SMILES: C/C=C\[C@H]1[C@@H]2CCC[C@@]1(c1cccc(O)c1)CCN2CCc1ccccc1] | >10,000 | 2.34 ± 0.20 (157.1 ± 4.0%) | >10,000 | 7.52 ± 2.93 (142.5 ± 23.7%) | >10,000 | 4.02 ± 0.25 (126.7 ± 2.6%) |
| 27 | 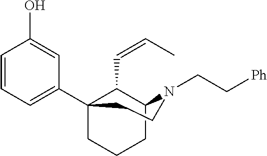 [SMILES: C/C=C\[C@@H]1[C@@H]2CCC[C@@]1(c1cccc(O)c1)CCN2CCc1ccccc1] | 4.66 ± 0.68 (57.5 ± 7.9%) | 45.59 ± 6.71 (39.8 ± 3.9%) | 60.45 ± 3.74 (66.7 ± 4.4%) | N/D | >10,000 | 162.57 ± 37.03 (111.8 ± 2.2%) |
| 22 | 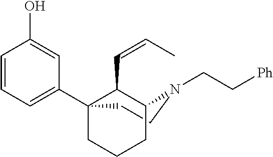 [SMILES: C/C=C\[C@H]1[C@H]2CCC[C@]1(c1cccc(O)c1)CCN2CCc1ccccc1] | 2.61 ± 0.64 (89.5 ± 2.9%) | N/D | >10,000 | 113.23 ± 19.77 (143.3 ± 11.9%) | >10,000 | 35.03 ± 9.72 (111.6 ± 7.9%) |
| 23 | 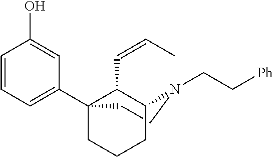 [SMILES: C/C=C\[C@@H]1[C@H]2CCC[C@]1(c1cccc(O)c1)CCN2CCc1ccccc1] | 0.07 ± 0.02 (101 ± 0.2%) | N/D | >10,000 | 9.69 ± 2.23 (74.5 ± 2.3%) | N/D | 237.71 ± 52.57 (114.8 ± 11.2%) |

THIRD SET OF FOUR DIASTEROMERS (Butylene)

| | | | | | | | |
|---|---|---|---|---|---|---|---|
| 34 | 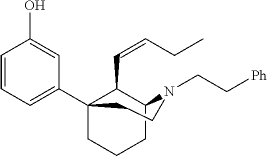 [SMILES: CC/C=C\[C@H]1[C@@H]2CCC[C@@]1(c1cccc(O)c1)CCN2CCc1ccccc1] | >10,000 | 2.37 ± 0.47 (167.8 ± 10.6%) | >10,000 | 8.22 ± 2.44 (211.5 ± 24.7%) | >10,000 | 0.79 ± 0.28 (98.2 ± 2.3%) |
| 33 | 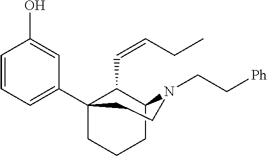 [SMILES: CC/C=C\[C@@H]1[C@@H]2CCC[C@@]1(c1cccc(O)c1)CCN2CCc1ccccc1] | >10,000 | 20.01 ± 10.67 (129.4 ± 3.4%) | >10,000 | 520.36 ± 143.86 (226.1 ± 14.0%) | >10,000 | 12.15 ± 2.49 (96.2 ± 0.9%) |
| 29 | 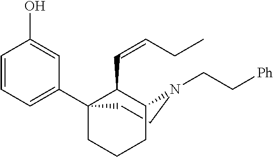 [SMILES: CC/C=C\[C@H]1[C@H]2CCC[C@]1(c1cccc(O)c1)CCN2CCc1ccccc1] | 11.59 ± 1.82 (95.8 ± 1.6%) | N/D | >10,000 | 69.04 ± 4.83 (166.6 ± 40.0%) | >10,000 | 328.28 ± 96.91 (109.5 ± 8.3%) |
| 30 | 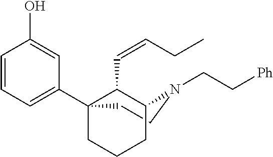 [SMILES: CC/C=C\[C@@H]1[C@H]2CCC[C@]1(c1cccc(O)c1)CCN2CCc1ccccc1] | 14.76 ± 3.57 (89.8 ± 1.9%) | N/D | >10,000 | 3223.55 ± 571.99 (182.3 ± 56.4%) | >10,000 | 282.76 ± 46.36 (103.8 ± 9.2%) |

Standards

| | | | |
|---|---|---|---|
| Morphine | 2.46 | | |
| Naltrexone | | 7.8 | Partial Agonist |

In the first set of four diastereomers in Table 2 (Compounds 19, 18, 13, and 14), three of the four diastereomers were found to be potent partial agonists with efficacies from 60-69% and the fourth was found to be a potent MOR antagonist and a weak DOR and KOR antagonist. It was about twice as potent as naltrexone. Naltrexone was noted to be a MOR antagonist and a KOR agonist. Compound 19 was identified as a target compound when used clinically being both more potent and having less undesirable KOR-mediated effects. It would not be possible to deduce or guess an activity or potency of any of the diastereomers from their structure from previous work on 5-phenylmorphans. It was entirely unexpected to find a potent antagonist, and noteworthy to find potent partial agonists with desirable efficacies.

The second set of four diastereomers, Compounds 26, 27, 22, and 23 show even more radically different and unpredicted potencies and efficacies that those in the first set. They show potencies ranging between morphine-like and 30-fold more potent than morphine (Compound 23), and one of them is a potent MOR antagonist that will need further examination in vivo. The potent MOR antagonist (Compound 26) is, as well, a moderately potent KOR antagonist.

In the third set of four enantiomers, Compound 34, was a potent MOR antagonist, a potent DOR antagonist, and a moderately potent KOR antagonist and was identified as a candidate medication for overdose from the more potent analgesics. The fourth set of diastereomers were extremely different in MOR potency. Compound 56 had the potency and efficacy that would be suitable for an improved analgesic. Additional studies would be examined for respiratory effects and self-administration.

Several potent partial MOR agonists with reasonable efficacy were identified, and three potent MOR antagonists that hold promise for improvement of current OUD treatment agents, medications, and analgesics with fewer opioid-like side-effects that have been discovered. Compound 13, a MOR high efficacy full agonist in the CAMP assay (% Eₘₐₓ=94.7) and a moderate, slightly high efficacy partial agonist in the GTP assay (% Eₘₐₓ=20.09), with high binding affinity (Kᵢ=0.5 nM) at MOR, had antinociceptive effects in vivo, in squirrel monkeys, and, at slightly lower doses, a decreased ventilatory ratio; it was found to be similar to morphine in this assay. Compound 14, a MOR partial agonist (% Eₘₐₓ=67.3) in the CAMP assay and a MOR lower efficacy partial agonist in the GTP assay (% Eₘₐₓ=10.54) had antinociceptive effects in some, but not all animals, and was without selective effects on ventilatory ratio. Compound 22, a MOR partial agonist in the CAMP assay (% Eₘₐₓ=89.5) and a partial agonist in the GTP assay (% Eₘₐₓ=17.97) had antinociceptive effects and, at slightly lower doses, had slightly lessor effects on ventilatory ratio. Both partial agonist Compounds 14 and 22 were seen to be different from morphine in those assays. They had lessor respiratory effects, in agreement with their lower intrinsic efficacy for G-protein activation. According to at least one model, partial agonists, antinociceptives with less than high efficacy, have fewer side-effects. At least two of the partial agonists cause less ventilatory effects than morphine in squirrel monkeys under “stringent” conditions, in accord with the partial agonist model.

The present disclosure includes the following aspects/embodiments/features in any order and/or in any combination:

A compound having Formula (I), its enantiomer, a racemate thereof, a salt thereof, or a prodrug thereof, or any combination thereof:

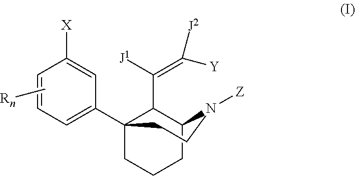

wherein in Formula (I),
X is —OR¹, —NR¹R², —CO₂R¹, —CONR¹R², —(CR¹R²)ₘ₁OH, an amide, a nitrogen-containing heteroaryl, or Ar—NHCHO; wherein
   each R¹ is H, OH, an ether, an ester, a carboxyl, a substituted or unsubstituted C₁-C₃₀ alkyl, a substituted or unsubstituted C₃-C₃₀ cycloalkyl, or a substituted or unsubstituted C₆-C₃₀ aryl;
   each R² is H, OH, an ether, an ester, a substituted or unsubstituted C₁-C₃₀ alkyl, a substituted or unsubstituted C₃-C₃₀ cycloalkyl, a substituted or unsubstituted C₂-C₃₀ alkanoyl, a substituted or unsubstituted C₄-C₃₀ cycloalkanoyl, or a substituted or unsubstituted C₆-C₃₀ aryl;
   Ar is a substituted or unsubstituted C₆-C₃₀ aryl; and
   m1 is an integer of 1 to 10;
J¹ and J² are each independently, H or a halogen, or J¹ and Y dependently constitute a bond resulting in a triple bond between their respective carbons;
Y is H, methyl, or ethyl, or J¹ and Y dependently constitute a bond resulting in a triple bond between their respective carbons;
   and
Z is H, -L-W, or —(CR³R⁴)ₘ₂W; wherein
   L is a substituted or unsubstituted C₂-C₁₀ alkenylene or a substituted or unsubstituted C₂-C₁₀ alkynylene;
   R³ and R⁴ are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, wherein any two selected from R³ and R⁴ are optionally bonded together to form a ring;
   W is H, a substituted or unsubstituted C₁-C₃₀ alkyl, a substituted or unsubstituted C₃-C₃₀ cycloalkyl, a substituted or unsubstituted spiro C₃-C₃₀ cycloalkyl. a substituted or unsubstituted C₆-C₃₀ aryl, or a substituted or unsubstituted C₁-C₃₀ heteroaryl; and
   m2 is an integer of 1 to 10;
R is H, a halogen, or a substituted or unsubstituted C₁-C₃₀ alkyl; and
n is an integer of 1 to 4.

The compound of any preceding or following embodiment/feature/aspect, its enantiomer, a racemate thereof, a prodrug thereof, or salt thereof, or any combination thereof, comprising one or more of:

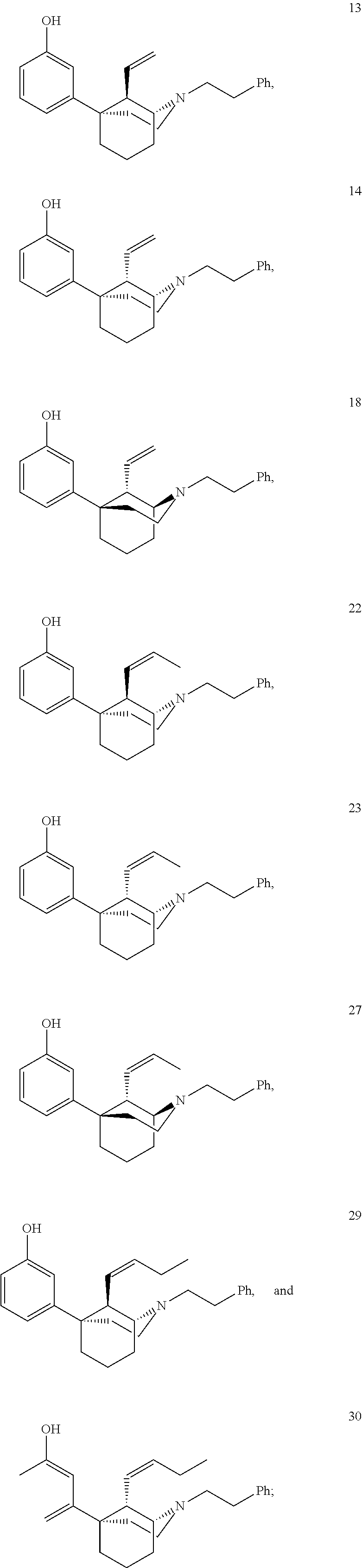

or
a salt thereof, or a prodrug thereof, or any combination thereof.

The compound of any preceding or following embodiment/feature/aspect, its enantiomer, a racemate thereof, a prodrug thereof, or salt thereof, or any combination thereof, comprising one or more of:

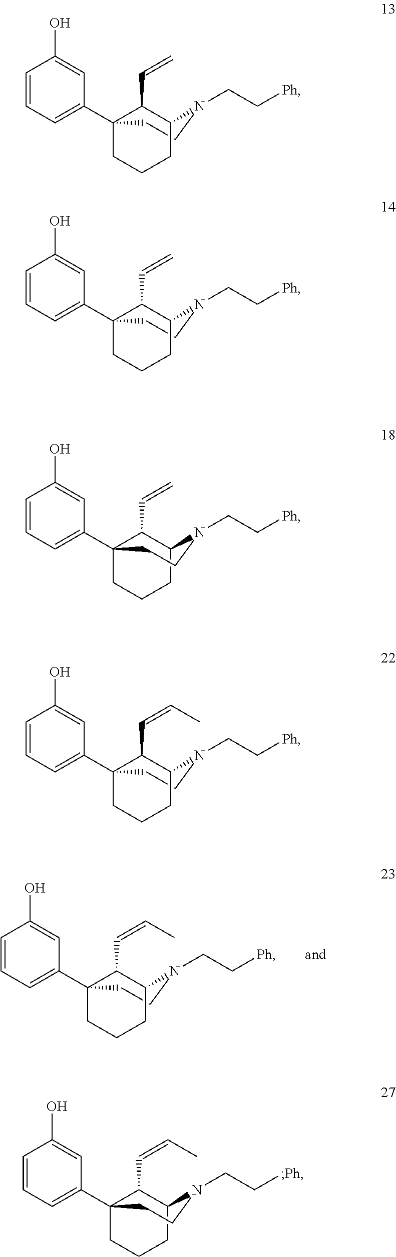

or
a salt thereof, or a prodrug thereof, or any combination thereof.

The compound of any preceding or following embodiment/feature/aspect, its enantiomer, a racemate thereof, a prodrug thereof, or salt thereof, or any combination thereof, comprising one or more of:

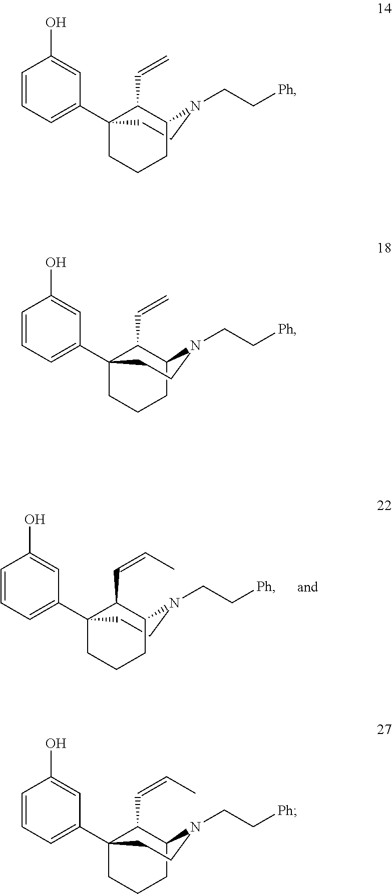 [SMILES: C=C[C@@H]1[C@H]2CCC[C@]1(c1cccc(O)c1)CCN2CCc1ccccc1 ; C=C[C@@H]1[C@@H]2CCC[C@@]1(c1cccc(O)c1)CCN2CCc1ccccc1 ; C/C=C\[C@H]1[C@H]2CCC[C@]1(c1cccc(O)c1)CCN2CCc1ccccc1 ; C/C=C\[C@@H]1[C@@H]2CCC[C@@]1(c1cccc(O)c1)CCN2CCc1ccccc1]

or
a salt thereof, or a prodrug thereof, or any combination thereof.

The compound of any preceding or following embodiment/feature/aspect, its enantiomer, a racemate thereof, a prodrug thereof, or salt thereof, or any combination thereof, comprising one or both of:

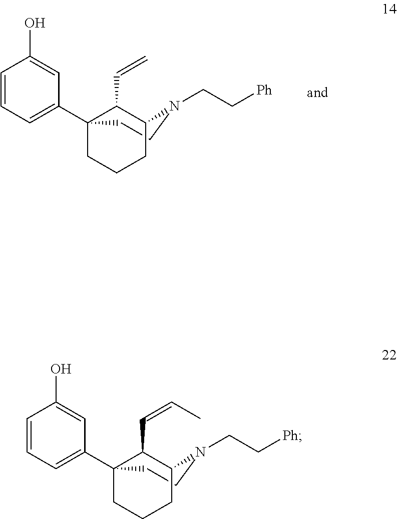 [SMILES: C=C[C@@H]1[C@H]2CCC[C@]1(c1cccc(O)c1)CCN2CCc1ccccc1 ; C/C=C\[C@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect, its enantiomer, a racemate thereof, a prodrug thereof, or salt thereof, or any combination thereof, comprising:

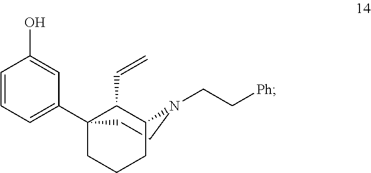 [SMILES: C=C[C@@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect, its enantiomer, a racemate thereof, a prodrug thereof, or salt thereof, or any combination thereof, comprising one or both of:

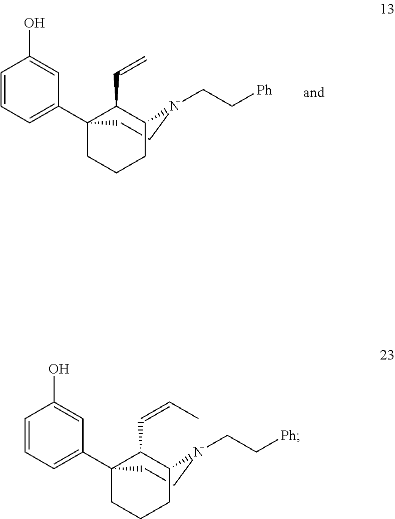 [SMILES: C=C[C@H]1[C@H]2CCC[C@]1(c1cccc(O)c1)CCN2CCc1ccccc1 ; C/C=C\[C@@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect comprising:

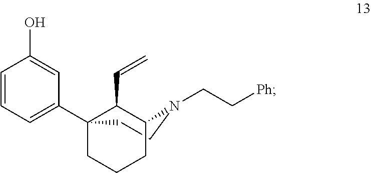 [SMILES: C=C[C@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect comprising one or more of:

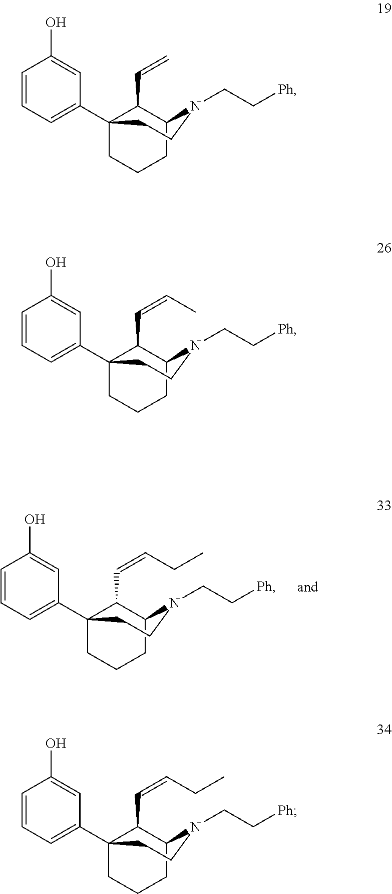 [SMILES: C=C[C@H]1[C@@H]2CCC[C@@]1(c1cccc(O)c1)CCN2CCc1ccccc1 ; C/C=C\[C@H]1[C@@H]2CCC[C@@]1(c1cccc(O)c1)CCN2CCc1ccccc1 ; CC/C=C\[C@@H]1[C@@H]2CCC[C@@]1(c1cccc(O)c1)CCN2CCc1ccccc1 ; CC/C=C\[C@H]1[C@@H]2CCC[C@@]1(c1cccc(O)c1)CCN2CCc1ccccc1]

or
a salt thereof, or a prodrug thereof, or any combination thereof.

The compound of any preceding or following embodiment/feature/aspect comprising one or more of:

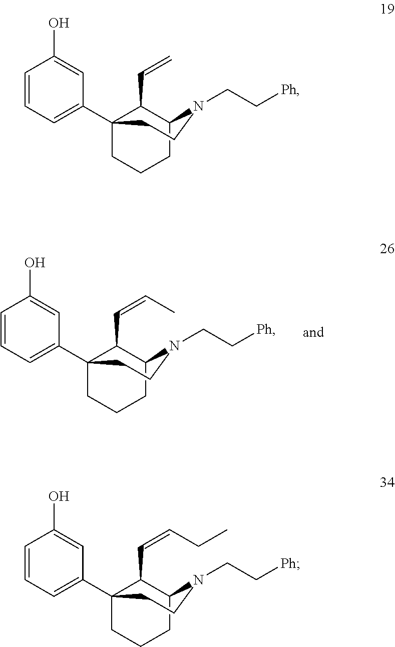 [SMILES: C=C[C@H]1[C@@H]2CCC[C@@]1(c1cccc(O)c1)CCN2CCc1ccccc1 ; C/C=C\[C@H]1[C@@H]2CCC[C@@]1(c1cccc(O)c1)CCN2CCc1ccccc1 ; CC/C=C\[C@H]1[C@@H]2CCC[C@@]1(c1cccc(O)c1)CCN2CCc1ccccc1]

or
a salt thereof, or a prodrug thereof, or any combination thereof.

The compound of any preceding or following embodiment/feature/aspect comprising one or more of:

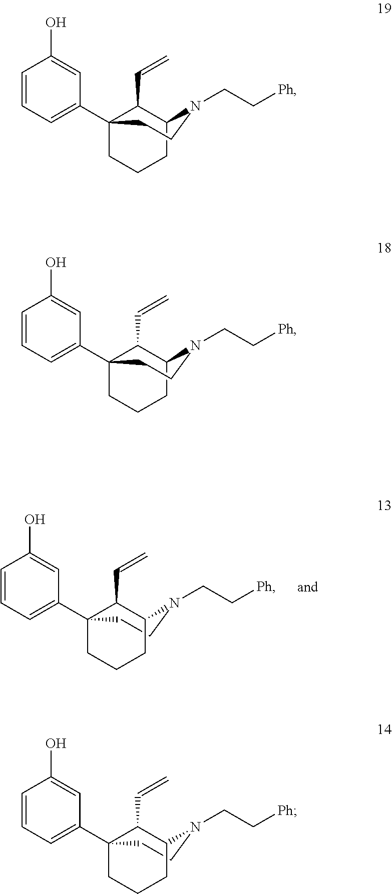

or
a salt thereof, or a prodrug thereof, or any combination thereof.

The compound of any preceding or following embodiment/feature/aspect comprising one or more of:

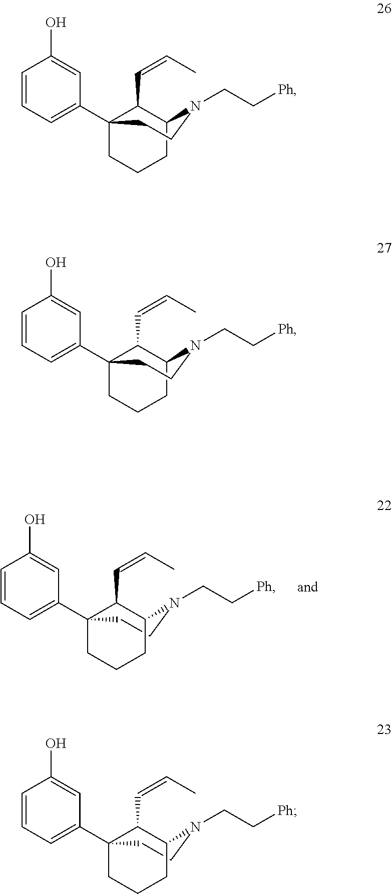 [SMILES: C/C=C\[C@H]1[C@@H]2CCC[C@@]1(c1cccc(O)c1)CCN2CCc1ccccc1 ; C/C=C\[C@@H]1[C@@H]2CCC[C@@]1(c1cccc(O)c1)CCN2CCc1ccccc1 ; C/C=C\[C@H]1[C@H]2CCC[C@]1(c1cccc(O)c1)CCN2CCc1ccccc1 ; C/C=C\[C@@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect comprising one or more of:

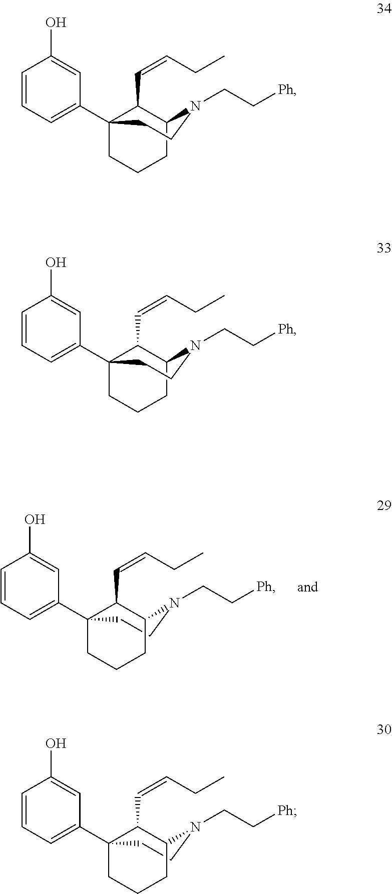

or
a salt thereof, or a prodrug thereof, or any combination thereof.

The compound of any preceding or following embodiment/feature/aspect comprising one or more of:

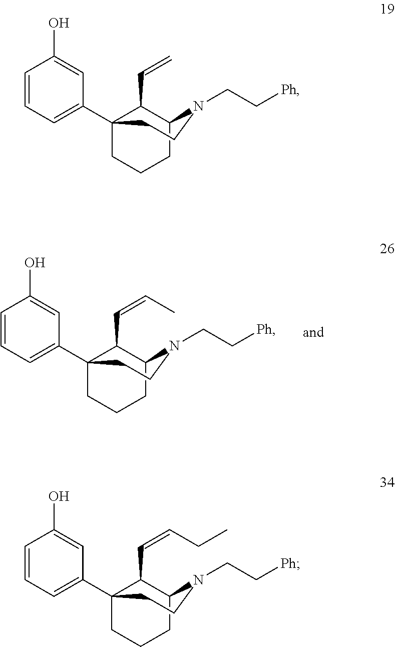 [SMILES: C=C[C@H]1[C@@H]2CCC[C@@]1(c1cccc(O)c1)CCN2CCc1ccccc1 ; C/C=C\[C@H]1[C@@H]2CCC[C@@]1(c1cccc(O)c1)CCN2CCc1ccccc1 ; CC/C=C\[C@H]1[C@@H]2CCC[C@@]1(c1cccc(O)c1)CCN2CCc1ccccc1]

or
a salt thereof, or a prodrug thereof, or any combination thereof.

The compound of any preceding or following embodiment/feature/aspect comprising one or more of:

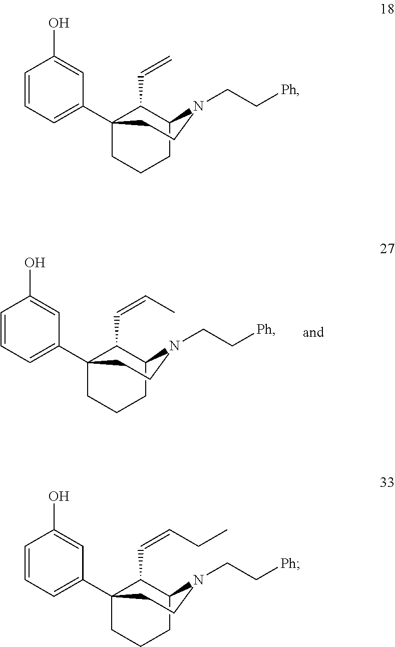 [SMILES: C=C[C@@H]1[C@@H]2CCC[C@@]1(c1cccc(O)c1)CCN2CCc1ccccc1 ; C/C=C\[C@@H]1[C@@H]2CCC[C@@]1(c1cccc(O)c1)CCN2CCc1ccccc1 ; CC/C=C\[C@@H]1[C@@H]2CCC[C@@]1(c1cccc(O)c1)CCN2CCc1ccccc1]

or
a salt thereof, or a prodrug thereof, or any combination thereof.

The compound of any preceding or following embodiment/feature/aspect comprising one or more of:

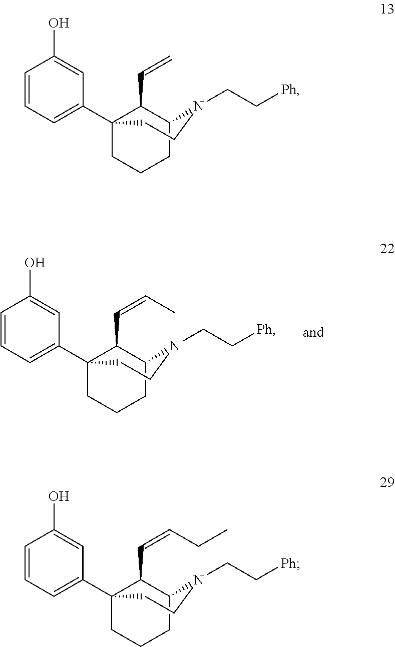 [SMILES: C=C[C@H]1[C@H]2CCC[C@]1(c1cccc(O)c1)CCN2CCc1ccccc1 ; C/C=C\[C@H]1[C@H]2CCC[C@]1(c1cccc(O)c1)CCN2CCc1ccccc1 ; CC/C=C\[C@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect comprising one or more of:

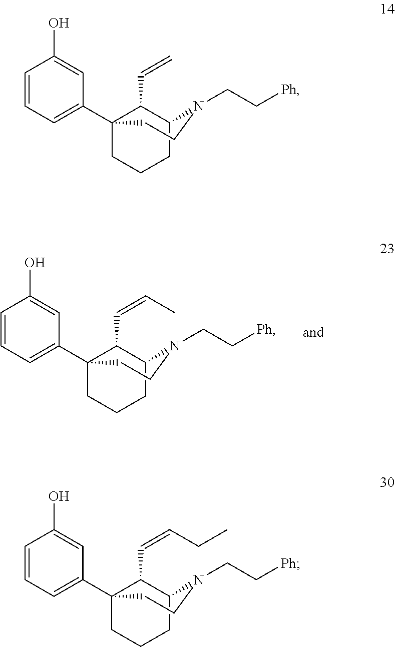 [SMILES: C=C[C@@H]1[C@H]2CCC[C@]1(c1cccc(O)c1)CCN2CCc1ccccc1 ; C/C=C\[C@@H]1[C@H]2CCC[C@]1(c1cccc(O)c1)CCN2CCc1ccccc1 ; CC/C=C\[C@@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect, its enantiomer, a racemate thereof, a salt thereof, or a prodrug thereof, or any combination thereof, wherein J¹ and J² are cis-oriented to each other and trans-oriented to Y and the piperidine.

The compound of any preceding or following embodiment/feature/aspect, its enantiomer, a racemate thereof, a salt thereof, or a prodrug thereof, or any combination thereof, wherein Formula (I) is represented by Formula (Ia):

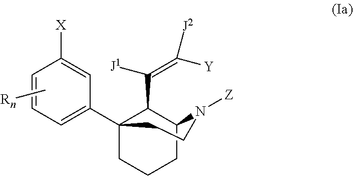

wherein in Formula (Ia), R, X, J¹, J², Y, Z, and n are the same as in of any preceding or following embodiment/feature/aspect.

The compound of any preceding or following embodiment/feature/aspect, its enantiomer, a racemate thereof, a salt thereof, or a prodrug thereof, or any combination thereof, wherein Formula (I) is represented by Formula (Ib):

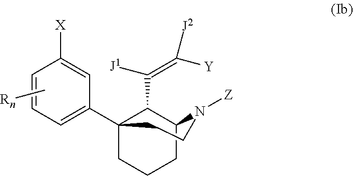

wherein in Formula (Ib), R, X, J¹, J², Y, Z, and n are the same as in of any preceding or following embodiment/feature/aspect.

The compound of any preceding or following embodiment/feature/aspect, its enantiomer, a racemate thereof, a salt thereof, or a prodrug thereof, or any combination thereof, wherein X is OH, NH₂, or NHR⁵, and R⁵ is CHO or —C(O) OR⁶, and each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Z is —(CR³R⁴)ₘ₂W, R³ and R⁴ are each H, W is a substituted or unsubstituted C₆-C₃₀ aryl, and m2 is an integer of 1 to 5.

The compound of any preceding or following embodiment/feature/aspect, its enantiomer, a racemate thereof, a salt thereof, or a prodrug thereof, or any combination thereof, wherein Z is —CH₂CH₂W, and W is unsubstituted C₆-C₃₀ aryl, C₆-C₃₀ aryl substituted with —F, —Cl, —Br, —OH, —NH₂ or —NO₂, unsubstituted C₁-C₃₀ heteroaryl, or C₁-C₃₀ heteroaryl substituted with —F, —Cl, —Br, —OH, —NH₂ or —NO₂.

The compound of any preceding or following embodiment/feature/aspect, its enantiomer, a racemate thereof, a salt thereof, or a prodrug thereof, or any combination thereof, wherein Formula (I) is represented by Formula (II):

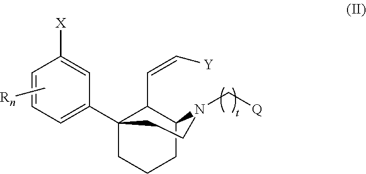

wherein in Formula (II), R, X, Y, and n are the same as in any preceding or following embodiment/feature/aspect, Q is alkyl, alkenyl, cycloalkyl, cyclopropyl, cyclopentyl, heterocyclic alkyl, bicyclic cycloalkyl, bicyclic heterocyclic alkyl, phenyl, amine, cyclic amine, piperidine, pyrrolidine, pyrrole, heterocyclic amine, bicyclic heterocyclic amine, indole, azaspirononane, spirodecane, substituted or unsubstituted C₆-C₃₀ aryl, substituted or unsubstituted C₆-C₃₀ heteroaryl, and t is 0-4 carbons.

The compound of any preceding or following embodiment/feature/aspect, its enantiomer, a racemate thereof, a salt thereof, or a prodrug thereof, or any combination thereof, wherein Formula (I) is represented by Formula (III):

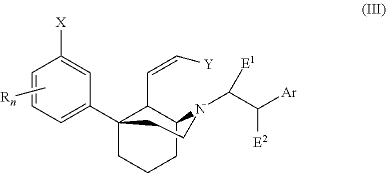

wherein in Formula (III), R, Y, and n are the same as in any preceding or following embodiment/feature/aspect, Ar is a substituted or unsubstituted C₆-C₃₀ aryl, and X is OH, NH₂, or NHR⁵, R⁵ is CHO or —C(O) OR⁶, each R⁶ is H or a substituted or unsubstituted C₁-C₃₀ alkyl, E¹ and E² are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, and any two selected from R³ and R⁴ are optionally bonded together to form a ring.

The compound of any preceding or following embodiment/feature/aspect, its enantiomer, a racemate thereof, a salt thereof, or a prodrug thereof, or any combination thereof, wherein X is an amide or a nitrogen-containing heteroaryl.

The compound of any preceding or following embodiment/feature/aspect, its enantiomer, a racemate thereof, a salt thereof, or a prodrug thereof, or any combination thereof, wherein X is imidazole or formanilide.

The compound of any preceding or following embodiment/feature/aspect, its enantiomer, a racemate thereof, a salt thereof, or a prodrug thereof, or any combination thereof, wherein R is H or halogen.

The compound of any preceding or following embodiment/feature/aspect, its enantiomer, a racemate thereof, a salt thereof, or a prodrug thereof, or any combination thereof, wherein Formula (I) is represented by Formula (IV):

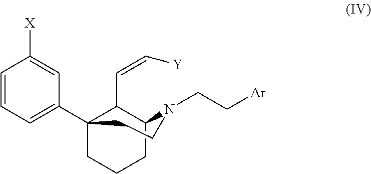

wherein in Formula (IV), Y is the same as in any preceding or following embodiment/feature/aspect, Ar is a substituted or unsubstituted C₆-C₃₀ aryl, X is OH, NH₂, or NHR⁵, R⁵ is CHO or —C(O) OR⁶, and each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Formula (I) is represented by Formula (V):

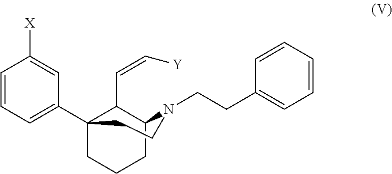

wherein in Formula (V), Y is the same as in any preceding or following embodiment/feature/aspect, and X is OH, NH₂, or NHR⁵, R⁵ is CHO or —C(O) OR⁶, and each R⁶ is Hor a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Formula (I) is represented by Formula (XI):

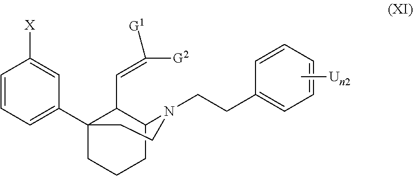

wherein in Formula (XI), X, and n are the same as in any preceding or following embodiment/feature/aspect, G¹ and G² are independently H or a halogen, X is OH, NH₂, or NHR⁵, R⁵ is CHO or —C(O) OR⁶, and each R⁶ is H or a substituted or unsubstituted C₁-C₃₀ alkyl U is a H, a halogen, or a substituted or unsubstituted C₁-C₃₀ alkyl; and n2 is an integer of 1 to 4.

The compound of any preceding or following embodiment/feature/aspect, its enantiomer, a racemate thereof, a salt thereof, or a prodrug thereof, or any combination thereof, wherein Formula (I) is represented by Formula (XII):

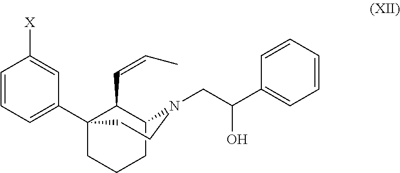

wherein in Formula (XII), X is OH, NH₂, or NHR⁵, R⁵ is CHO or —C(O) OR⁶, and each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Formula (I) is represented by Formula (XIII):

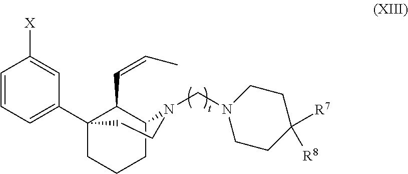

wherein in Formula (XIII), t is 0-4 carbons, X is OH, NH₂, or NHR⁵, R⁵ is CHO or —C(O) OR⁶, each R⁶ is H or a substituted or unsubstituted C₁-C₃₀ alkyl, and R⁷ and R⁸ are hydrogen, or R⁷ and R⁸ are dependently a C₁-C₆ unsubstituted heterocycloalkyl, a C₁-C₆ substituted heterocycloalkyl, an unsubstituted spiro C₃-C₃₀ heterocycloalkyl, a substituted spiro C₃-C₃₀ heterocycloalkyl, a C₁-C₆ unsubstituted heteroaryl, or a C₁-C₆ substituted heteroaryl.

The compound of any preceding or following embodiment/feature/aspect, its enantiomer, a racemate thereof, a salt thereof, or a prodrug thereof, or any combination thereof, wherein Formula (I) is represented by Formula (XIV):

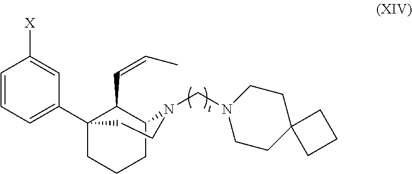

wherein in Formula (XIV), t is 0-4 carbons, and X is OH, NH₂, or NHR⁵, R⁵ is CHO or —C(O) OR⁶, and each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Formula (I) is represented by Formula (XV):

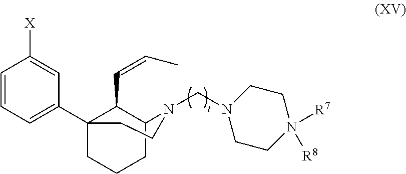

wherein in Formula (XV), t is 0-4 carbons, X is OH, NH₂, or NHR⁵, R⁵ is CHO or —C(O) OR⁶, each R⁶ is H or a substituted or unsubstituted C₁-C₃₀ alkyl, and R⁷ and R⁸ are hydrogen, or R⁷ and R⁸ are dependently a C₁-C₆ unsubstituted heterocycloalkyl, a C₁-C₆ substituted heterocycloalkyl, an unsubstituted spiro C₃-C₃₀ heterocycloalkyl, a substituted spiro C₃-C₃₀ heterocycloalkyl, a C₁-C₆ unsubstituted heteroaryl, or a C₁-C₆ substituted heteroaryl.

The compound of any preceding or following embodiment/feature/aspect, its enantiomer, a racemate thereof, a salt thereof, or a prodrug thereof, or any combination thereof, wherein Formula (I) is represented by Formula (XVI):

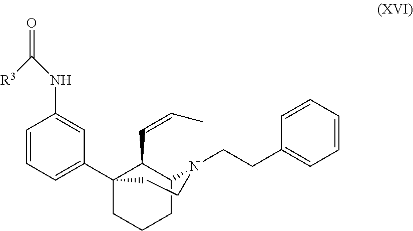

wherein in Formula (XVI), R³ is the same as in of any preceding or following embodiment/feature/aspect.

The compound of any preceding or following embodiment/feature/aspect, its enantiomer, a racemate thereof, a salt thereof, or a prodrug thereof, or any combination thereof, wherein Formula (I) is represented by Formula (XVII):

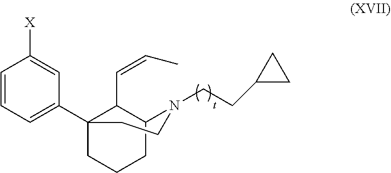

wherein in Formula (XVII), t is 0-4 carbons, and X is OH, NH₂, or NHR⁵, R⁵ is CHO or —C(O) OR⁶, and each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Formula (I) is represented by Formula (XVIII):

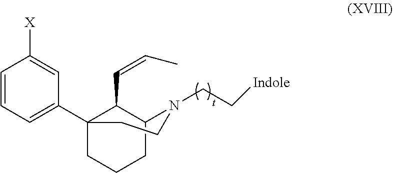

wherein in Formula (XVIII), t is 0-4 carbons, and X is OH, NH₂, or NHR⁵, R⁵ is CHO or —C(O) OR⁶, wherein each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, comprising a full agonist, a partial agonist, or an antagonist, or any combination thereof of a mu-opioid receptor.

The compound of any preceding or following embodiment/feature/aspect, its enantiomer, a racemate thereof, a salt thereof, or a prodrug thereof, or any combination thereof, lacking agonist activity against a kappa-opioid receptor, or a delta-opioid receptor, or both.

A pharmaceutical composition, comprising a therapeutically effective amount of the compound of any preceding or following embodiment/feature/aspect, its enantiomer, a racemate thereof, a salt thereof, or a prodrug thereof, or any combination thereof, together with a pharmaceutically acceptable carrier.

The pharmaceutical composition of any preceding or following embodiment/feature/aspect, wherein the pharmaceutically acceptable carrier is selected from the group consisting of binders, buffering agents, coloring agents, diluents, disintegrants, emulsifiers, flavorants, glidants, lubricants, preservatives, stabilizers, surfactants, tableting agents, and wetting agents, and combinations thereof.

The pharmaceutical composition of any preceding or following embodiment/feature/aspect, further comprising a therapeutically effective amount of an opioid.

The pharmaceutical composition of any preceding or following embodiment/feature/aspect, wherein the opioid is selected from alfentanil, buprenorphine, butorphanol, codeine, dezocine, dihydrocodeine, fentanyl, a fentanyl derivative, etonitazene, an etonitazene derivative, hydrocodone, hydromorphone, levorphanol, meperidine (pethidine), methadone, morphine, nalbuphine, oxycodone, oxymorphone, pentazocine, propiram, propoxyphene, sufentanil, tramadol, or a combination thereof.

A method of prevention or treatment of pain, comprising administering to a patient in need thereof a composition comprising a therapeutically effective amount of the compound of any preceding or following embodiment/feature/aspect, its enantiomer, a racemate thereof, a salt thereof, or a prodrug thereof, or any combination thereof optionally in combination with one or more additional active ingredients.

The method of any preceding or following embodiment/feature/aspect, wherein the one or more additional active ingredient is an opioid selected from alfentanil, buprenorphine, butorphanol, codeine, dezocine, dihydrocodeine, fentanyl, a fentanyl derivative, etonitazene, an etonitazene derivative, hydrocodone, hydromorphone, levorphanol, meperidine (pethidine), methadone, morphine, nalbuphine, oxycodone, oxymorphone, pentazocine, propiram, propoxyphene, sufentanil, tramadol, or a combination thereof.

The method of any preceding or following embodiment/feature/aspect, wherein the patient is a human.

The method of any preceding or following embodiment/feature/aspect, wherein the pain is associated with a gastrointestinal dysfunction.

The method of any preceding or following embodiment/feature/aspect, wherein the gastrointestinal dysfunction is irritable bowel syndrome, opioid-bowel dysfunction, colitis, post-operative and opioid-induced emesis, decreased gastric motility and emptying, inhibition of small and/or large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, abdominal or epigastric pain and discomfort, constipation, and delayed absorption of orally administered medications or nutritive substances.

The method of any preceding or following embodiment/feature/aspect, wherein the pain is associated with post-operative or opioid-induced ileus.

A method of treating or preventing an opioid use disorder, comprising administering to a patient in need thereof a composition comprising a therapeutically effective amount of the compound of any preceding or following embodiment/feature/aspect, its enantiomer, a salt thereof, or a prodrug thereof, a racemate thereof, or any combination thereof optionally in combination with one or more additional active ingredients.

A method of treating or preventing an opioid overdose, comprising administering to a patient in need thereof a composition comprising a therapeutically effective amount of the compound of any preceding or following embodiment/feature/aspect, its enantiomer, a racemate thereof, a salt thereof, or a prodrug thereof, or any combination thereof optionally in combination with one or more additional active ingredients.

The method of any preceding or following embodiment/feature/aspect, wherein the opioid overdose comprises respiratory depression.

The method of any preceding or following embodiment/feature/aspect, wherein the opioid overdose comprises a fentanyl overdose, a fentanyl derivative overdose, an etonitazene overdose, or an etonitazene derivative overdose, or any combination thereof.

A compound having Formula (VI), its enantiomer, a racemate thereof, a salt thereof, or a prodrug thereof, or any combination thereof:

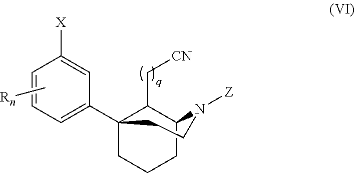

wherein in Formula (VI),
X is —OR¹, —NR¹R², —CO₂R¹, —CONR¹R², —(CR¹R²)ₘ₁OH, an amide, a nitrogen-containing heteroaryl, or Ar—NHCHO; wherein
   each R¹ is H, OH, an ether, an ester, a carboxyl, a substituted or unsubstituted C₁-C₃₀ alkyl, a substituted or unsubstituted C₃-C₃₀ cycloalkyl, or a substituted or unsubstituted C₆-C₃₀ aryl;
   each R² is H, OH, an ether, an ester, a substituted or unsubstituted C₁-C₃₀ alkyl, a substituted or unsubstituted C₃-C₃₀ cycloalkyl, a substituted or unsubstituted C₂-C₃₀ alkanoyl, a substituted or unsubstituted C₄-C₃₀ cycloalkanoyl, or a substituted or unsubstituted C₆-C₃₀ aryl;
   Ar is a substituted or unsubstituted C₆-C₃₀ aryl; and
   m1 is an integer of 1 to 10;
q is 0 to 4 carbons;
   and
Z is H, -L-W, or —(CR³R⁴)ₘ₂W; wherein
   L is a substituted or unsubstituted C₂-C₁₀ alkenylene or a substituted or unsubstituted C₂-C₁₀ alkynylene;
   R³ and R⁴ are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, wherein any two selected from R³ and R⁴ are optionally bonded together to form a ring;
   W is H, a substituted or unsubstituted C₁-C₃₀ alkyl, a substituted or unsubstituted C₃-C₃₀ cycloalkyl, a substituted or unsubstituted spiro C₃-C₃₀ cycloalkyl. a substituted or unsubstituted C₆-C₃₀ aryl, or a substituted or unsubstituted C₁-C₃₀ heteroaryl; and
   m2 is an integer of 1 to 10;
R is H, a halogen, or a substituted or unsubstituted C₁-C₃₀ alkyl; and
n is an integer of 1 to 4.

The compound of any preceding or following embodiment/feature/aspect comprising one or more of:

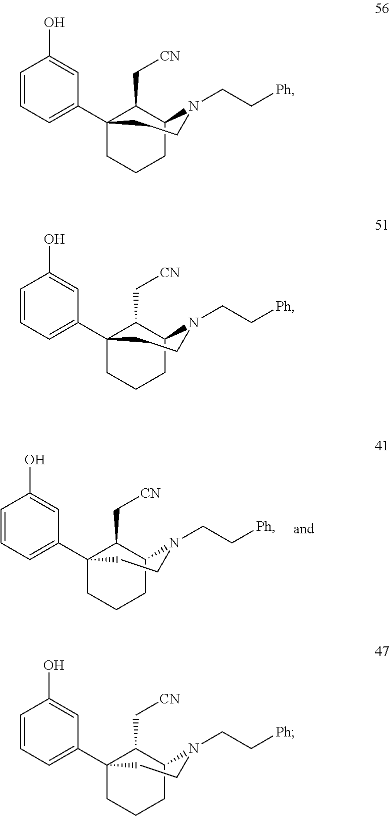 [SMILES: N#CC[C@H]1[C@@H]2CCC[C@@]1(c1cccc(O)c1)CCN2CCc1ccccc1 ; N#CC[C@@H]1[C@@H]2CCC[C@@]1(c1cccc(O)c1)CCN2CCc1ccccc1 ; N#CC[C@H]1[C@H]2CCC[C@]1(c1cccc(O)c1)CCN2CCc1ccccc1 ; N#CC[C@@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect comprising:

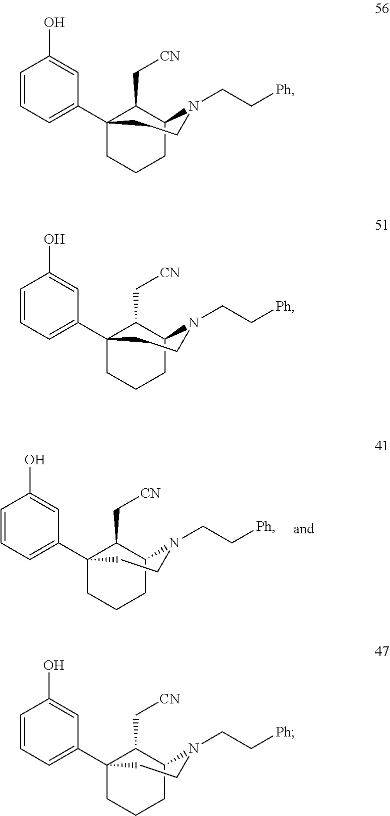 [SMILES: N#CC[C@H]1[C@@H]2CCC[C@@]1(c1cccc(O)c1)CCN2CCc1ccccc1 ; N#CC[C@@H]1[C@@H]2CCC[C@@]1(c1cccc(O)c1)CCN2CCc1ccccc1 ; N#CC[C@H]1[C@H]2CCC[C@]1(c1cccc(O)c1)CCN2CCc1ccccc1 ; N#CC[C@@H]1[C@H]2CCC[C@]1(c1cccc(O)c1)CCN2CCc1ccccc1]

or
a salt thereof, or a prodrug thereof, or any combination thereof.

The compound of any preceding or following embodiment/feature/aspect, its enantiomer, a racemate thereof, a salt thereof, or a prodrug thereof, or any combination thereof, wherein Formula (I) is represented by Formula (Via):

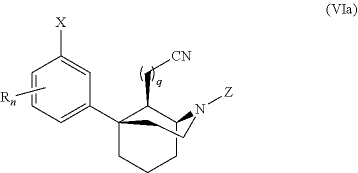

wherein in Formula (VIa), R, X, Y, Z, and n are the same as in any preceding or following embodiment/feature/aspect.

The compound of any preceding or following embodiment/feature/aspect, its enantiomer, a racemate thereof, a salt thereof, or a prodrug thereof, or any combination thereof, wherein Formula (I) is represented by Formula (VIb):

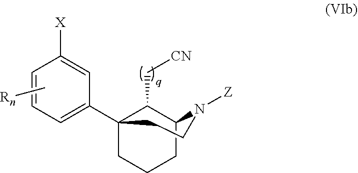

wherein in Formula (VIb), R, X, Z, and n are the same as in any preceding or following embodiment/feature/aspect.

The compound of any preceding or following embodiment/feature/aspect, its enantiomer, a racemate thereof, a salt thereof, or a prodrug thereof, or any combination thereof, wherein X is OH, NH₂, or NHR⁵, R⁵ is CHO or —C(O) OR⁶, and each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Z is —(CR⁷R⁸)ₘ₂W, R³ and R⁴ are each H, W is a substituted or unsubstituted C₆-C₃₀ aryl, and m2 is an integer of 1 to 5.

The compound of any preceding or following embodiment/feature/aspect, its enantiomer, a racemate thereof, a salt thereof, or a prodrug thereof, or any combination thereof, wherein Z is —CH₂CH₂W, and W is unsubstituted C₆-C₃₀ aryl, C₆-C₃₀ aryl substituted with —F, —Cl, —Br, —OH, —NH₂ or —NO₂, unsubstituted C₁-C₃₀ heteroaryl, or C₁-C₃₀ heteroaryl substituted with —F, —Cl, —Br, —OH, —NH₂ or —NO₂.

The compound of any preceding or following embodiment/feature/aspect, its enantiomer, a racemate thereof, a salt thereof, or a prodrug thereof, or any combination thereof, wherein Formula (I) is represented by Formula (VII):

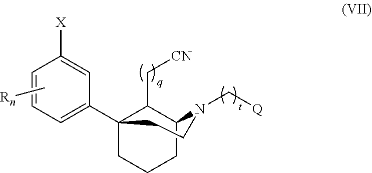

wherein in Formula (VII), R, X, q, and n are the same as in any preceding or following embodiment/feature/aspect, Q is alkyl, alkenyl, cycloalkyl, cyclopropyl, cyclopentyl, heterocyclic alkyl, bicyclic cycloalkyl, bicyclic heterocyclic alkyl, phenyl, amine, cyclic amine, piperidine, pyrrolidine, pyrrole, heterocyclic amine, bicyclic heterocyclic amine, indole, azaspirononane, spirodecane, substituted or unsubstituted C₆-C₃₀ aryl, substituted or unsubstituted C₆-C₃₀ heteroaryl, and t is 0-4 carbons.

The compound of any preceding or following embodiment/feature/aspect, its enantiomer, a racemate thereof, a salt thereof, or a prodrug thereof, or any combination thereof, wherein Formula (I) is represented by Formula (VIII):

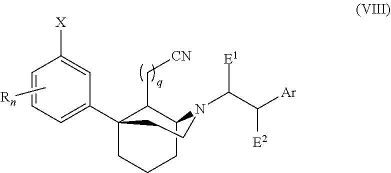

wherein in Formula (VIII), R, q, and n are the same as in any preceding or following embodiment/feature/aspect, Ar is a substituted or unsubstituted C₆-C₃₀ aryl, and X is OH, NH₂, or NHR⁵, R⁵ is CHO or —C(O) OR⁶, wherein each R⁶ is H or a substituted or unsubstituted C₁-C₃₀ alkyl, and E¹ and E² are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, and any two selected from R³ and R⁴ are optionally bonded together to form a ring.

The compound of any preceding or following embodiment/feature/aspect, its enantiomer, a racemate thereof, a salt thereof, or a prodrug thereof, or any combination thereof, wherein X is an amide or a nitrogen-containing heteroaryl.

The compound of any preceding or following embodiment/feature/aspect, its enantiomer, a racemate thereof, a salt thereof, or a prodrug thereof, or any combination thereof, wherein X is imidazole or formanilide.

The compound of any preceding or following embodiment/feature/aspect, its enantiomer, a racemate thereof, a salt thereof, or a prodrug thereof, or any combination thereof, wherein R is H or halogen.

The compound of any preceding or following embodiment/feature/aspect, its enantiomer, a racemate thereof, a salt thereof, or a prodrug thereof, or any combination thereof, wherein Formula (I) is represented by Formula (IX):

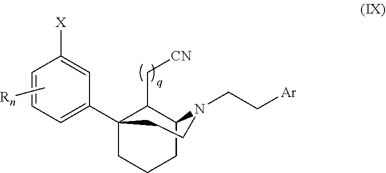

wherein in Formula (IX), q is the same as in any preceding or following embodiment/feature/aspect, Ar is a substituted or unsubstituted C₆-C₃₀ aryl, and X is OH, NH₂, or NHR⁵, R⁵ is CHO or —C(O) OR⁶, and each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Formula (I) is represented by Formula (X):

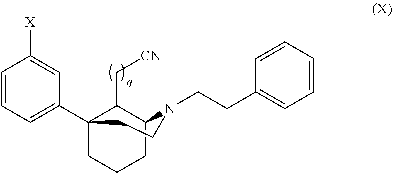

wherein in Formula (X), q is the same as in any preceding or following embodiment/feature/aspect, and X is OH, NH₂, or NHR⁵, R⁵ is CHO or —C(O) OR⁶, and each R⁶ is H or a substituted or unsubstituted C₁-C₃₀ alkyl.

The compound of any preceding or following embodiment/feature/aspect, its enantiomer, a racemate thereof, a salt thereof, or a prodrug thereof, or any combination thereof, wherein Formula (VI) is represented by Formula (XIX):

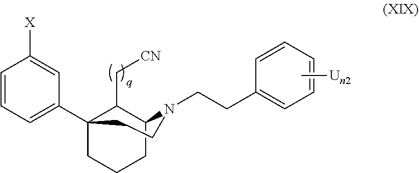

wherein in Formula (XIX), q is the same as in any preceding or following embodiment/feature/aspect, and U is a H, a halogen, or a substituted or unsubstituted C₁-C₃₀ alkyl, and n2 is an integer of 1 to 4.

The compound of any preceding or following embodiment/feature/aspect, its enantiomer, a racemate thereof, a salt thereof, or a prodrug thereof, or any combination thereof, comprising a full agonist, a partial agonist, or an antagonist, or any combination thereof of a mu-opioid receptor.

The compound of any preceding or following embodiment/feature/aspect, its enantiomer, a racemate thereof, a salt thereof, or a prodrug thereof, or any combination thereof, lacking agonist activity against a kappa-opioid receptor, or a delta-opioid receptor, or both.

A pharmaceutical composition, comprising a therapeutically effective amount of the compound of any preceding or following embodiment/feature/aspect, its enantiomer, a racemate thereof, a salt thereof, or a prodrug thereof, or any combination thereof, together with a pharmaceutically acceptable carrier.

The pharmaceutical composition of any preceding or following embodiment/feature/aspect, wherein the pharmaceutically acceptable carrier is selected from the group consisting of binders, buffering agents, coloring agents, diluents, disintegrants, emulsifiers, flavorants, glidants, lubricants, preservatives, stabilizers, surfactants, tableting agents, and wetting agents, and combinations thereof.

The pharmaceutical composition of any preceding or following embodiment/feature/aspect, further comprising a therapeutically effective amount of an opioid.

The pharmaceutical composition of any preceding or following embodiment/feature/aspect, wherein the opioid is selected from alfentanil, buprenorphine, butorphanol, codeine, dezocine, dihydrocodeine, fentanyl, a fentanyl derivative, etonitazene, an etonitazene derivative, hydrocodone, hydromorphone, levorphanol, meperidine (pethidine), methadone, morphine, nalbuphine, oxycodone, oxymorphone, pentazocine, propiram, propoxyphene, sufentanil, tramadol, or a combination thereof.

A method of prevention or treatment of pain, comprising administering to the patient a composition comprising a therapeutically effective amount of any preceding or following embodiment/feature/aspect, its enantiomer, a racemate thereof, a salt thereof, or a prodrug thereof, or any combination thereof, optionally in combination with one or more additional active ingredients.

The method of any preceding or following embodiment/feature/aspect, wherein the one or more additional active ingredient is an opioid selected from alfentanil, buprenorphine, butorphanol, codeine, dezocine, dihydrocodeine, fentanyl, a fentanyl derivative, etonitazene, an etonitazene derivative, hydrocodone, hydromorphone, levorphanol, meperidine (pethidine), methadone, morphine, nalbuphine, oxycodone, oxymorphone, pentazocine, propiram, propoxyphene, sufentanil, tramadol, or a combination thereof.

The method of any preceding or following embodiment/feature/aspect, wherein the patient is a human.

The method of any preceding or following embodiment/feature/aspect, wherein the pain is associated with a gastrointestinal dysfunction.

The method of any preceding or following embodiment/feature/aspect, wherein the gastrointestinal dysfunction is irritable bowel syndrome, opioid-bowel dysfunction, colitis, post-operative and opioid-induced emesis, decreased gastric motility and emptying, inhibition of small and/or large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, abdominal or epigastric pain and discomfort, constipation, and delayed absorption of orally administered medications or nutritive substances.

The method of any preceding or following embodiment/feature/aspect, wherein the pain is associated with post-operative or opioid-induced ileus.

A method of treating or preventing an opioid use disorder, comprising administering to a patient in need thereof a composition comprising a therapeutically effective amount of the compound of any preceding or following embodiment/feature/aspect, its enantiomer, a racemate thereof, or any combination thereof optionally in combination with one or more additional active ingredients.

A method of treating or preventing an opioid overdose, comprising administering to a patient in need thereof a composition comprising a therapeutically effective amount of the compound of any preceding amend, its enantiomer, a racemate thereof, a salt thereof, or a prodrug thereof, or any combination thereof optionally in combination with one or more additional active ingredients.

The method of any preceding or following embodiment/feature/aspect, wherein the opioid overdose comprises respiratory depression.

The method of any preceding or following embodiment/feature/aspect, wherein the opioid overdose comprises a fentanyl overdose, a fentanyl derivative overdose, an etonitazene overdose, or an etonitazene derivative overdose, or any combination thereof.

The present disclosure can include any combination of these various features or embodiments above and/or below as set forth in sentences and/or paragraphs. Any combination of disclosed features herein is considered part of the present disclosure. Further, when an amount, concentration, or other value or parameter is given as either a range, or a list of upper values and lower values, all ranges formed from any pair of any upper range limit or value and any lower range limit or value are also disclosed, regardless of whether ranges are separately disclosed. Where a range of numerical values is recited herein, unless otherwise stated, the range is intended to include the endpoints thereof, and all subranges, integers, and fractions within the range. The scope of the disclosure is not limited to the specific values recited in a range. All references cited in this specification are herein incorporated by reference as though each reference was specifically and individually indicated to be incorporated by reference. Each of the elements described herein, or two or more together, are also within the scope of the present disclosure.

1. A compound having Formula (I), its enantiomer, a racemate thereof, a salt thereof, or a prodrug thereof, or any combination thereof: 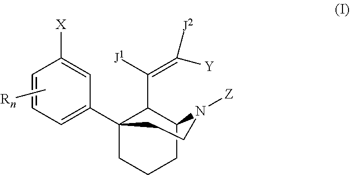
wherein in Formula (I),
X is —OR¹, —NR¹R², —CO₂R¹, —CONR¹R², —(CR¹R²)ₘ₁OH, an amide, a nitrogen-containing heteroaryl, or Ar—NHCHO; wherein
each R¹ is H, OH, an ether, an ester, a carboxyl, a substituted or unsubstituted C₁-C₃₀ alkyl, a substituted or unsubstituted C₃-C₃₀ cycloalkyl, or a substituted or unsubstituted C₆-C₃₀ aryl;
each R² is H, OH, an ether, an ester, a substituted or unsubstituted C₁-C₃₀ alkyl, a substituted or unsubstituted C₃-C₃₀ cycloalkyl, a substituted or unsubstituted C₂-C₃₀ alkanoyl, a substituted or unsubstituted C₄-C₃₀ cycloalkanoyl, or a substituted or unsubstituted C₆-C₃₀ aryl;
Ar is a substituted or unsubstituted C₆-C₃₀ aryl; and
m1 is an integer of 1 to 10;
J¹ and J² are each independently, H or a halogen, or J¹ and Y dependently constitute a bond resulting in a triple bond between their respective carbons;
Y is H, methyl, or ethyl, or J¹ and Y dependently constitute a bond resulting in a triple bond between their respective carbons;
and
Z is H, -L-W, or —(CR³R⁴)ₘ₂W; wherein
L is a substituted or unsubstituted C₂-C₁₀ alkenylene or a substituted or unsubstituted C₂-C₁₀ alkynylene;
R³ and R⁴ are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, wherein any two selected from R³ and R⁴ are optionally bonded together to form a ring;
W is H, a substituted or unsubstituted C₁-C₃₀ alkyl, a substituted or unsubstituted C₃-C₃₀ cycloalkyl, a substituted or unsubstituted spiro C₃-C₃₀ cycloalkyl. a substituted or unsubstituted C₆-C₃₀ aryl, or a substituted or unsubstituted C₁-C₃₀ heteroaryl; and
m2 is an integer of 1 to 10;
R is H, a halogen, or a substituted or unsubstituted C₁-C₃₀ alkyl; and
n is an integer of 1 to 4.

2. The compound of claim 1 comprising one or more of: 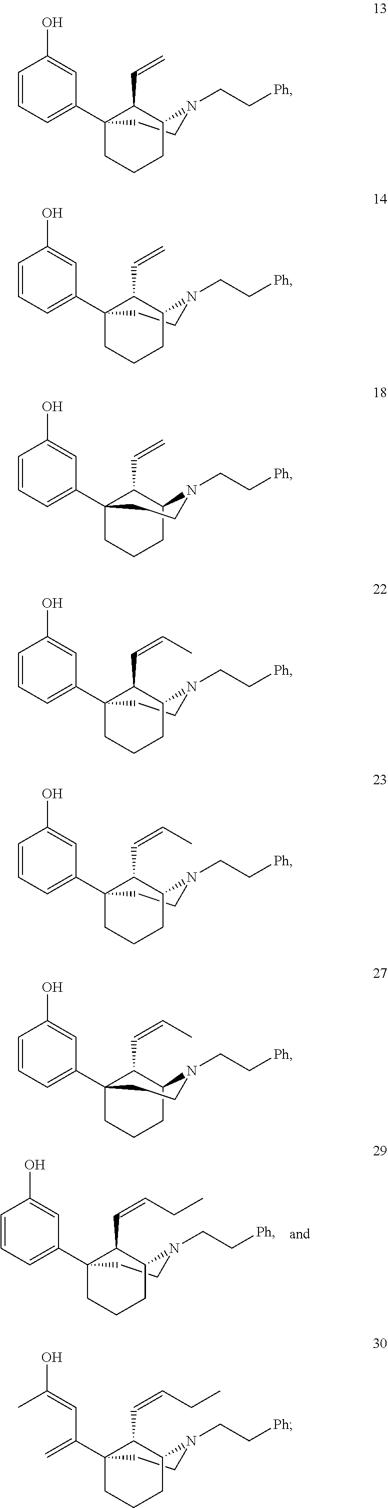
or
a salt thereof, or a prodrug thereof, or any combination thereof.

3.-18. (canceled)

19. The compound of claim 1, its enantiomer, a racemate thereof, a salt thereof, or a prodrug thereof, or any combination thereof, wherein Formula (I) is represented by Formula (Ia): 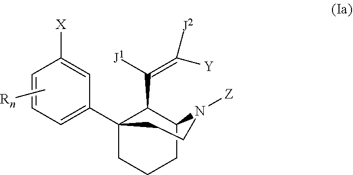
wherein Formula (la), R, X, J¹, J², Y, Z, and n are the same as in claim 1.

20. The compound of claim 1, its enantiomer, a racemate thereof, a salt thereof, or a prodrug thereof, or any combination thereof, wherein Formula (I) is represented by Formula (Ib): 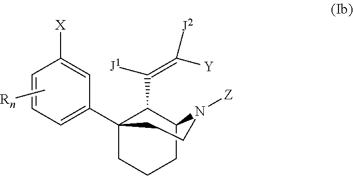
wherein Formula (Ib), R, X, J¹, J², Y, Z, and n are the same as in claim 1.

21.-23. (canceled)

24. The compound of claim 1, its enantiomer, a racemate thereof, a salt thereof, or a prodrug thereof, or any combination thereof, wherein Formula (I) is represented by Formula (II): 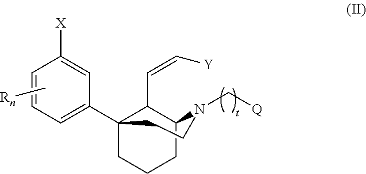
wherein Formula (II), R, X, Y, and n are the same as in claim 1, Q is alkyl, alkenyl, cycloalkyl, cyclopropyl, cyclopentyl, heterocyclic alkyl, bicyclic cycloalkyl, bicyclic heterocyclic alkyl, phenyl, amine, cyclic amine, piperidine, pyrrolidine, pyrrole, heterocyclic amine, bicyclic heterocyclic amine, indole, azaspirononane, spirodecane, substituted or unsubstituted C₆-C₃₀ aryl, substituted or unsubstituted C₆-C₃₀ heteroaryl, and t is 0-4 carbons.

25. The compound of claim 1, its enantiomer, a racemate thereof, a salt thereof, or a prodrug thereof, or any combination thereof, wherein Formula (I) is represented by Formula (III): 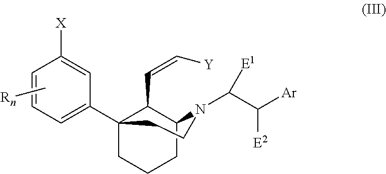
wherein Formula (III), R, Y, and n are the same as in claim 1, Ar is a substituted or unsubstituted C₆-C₃₀ aryl, and X is OH, NH₂, or NHR⁵, R⁵ is CHO or —C(O) OR⁶, each R⁶ is H or a substituted or unsubstituted C₁-C₃₀ alkyl, E¹ and E² are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, and any two selected from R³ and R⁴ are optionally bonded together to form a ring.

26.-28. (canceled)

29. The compound of claim 1, its enantiomer, a racemate thereof, a salt thereof, or a prodrug thereof, or any combination thereof, wherein Formula (I) is represented by Formula (IV): 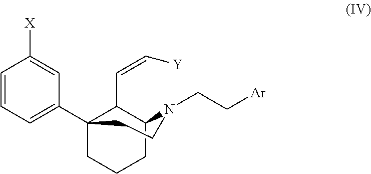
wherein Formula (IV), Y is the same as in claim 1, Ar is a substituted or unsubstituted C₆-C₃₀ aryl, X is OH, NH₂, or NHR⁵, R⁵ is CHO or —C(O) OR⁶, and each R⁶ is H or a substituted or unsubstituted C₁-C₃₀ alkyl.

30. The compound of claim 1, its enantiomer, a racemate thereof, a salt thereof, or a prodrug thereof, or any combination thereof, wherein Formula (I) is represented by Formula (V): 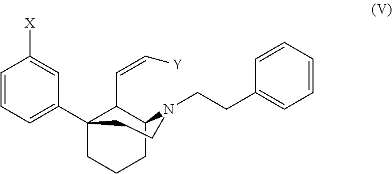
wherein Formula (V), Y is the same as in claim 1, and X is OH, NH₂, or NHR⁵, R⁵ is CHO or —C(O) OR⁶, and each R⁶ is H or a substituted or unsubstituted C₁-C₃₀ alkyl.

31. The compound of claim 1, its enantiomer, a racemate thereof, a salt thereof, or a prodrug thereof, or any combination thereof, wherein Formula (I) is represented by Formula (XI): 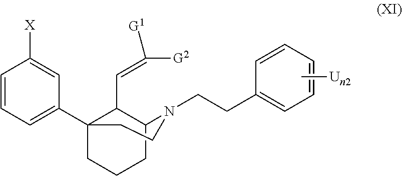
wherein Formula (XI), X, and n are the same as in claim 1, G¹ and G² are independently H or a halogen, X is OH, NH₂, or NHR⁵, R⁵ is CHO or —C(O) OR⁶, and each R⁶ is H or a substituted or unsubstituted C₁-C₃₀ alkyl U is a H, a halogen, or a substituted or unsubstituted C₁-C₃₀ alkyl; and n2 is an integer of 1 to 4.

32. The compound of claim 1, its enantiomer, a racemate thereof, a salt thereof, or a prodrug thereof, or any combination thereof, wherein Formula (I) is represented by Formula (XII): 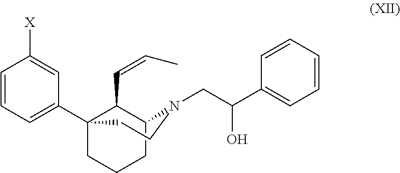
wherein in Formula (XII), X is OH, NH₂, or NHR⁵, R⁵ is CHO or —C(O) OR⁶, and each R⁶ is H or a substituted or unsubstituted C₁-C₃₀ alkyl.

33. The compound of claim 1, its enantiomer, a racemate thereof, a salt thereof, or a prodrug thereof, or any combination thereof, wherein Formula (I) is represented by Formula (XIII): 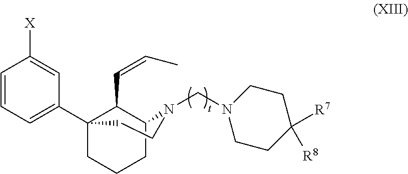
wherein in Formula (XIII), t is 0-4 carbons, X is OH, NH₂, or NHR⁵, R⁵ is CHO or —C(O) OR⁶, each R⁶ is H or a substituted or unsubstituted C₁-C₃₀ alkyl, and R⁷ and R⁸ are hydrogen, or R⁷ and R⁸ are dependently a C₁-C₆ unsubstituted heterocycloalkyl, a C₁-C₆ substituted heterocycloalkyl, an unsubstituted spiro C₃-C₃₀ heterocycloalkyl, a substituted spiro C₃-C₃₀ heterocycloalkyl, a C₁-C₆ unsubstituted heteroaryl, or a C₁-C₆ substituted heteroaryl.

34. The compound of claim 1, its enantiomer, a racemate thereof, a salt thereof, or a prodrug thereof, or any combination thereof, wherein Formula (I) is represented by Formula (XIV): 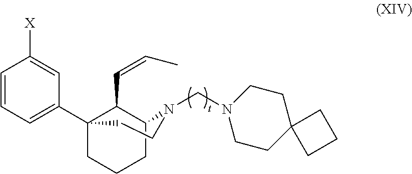
wherein in Formula (XIV), t is 0-4 carbons, and X is OH, NH₂, or NHR⁵, R⁵ is CHO or —C(O) OR⁶, and each R⁶ is H or a substituted or unsubstituted C₁-C₃₀ alkyl.

35. The compound of claim 1, its enantiomer, a racemate thereof, a salt thereof, or a prodrug thereof, or any combination thereof, wherein Formula (I) is represented by Formula (XV): 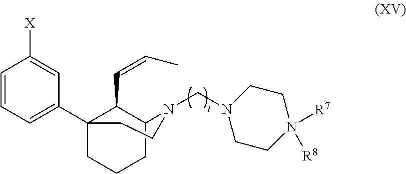
wherein in Formula (XV), t is 0-4 carbons, X is OH, NH₂, or NHR⁵, R⁵ is CHO or —C(O) OR⁶, each R⁶ is H or a substituted or unsubstituted C₁-C₃₀ alkyl, and R⁷ and R⁸ are hydrogen, or R⁷ and R⁸ are dependently a C₁-C₆ unsubstituted heterocycloalkyl, a C₁-C₆ substituted heterocycloalkyl, an unsubstituted spiro C₃-C₃₀ heterocycloalkyl, a substituted spiro C₃-C₃₀ heterocycloalkyl, a C₁-C₆ unsubstituted heteroaryl, or a C₁-C₆ substituted heteroaryl.

36. The compound of claim 1, its enantiomer, a racemate thereof, a salt thereof, or a prodrug thereof, or any combination thereof, wherein Formula (I) is represented by Formula (XVI): 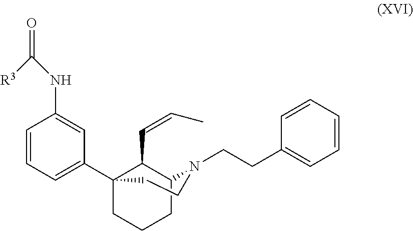
wherein Formula (XVI), R³ is the same as in claim 1.

37. The compound of claim 1, its enantiomer, a racemate thereof, a salt thereof, or a prodrug thereof, or any combination thereof, wherein Formula (I) is represented by Formula (XVII): 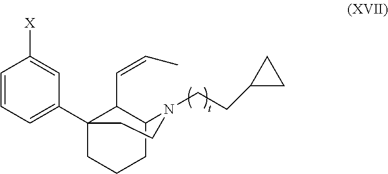
wherein in Formula (XVII), t is 0-4 carbons, and X is OH, NH₂, or NHR⁵, R⁵ is CHO or —C(O) OR⁶, and each R⁶ is H or a substituted or unsubstituted C₁-C₃₀ alkyl.

38. The compound of claim 1, its enantiomer, a racemate thereof, a salt thereof, or a prodrug thereof, or any combination thereof, wherein Formula (I) is represented by Formula (XVIII): 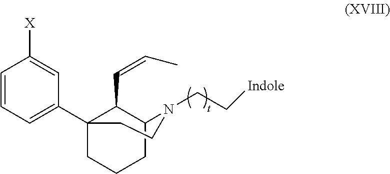
wherein in Formula (XVIII), t is 0-4 carbons, and X is OH, NH₂, or NHR⁵, R⁵ is CHO or —C(O) OR⁶, wherein each R⁶ is H or a substituted or unsubstituted C₁-C₃₀ alkyl.

39.-40. (canceled)

41. A pharmaceutical composition, comprising a therapeutically effective amount of the compound of claim 1, its enantiomer, a racemate thereof, a salt thereof, or a prodrug thereof, or any combination thereof, together with a pharmaceutically acceptable carrier.

42.-44. (canceled)

45. A method of prevention or treatment of pain, comprising administering to a patient in need thereof a composition comprising a therapeutically effective amount of the compound of claim 1, its enantiomer, a racemate thereof, a salt thereof, or a prodrug thereof, or any combination thereof optionally in combination with one or more additional active ingredients.

46.-54. (canceled)

55. A compound having Formula (VI), its enantiomer, a racemate thereof, a salt thereof, or a prodrug thereof, or any combination thereof: 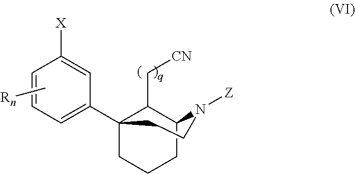
wherein in Formula (VI),
X is —OR¹, —NR¹R², —CO₂R¹, —CONR¹R², —(CR¹R²)ₘ₁OH, an amide, a nitrogen-containing heteroaryl, or Ar—NHCHO; wherein
each R¹ is H, OH, an ether, an ester, a carboxyl, a substituted or unsubstituted C₁-C₃₀ alkyl, a substituted or unsubstituted C₃-C₃₀ cycloalkyl, or a substituted or unsubstituted C₆-C₃₀ aryl;
each R² is H, OH, an ether, an ester, a substituted or unsubstituted C₁-C₃₀ alkyl, a substituted or unsubstituted C₃-C₃₀ cycloalkyl, a substituted or unsubstituted C₂-C₃₀ alkanoyl, a substituted or unsubstituted C₄-C₃₀ cycloalkanoyl, or a substituted or unsubstituted C₆-C₃₀ aryl;
Ar is a substituted or unsubstituted C₆-C₃₀ aryl; and
m1 is an integer of 1 to 10;
q is 0 to 4 carbons;
and
Z is H, -L-W, or —(CR³R⁴)ₘ₂W; wherein
L is a substituted or unsubstituted C₂-C₁₀ alkenylene or a substituted or unsubstituted C₂-C₁₀ alkynylene;
R³ and R⁴ are each independently H, OH, O (ketone), a halogen, an amine, a substituted or unsubstituted C₁-C₃₀ alkyl, a substituted or unsubstituted C₃-C₃₀ cycloalkyl, a substituted or unsubstituted C₆-C₃₀ aryl, or a substituted or unsubstituted C₁-C₃₀ heteroaryl, wherein any two selected from R³ and R⁴ are optionally bonded together to form a ring;
W is H, a substituted or unsubstituted C₁-C₃₀ alkyl, a substituted or unsubstituted C₃-C₃₀ cycloalkyl, a substituted or unsubstituted spiro C₃-C₃₀ cycloalkyl. a substituted or unsubstituted C₆-C₃₀ aryl, or a substituted or unsubstituted C₁-C₃₀ heteroaryl; and
m2 is an integer of 1 to 10;
R is H, a halogen, or a substituted or unsubstituted C₁-C₃₀ alkyl; and
n is an integer of 1 to 4.

56. The compound of claim 55 comprising one or more of: 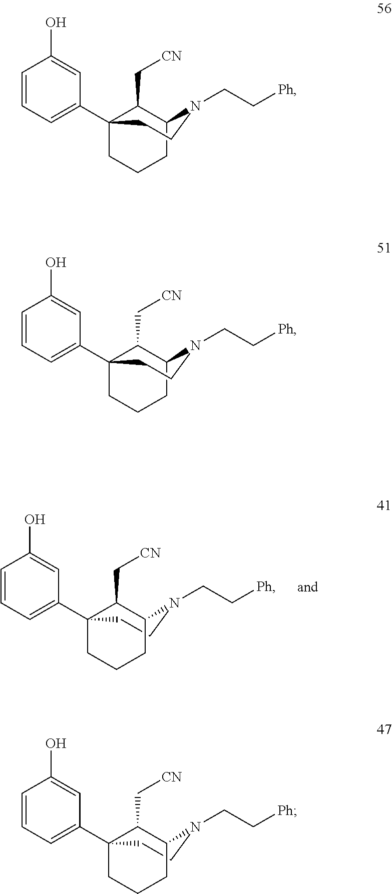 [SMILES: N#CC[C@H]1[C@@H]2CCC[C@@]1(c1cccc(O)c1)CCN2CCc1ccccc1 ; N#CC[C@@H]1[C@@H]2CCC[C@@]1(c1cccc(O)c1)CCN2CCc1ccccc1 ; N#CC[C@H]1[C@H]2CCC[C@]1(c1cccc(O)c1)CCN2CCc1ccccc1 ; N#CC[C@@H]1[C@H]2CCC[C@]1(c1cccc(O)c1)CCN2CCc1ccccc1]
or
a salt thereof, or a prodrug thereof, or any combination thereof.

57.-86. (canceled)
